SEQUENCE LISTING (2 sequences)

SEQ ID NO: 1 - protein, 15 aa, human
MKKHHHHHHLVPRGS

SEQ ID NO: 2 - protein, 25 aa, Artificial Sequence
  Biotinylated peptide
CPSSHSSLTERHKILHRLLQEGSPS

(19) United States
(12) Patent Application Publication
(10) Pub. No.: US 2014/0155419 A1
(43) Pub. Date: Jun. 5, 2014

(54) COMPOUNDS AND METHODS

(71) Applicants: Erkan Baloglu, Stoneham, MA (US); Shomir Ghosh, Cambridge, MA (US); Mercedes Lobera, Cambridge, MA (US); Darby R. Schmidt, Arlington, MA (US)

(72) Inventors: Erkan Baloglu, Stoneham, MA (US); Shomir Ghosh, Cambridge, MA (US); Mercedes Lobera, Cambridge, MA (US); Darby R. Schmidt, Arlington, MA (US)

(21) Appl. No.: 14/235,481

(22) PCT Filed: Jul. 27, 2012
(86) PCT No.: PCT/US2012/048553

§ 371 Date: Jan. 28, 2014

Related U.S. Application Data
(60) Provisional application No. 61/533,897, filed on Sep. 13, 2011.
(60) Provisional application No. 61/521,812, filed on Aug. 10, 2011.
(60) Provisional application No. 61/513,179, filed on Jul. 29, 2011.

(51) Int. Cl. C07D 239/52; C07D 213/40; C07D 239/26; C07D 213/30

(52) U.S. Cl. CPC C07D 239/52; C07D 213/30; C07D 213/40; C07D 239/26
USPC 514/269; 546/337; 514/357; 546/265; 514/332; 544/335; 514/256; 544/319

(57) ABSTRACT

The present invention relates to novel retinoid-related orphan receptor gamma (RORγ) modulators and their use in the treatment of diseases mediated by RORγ.

The present invention relates to novel retinoid-related orphan receptor gamma (RORγ) modulators and their use in the treatment of diseases mediated by RORγ.

### BACKGROUND OF THE INVENTION

Retinoid-related orphan receptors (RORs) are transcription factors which belong to the steroid hormone nuclear receptor superfamily (Jetten & Joo (2006) Adv. Dev. Biol. 16:313-355). The ROR family consists of three members, ROR alpha (RORα), ROR beta (RORβ), and ROR gamma (RORγ), each encoded by a separate gene (RORA, RORB, and RORC, respectively). RORs contain four principal domains shared by the majority of nuclear receptors: an N-terminal A/B domain, a DNA-binding domain, a hinge domain, and a ligand binding domain. Each ROR gene generates several isoforms which differ only in their N-terminal A/B domain. Two isoforms of RORγ have been identified: RORγ1 and RORγt (also known as RORγ2). RORγ is a term used to describe both RORγ1 and/or RORγt.

While RORγ1 is expressed in a variety of tissues including thymus, muscle, kidney and liver, RORγt is exclusively expressed in the cells of the immune system. RORγt has been identified as a key regulator of Th17 cell differentiation. Th17 cells are a subset of T helper cells which produce IL-17 and other proinflammatory cytokines. Th17 cells have been shown to have key functions in several mouse autoimmune disease models including experimental autoimmune encephalomyelitis (EAE) and collagen-induced arthritis (CIA). In addition, Th17 cells or their products have been shown to be associated with the pathology of a variety of human inflammatory and autoimmune disorders including multiple sclerosis, rheumatoid arthritis, psoriasis, Crohn's disease and asthma (Jetten (2009) Nucl. Recept. Signal. 7:e003; Manel et al. (2008) Nat. Immunol. 9:641-649). The pathogenesis of chronic autoimmune diseases including multiple sclerosis and rheumatoid arthritis arises from the break in tolerance towards self-antigens and the development of auto-aggressive effector T cells infiltrating the target tissues. Studies have shown that Th17 cells are one of the important drivers of the inflammatory process in tissue-specific autoimmunity (Steinman (2008) J. Exp. Med. 205:1517-1522; Leung et al. (2010) Cell. Mol. Immunol. 7:182-189). There is evidence that Th17 cells are activated during the disease process and are responsible for recruiting other inflammatory cells types, especially neutrophils, to mediate pathology in the target tissues (Korn et al. (2009) Annu. Rev. Immunol. 27:485-517).

RORγt plays a critical role in the pathogenic responses of Th17 cells (Ivanov et al. (2006) Cell 126:1121-1133). RORγt deficient mice produce few Th17 cells. In addition, RORγt deficiency resulted in amelioration of EAE. Further support for the role of RORγt in the pathogenesis of autoimmune or inflammatory diseases can be found in the following references: Jetten & Joo (2006) Adv. Dev. Biol. 16:313-355; Meier et al. (2007) Immunity 26:643-654; Aloisi & Pujol-Borrell (2006) Nat. Rev. Immunol. 6:205-217; Jager et al. (2009) J. Immunol. 183:7169-7177; Serafini et al. (2004) Brain Pathol. 14:164-174; Magliozzi et al. (2007) Brain 130:1089-1104; Barnes (2008) Nat. Rev. Immunol. 8:183-192.

In light of the role RORγ plays in the pathogenesis of diseases, it is desirable to prepare compounds that modulate RORγ activity, which can be used in the treatment of diseases mediated by RORγ.

### SUMMARY OF THE INVENTION

The invention is directed to novel RORγ modulators and their use in the treatment of diseases mediated by RORγ. Specifically, the invention is directed to compounds according to Formula (I):

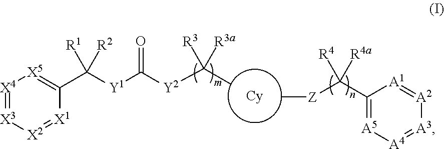

wherein:

m is 0, 1, or 2;

n is 0, 1, 2, or 3;

X¹, X², X³, X⁴, and X⁵ are each independently selected from N, N⁺—O⁻, CH, and CR⁵, wherein 0-3 of X¹, X², X³, X⁴, and X⁵ are N or N⁺—O⁻ and 1-3 of X¹, X², X³, X⁴, and X⁵ are CR⁵;

one of Y¹ and Y² is O or NR⁸ and the other is a bond;

or X¹ is CR⁵, Y¹ is NR⁸, Y² is a bond, and R⁵ and R⁸ taken together with the atoms to which they are attached form a five to seven membered ring, optionally containing an additional heteroatom selected from oxygen, nitrogen, and sulfur, which ring is optionally substituted by (C₁-C₄)alkyl;

Cy is (C₃-C₈)cycloalkyl, heterocycloalkyl, phenyl, or 5- or 6-membered heteroaryl, each of which is optionally substituted one, two, or three times, independently, by (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷—((C₀-C₃)alkyl)N((C₁-C₄)alkyl)C(O)R⁷, —((C₀-C₃)alkyl)CO₂R⁷, —((C₀-C₃)alkyl)CONR⁷R⁵, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;

Z is O, S, SO₂, C═O, NR⁶, or a bond;

A¹, A², A³, A⁴, and A⁵ are each independently selected from N, N+—O⁻, CH, and CR¹⁰, wherein 0-3 of A¹, A², A³, A⁴, and A⁵ are N or N, N+—O⁻ and 0-3 of A¹, A², A³, A⁴, and A⁵ are CR¹⁰;

R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁵;

R² is hydrogen, (C₁-C₆)alkyl, or (C₁-C₆)haloalkyl;

or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁵;

R³ and R^{3a} are each independently hydrogen, hydroxyl, (C₁-C₆)alkyl, (C₁-C₆)haloalkyl, halogen, (C₁-C₆)alkoxy, amino, (C₁-C₄)alkylamino, or ((C₁-C₄)alkyl)((C₁-C₄)alkyl)amino;

each R⁴ is independently selected from hydrogen, halogen, (C₁-C₆)alkyl, (C₁-C₆)haloalkyl, —CO₂R⁷, —CONR⁷R⁸, —OR⁹, and —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹;

each R^{4a} is independently selected from hydrogen, halogen, hydroxyl, amino, and (C₁-C₆)alkyl;

or R⁴ and R^{4a} taken together with the carbon atom to which they are attached form a three to eight membered ring, optionally containing a heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂R⁷, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

each R⁵ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁶ is hydrogen, (C₁-C₆)alkyl, (C₁-C₆)haloalkyl, (C₃-C₆)cycloalkyl, hydroxy(C₁-C₆)alkyl, (C₁-C₄)alkoxy(C₁-C₆)alkyl, —((C₀-C₃)alkyl)CO₂R⁷, —((C₀-C₃)alkyl)CONR⁷R⁸, aryl, heteroaryl, aryl(C₁-C₆)alkyl, heteroaryl(C₁-C₆)alkyl, or heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl;

or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;

R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by —CO₂R⁷, —CONH₂, —CONH(C₁-C₄)alkyl, —CON((C₁-C₄)alkyl)((C₁-C₄)alkyl), hydroxyl, (C₁-C₄)alkoxy, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

or R⁸ and R⁹ taken together with the nitrogen atom to which they are attached form a four to eight membered ring, optionally containing an additional heteroatom selected from oxygen, nitrogen, and sulfur, which ring is optionally substituted by cyano, (C₁-C₄)alkyl, (C₁-C₄)haloalkyl, (C₃-C₄)cycloalkyl, —CO₂H, —CO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷; and

R¹⁰ is (C₁-C₆)alkyl, (C₁-C₆)haloalkyl, (C₀-C₃)cycloalkyl, halogen, cyano, hydroxyl, hydroxy(C₁-C₆)alkyl, (C₁-C₆)alkoxy, (C₁-C₄)alkoxy(C₁-C₆)alkyl, —((C₀-C₃)alkyl)CO₂R⁷, —((C₀-C₃)alkyl)CONR⁷R⁸, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;

or a salt thereof, particularly, a pharmaceutically acceptable salt thereof.

In another aspect, this invention provides a pharmaceutical composition comprising a compound of Formula (I), or a pharmaceutically acceptable salt thereof, and a pharmaceutically acceptable excipient.

In another aspect, this invention provides for the use of a compound of Formula (I) or a pharmaceutically acceptable salt thereof for the treatment of diseases mediated by RORγ. The invention further provides for the use of a compound of Formula (I) or a pharmaceutically acceptable salt thereof as an active therapeutic substance in the treatment of a disease mediated by RORγ.

In another aspect, the invention provides a compound of Formula (I) or a pharmaceutically acceptable salt thereof for use in therapy.

In another aspect, the invention provides the use of a compound of Formula (I) or a pharmaceutically acceptable salt thereof in the manufacture of a medicament for the treatment of diseases mediated by RORγ.

Examples of such diseases for which compounds of Formula (I) may be used include autoimmune or inflammatory diseases such as multiple sclerosis, rheumatoid arthritis, psoriasis, uveitis, dry eye, glomerulonephritis, Crohn's disease and asthma, especially psoriasis

In yet another aspect, the invention is directed to methods of treating such diseases for example by administering to a patient (e.g. human) in need thereof an effective amount of a compound of Formula (I) or a pharmaceutically acceptable salt thereof.

### DETAILED DESCRIPTION OF THE INVENTION

As used herein, the term “alkyl” represents a saturated, straight, or branched hydrocarbon moiety. The term “(C₁-C₆)alkyl” refers to an alkyl moiety containing from 1 to 6 carbon atoms.

Exemplary alkyls include, but are not limited to methyl, ethyl, n-propyl, isopropyl, n-butyl, isobutyl, s-butyl, t-butyl, pentyl, and hexyl. C₀alkyl means that no alkyl group is present in the moiety. Thus, —((C₀)alkyl)CONH₂ is equivalent to —CONH₂.

When the term “alkyl” is used in combination with other substituent groups, such as “haloalkyl”, “hydroxyalkyl”, “alkoxyalkyl”, “arylalkyl”, or “heteroarylalkyl”, the term “alkyl” is intended to encompass a divalent straight or branched-chain hydrocarbon radical. For example, “arylalkyl” is intended to mean the radical-alkylaryl, wherein the alkyl moiety thereof is a divalent straight or branched-chain carbon radical and the aryl moiety thereof is as defined herein, and is represented by, for example, the bonding arrangement present in a benzyl group (—CH₂-phenyl); “halo(C₁-C₄)alkyl” is intended to mean a radical having one or more halogen atoms, which may be the same or different, at one or more carbon atoms of an alkyl moiety containing from 1 to 4 carbon atoms, which is a straight or branched-chain carbon radical, and is represented by, for example, a trifluoromethyl group (—CF₃).

As used herein, the term “cycloalkyl” refers to a non-aromatic, saturated, cyclic hydrocarbon ring. The term “(C₃-C₈)cycloalkyl” refers to a non-aromatic cyclic hydrocarbon ring having from three to eight ring carbon atoms. Exemplary “(C₃-C₈)cycloalkyl” groups useful in the present invention include cyclopropyl, cyclobutyl, cyclopentyl, cyclohexyl, cycloheptyl, and cyclooctyl.

“Alkoxy” means an alkyl radical containing the specified number of carbon atoms attached through an oxygen linking atom. The term “(C₁-C₄)alkoxy” refers to a straight- or branched-chain hydrocarbon radical having at least 1 and up to 4 carbon atoms attached through an oxygen linking atom. Exemplary “(C₁-C₄)alkoxy” groups useful in the present invention include, but are not limited to, methoxy, ethoxy, n-propoxy, isopropoxy, n-butoxy, s-butoxy, and t-butoxy.

“Aryl” represents a group or moiety comprising an aromatic, monovalent monocyclic or bicyclic hydrocarbon radical containing from 6 to 10 carbon ring atoms, to which may be fused one or more cycloalkyl rings.

Generally, in the compounds of this invention, aryl is phenyl.

Heterocyclic groups may be heteroaryl or heterocycloalkyl groups.

“Heteroaryl” represents a group or moiety comprising an aromatic monovalent monocyclic or bicyclic radical, containing 5 to 10 ring atoms, including 1 to 4 heteroatoms independently selected from nitrogen, oxygen and sulfur. This term also encompasses bicyclic heterocyclic-aryl compounds containing an aryl ring moiety fused to a heterocycloalkyl ring moiety, containing 5 to 10 ring atoms, including 1 to 4 heteroatoms independently selected from nitrogen, oxygen and sulfur. Illustrative examples of heteroaryls useful in the present invention include, but are not limited to, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, triazinyl, benzofuranyl, isobenzofuryl, 2,3-dihydrobenzofuryl, 1,3-benzodioxolyl, dihydrobenzodioxinyl, benzothienyl, indolizinyl, indolyl, isoindolyl, dihydroindolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, imidazopyridinyl, pyrazolopyridinyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

Generally, the heteroaryl groups present in the compounds of this invention are 5-membered and/or 6-membered monocyclic heteroaryl groups. Selected 5-membered heteroaryl groups contain one nitrogen, oxygen, or sulfur ring heteroatom, and optionally contain 1, 2, or 3 additional nitrogen ring atoms. Selected 6-membered heteroaryl groups contain 1, 2, or 3 nitrogen ring heteroatoms. Illustrative examples of 5- or 6-membered heteroaryl groups useful in the present invention include, but are not limited to furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl.

“Heterocycloalkyl” represents a group or moiety comprising a non-aromatic, monovalent monocyclic or bicyclic radical, which is saturated or partially unsaturated, containing 3 to 10 ring atoms, which includes 1 to 3 heteroatoms independently selected from nitrogen, oxygen and sulfur. Illustrative examples of heterocycloalkyls useful in the present invention include, but are not limited to, azetidinyl, pyrrolidinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, morpholinyl, thiomorpholinyl, tetrahydropyranyl, dihydropyranyl, 1,3-dioxanyl, 1,4-dioxanyl, 1,3-oxathiolanyl, 1,3-oxathianyl, 1,3-dithianyl, hexahydro-1H-1,4-diazepinyl, azabicylo[3.2.1]octyl, azabicylo[3.3.1]nonyl, azabicylo[4.3.0]nonyl, oxabicylo[2.2.1]heptyl and 1,5,9-triazacyclododecyl.

Generally, in the compounds of this invention, heterocycloalkyl groups are 5-7 membered heterocycloalkyl groups, such as pyrrolidinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, morpholinyl, thiomorpholinyl, tetrahydropyranyl, dihydropyranyl, and hexahydro-1H-1,4-diazepinyl.

“Oxo” represents a double-bonded oxygen moiety; for example, if attached directly to a carbon atom forms a carbonyl moiety (C═O).

The terms “halogen” and “halo” represent chloro, fluoro, bromo, or iodo substituents. “Hydroxy” or “hydroxyl” is intended to mean the radical —OH.

“RORγ” refers to all isoforms encoded by the RORC gene which include RORγ1 and RORγt.

“RORγ modulator” refers to a chemical compound that inhibits, either directly or indirectly, the activity of RORγ. RORγ modulators include antagonists and inverse agonists of RORγ.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

As used herein, the term “compound(s) of the invention” means a compound of Formula (I) (as defined above) in any form, i.e., any salt or non-salt form (e.g., as a free acid or base form, or as a pharmaceutically acceptable salt thereof) and any physical form thereof (e.g., including non-solid forms (e.g., liquid or semi-solid forms), and solid forms (e.g., amorphous or crystalline forms, specific polymorphic forms, solvates, including hydrates (e.g., mono-, di- and hemi-hydrates)), and mixtures of various forms.

As used herein, the term “optionally substituted” indicates that a group, such as alkyl, cycloalkyl, alkoxy, heterocycloalkyl, aryl, or heteroaryl, may be unsubstituted, or the group may be substituted with one or more substituent(s) as defined. In the case where groups may be selected from a number of alternative groups the selected groups may be the same or different.

The term “independently” means that where more than one substituent is selected from a number of possible substituents, those substituents may be the same or different. The alternative definitions for the various groups and substituent groups of Formula (I) provided throughout the specification are intended to particularly describe each compound species disclosed herein, individually, as well as groups of one or more compound species. The scope of this invention includes any combination of these group and substituent group definitions.

Suitably, m is 0, 1, or 2. In a specific embodiment of this invention, m is 1.

Suitably, n is 0, 1, 2, or 3. In another embodiment of this invention, n is 1 or 2.

Suitably, X¹, X², X³, X⁴, and X⁵ are each independently selected from N, N⁺—O⁻ (i.e. N-oxide), CH, and CR⁵, wherein 0-3 of X¹, X², X³, X⁴, and X⁵ are N⁺—O⁻ and 0-3 of X¹, X², X³, X⁴, and X⁵ are CR⁵. In another embodiment of this invention, X¹, X², X³, X⁴, and X⁵ are each independently selected from N⁺—O⁻, CH, and CR⁵, wherein 0-2 of X¹, X², X³, X⁴, and X⁵ are N or N⁺—O⁻ and 0-3 of X¹, X², X³, X⁴, and X⁵ are CR⁵. In another embodiment of this invention, X¹ and X⁵ are each independently selected from N, N⁺—O⁻, CH, and CR⁵, and X², X³, and X⁴ are each independently selected from CH and CR. In another embodiment of this invention, X¹ and X⁵ are each independently selected from N⁺—O⁻, CH, and CR⁵, and X², X³, and X⁴ are each independently selected from CH and CR⁵, wherein at least one of X¹ and X⁵ is N⁺—O⁻ and 0-3 of X¹, X², X³, X⁴, and X⁵ are CR⁵. In another embodiment of this invention, X¹ and X⁵ are each independently selected from N, N⁺—O⁻, and a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino (i.e. N⁺—O⁻, CH, and CR⁵, wherein R⁵ is halogen, cyano, (C₁-C₄)alkyl, (C₁-C₄)haloalkyl, (C₁-C₄)alkoxy, or ((C₁-C₄)alkyl)((C₁-C₄)alkyl)amino), and X², X³, and X⁴ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino (i.e. CH or CR⁵, wherein R⁵ is halogen, cyano, (C₁-C₄)alkyl, (C₁-C₄)haloalkyl, (C₁-C₄)alkoxy, or ((C₁-C₄)alkyl)((C₁-C₄)alkyl)amino), wherein at least one of X¹ and X¹ is N or N⁺—O⁻ and 2-4 of X¹, X², X³, X⁴, and X⁵ are a carbon atom substituted by hydrogen (i.e. CH). In another embodiment of this invention, X² is N or N⁺—O⁻, and X¹, X³, X⁴, and X⁵ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino, wherein 2-4 of X¹, X³, X⁴, and X⁵ are a carbon atom substituted by hydrogen. In another embodiment of this invention, X¹, X², X³, X⁴, and X⁵ are each independently selected from CH and CR⁵, wherein 0-3 of X¹, X², X³, X⁴, and X⁵ are CR⁵. In another embodiment of this invention, X¹, X², X³, X⁴, and X⁵ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino, wherein 2-5 of X¹, X², X³, X⁴, and X⁵ are a carbon atom substituted by hydrogen. In another embodiment of this invention, X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 2-4 of X², X³, X⁴, and X⁵ are a carbon atom substituted by hydrogen.

Suitably, one of Y¹ and Y² is O or NR⁸ and the other is a bond. In another embodiment of this invention, one of Y¹ and Y² is O, NH, or N((C₁-C₄)alkyl) and the other is a bond. In a specific embodiment of this invention, Y¹ is NH or NCH₃ and Y² is a bond. In another specific embodiment of this invention, Y¹ is NH and Y² is a bond. In another specific embodiment of this invention, Y¹ is a bond and Y² is NH.

In another embodiment of this invention, X¹ is CR⁵, Y¹ is NR⁸, Y² is a bond, and R⁵ and R⁸ taken together with the atoms to which they are attached form a five to seven membered ring, optionally containing an additional heteroatom selected from oxygen, nitrogen, and sulfur, which ring is optionally substituted by (C₁-C₄)alkyl. In another embodiment of this invention, X¹ is CR⁵, Y¹ is NR⁸, Y² is a bond, and R⁵ and R⁸ taken together represent —CH₂—, —CH₂CH₂—, or —CH₂CH₂CH₂—.

Suitably, Cy is (C₃-C₈)cycloalkyl, heterocycloalkyl, phenyl, or 5- or 6-membered heteroaryl, each of which is optionally substituted one, two, or three times, independently, by (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl. In another embodiment of this invention, Cy is heterocycloalkyl, phenyl, or 5- or 6-membered heteroaryl, each of which is optionally substituted one or two times, independently, by (C₁-C₆)alkyl, (C₁-C₆)haloalkyl, halogen, cyano, (C₁-C₄)alkoxy, ((C₁-C₄)alkyl)amino((C₁-C₄)alkyl)((C₁-C₄)alkyl)amino, —((C₀-C₃)alkyl)COR⁷, or —((C₀-C₃)alkyl)CONR⁷R⁸. In another embodiment of this invention, Cy is (C₃-C₆)cycloalkyl, azetidinyl, pyrrolidinyl, pyrazolidinyl, pyrazolinyl, imidazolidinyl, imidazolinyl, oxazolinyl, thiazolinyl, tetrahydrofuranyl, dihydrofuranyl, piperidinyl, piperazinyl, morpholinyl, thiomorpholinyl, tetrahydropyranyl, dihydropyranyl, dioxanyl, oxathianyl, phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, each of which is optionally substituted one, two, or three times, independently, by (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl. In another embodiment of this invention, Cy is piperidinyl, piperazinyl, phenyl, pyridinyl, pyridazinyl, pyrazinyl, or pyrimidinyl, each of which is optionally substituted one, two, or three times, independently, by (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl. In another embodiment of this invention, Cy is piperidinyl, piperazinyl, phenyl, pyridinyl, pyridazinyl, pyrazinyl, or pyrimidinyl, each of which is optionally substituted one or two times, independently, by (C₁-C₆)alkyl, (C₁-C₆)haloalkyl, halogen, cyano, (C₁-C₄)alkoxy, (C₁-C₄)alkyl)((C₁-C₄)alkyl)amino, —((C₀-C₃)alkyl)CO₂H, —((C₀-C₃)alkyl)CO₂(C₁-C₆)alkyl, or —((C₀-C₃)alkyl)CONH(C₁-C₆)alkyl. In another embodiment of this invention, Cy is phenyl, which is optionally substituted one, two, or three times, independently, by (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)CO₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl. In another embodiment of this invention, Cy is phenyl, which is optionally substituted one or two times, independently, by halogen, (C₁-C₄)alkyl, (C₁-C₄)haloalkyl, cyano, (C₁-C₄)alkoxy, —((C₀-C₃)alkyl)CO₂R⁷, or —((C₀-C₃)alkyl)CONR⁷R⁸ or ((C₁-C₄)alkyl)((C₁-C₄)alkyl)amino. In another embodiment of this invention, Cy is phenyl, which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino. In a specific embodiment of this invention, Cy is phenyl.

Suitably, Z is O, S, SO₂, C═O, NR⁶, or a bond. In another embodiment of this invention, Z is O, NR⁶, or a bond. In another embodiment of this invention, Z is O, NH, —N(C₁-C₄)alkyl, —N((C₀-C₃)alkyl)CO₂R⁷, —N((C₀-C₃)alkyl)CONR⁷R⁸ or a bond. In another embodiment of this invention, Z is a bond, O, or NH. In another embodiment of this invention, Z is O or NH. In a specific embodiment of this invention, Z is O.

Suitably, A¹, A², A³, A⁴, and A⁵ are each independently selected from N⁺—O⁻, CH, and CR¹⁰, wherein 0-3 of A¹, A², A³, A⁴, and A⁵ are N or N⁺—O⁻ and 0-3 of A¹, A², A³, A⁴, and A⁵ are CR¹⁰. In another embodiment of this invention, A¹, A², A³, A⁴, and A⁵ are each independently selected from N⁺—O⁻, and a carbon atom substituted by hydrogen, halogen, cyano, (C₁-C₄)alkyl, (C₁-C₄)haloalkyl, hydroxyl, (C₁-C₄)alkoxy, or ((C₁-C₄)alkyl)((C₁-C₄)alkyl)amino, wherein 1-2 of A¹, A², A³, A⁴, and A⁵ are N or N⁺—O⁻ and 1-4 of A¹, A², A³, A⁴, and A⁵ are a carbon atom substituted by hydrogen. In another embodiment of this invention, A² and A⁴ are each independently selected from N, N⁺—O⁻, and a carbon atom substituted by hydrogen, halogen, cyano, (C₁-C₄)alkyl, hydroxyl, (C₁-C₄)alkoxy, or ((C₁-C₄)alkyl)((C₁-C₄)alkyl)amino, and A¹, A³, and A⁵ are each independently a carbon atom substituted by hydrogen, halogen, (C₁-C₄)alkyl, hydroxyl, (C₁-C₄)alkoxy, or ((C₁-C₄)alkyl)((C₁-C₄)alkyl)amino, wherein at least one of A² and A⁴ is N⁺—O⁻. In another embodiment of this invention, A² and A⁴ are each independently selected from N, N⁺—O⁻, CH, and C((C₁-C₄)alkyl), and A¹, A³, and A⁵ are each independently selected from CH and C((C₁-C₄)alkyl), wherein at least one of A² and A⁴ is N or N⁺—O⁻.

Suitably, R⁷ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁵. In another embodiment of this invention, R⁷ is (C₃-C₆)alkyl, (C₃-C₈)cycloalkyl, (C₁-C₆)alkoxy(C₁-C₂)alkyl, aryl, or heteroaryl, each of which is optionally substituted one, two, or three times, independently, by R⁵. In another embodiment of this invention, R¹ is (C₃-C₆)alkyl, (C₃-C₆)cycloalkyl, (C₁-C₆)alkoxy(C₁-C₂)alkyl, phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino (i.e. wherein R⁵ is halogen, (C₁-C₄)alkyl, (C₁-C₄)haloalkyl, cyano, (C₁-C₄)alkoxy, or ((C₁-C₄)alkyl(C₁-C₄)alkyl)amino). In another embodiment of this invention, R¹ is (C₃-C₆)alkyl, (C₃-C₆)cycloalkyl, phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl is optionally substituted one or two times, independently, by halogen, (C₁-C₄)alkyl, (C₁-C₄)alkoxy, or ((C₁-C₄)alkyl)((C₁-C₄)alkyl)amino. In another embodiment of this invention, R⁷ is (C₃-C₆)alkyl. In another embodiment of this invention, R¹ is (C₅-C₆)alkyl. In another embodiment of this invention, R¹ is phenyl or pyridinyl, each of which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino. In another embodiment of this invention, R¹ is phenyl or pyridinyl, each of which is optionally substituted one or two times, independently, by halogen, (C₁-C₄)alkyl, (C₁-C₄)alkoxy, or ((C₁-C₄)alkyl)((C₁-C₄)alkyl)amino. In another embodiment of this invention, R⁷ is phenyl optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino. In a specific embodiment of this invention, R¹ is phenyl or pyridinyl. In another specific embodiment of this invention, R¹ is phenyl.

Suitably, R² is hydrogen, (C₁-C₆)alkyl, or (C₁-C₆)haloalkyl. In another embodiment of this invention, hydrogen or (C₁-C₄)alkyl. In another embodiment of this invention, R² is hydrogen or methyl. In a specific embodiment of this invention, R² is hydrogen.

In another embodiment of this invention, R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁵. In another embodiment of this invention, R¹ and R² taken together represent —CH₂CH₂CH₂—, —CH₂CH₂CH₂CH₂—, or —CH₂CH₂CH₂CH₂CH₂—.

Suitably, R³ and R^{3a} are each independently hydrogen, hydroxyl, (C₁-C₆)alkyl, (C₁-C₆)haloalkyl, halogen, (C₁-C₆)alkoxy, amino, (C₁-C₄)alkylamino, or ((C₁-C₄)alkyl)((C₁-C₄)alkyl)amino. In another embodiment of this invention, R³ and R^{3a} are each independently hydrogen or methyl. In a specific embodiment of this invention, R³ and R^{3a} are each independently hydrogen.

Suitably, each R⁴ is independently selected from hydrogen, halogen, (C₁-C₆)alkyl, (C₁-C₆)haloalkyl, —CO₂R⁷, —CONR⁷R⁸, —OR⁹, and —NR⁶R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹. In another embodiment of this invention, each R⁴ is independently selected from hydrogen, (C₁-C₄)alkyl, (C₁-C₄)haloalkyl, —OR⁹, and —NR⁸R⁹, wherein said (C₁-C₄)alkyl or (C₁-C₄)haloalkyl is optionally substituted by hydroxyl, —OR⁹, —CO₂R⁷, —CONR⁷R⁸, or —NR⁸R⁹. In another embodiment of this invention, each R⁴ is independently selected from hydrogen, halogen, (C₁-C₄)alkyl, (C₁-C₄)alkylamino, ((C₁-C₄)alkyl)((C₁-C₄)alkyl)amino, (C₁-C₄)alkoxy(C₁-C₄)alkylamino, —NHCO₂(C₁-C₄)alkyl, (C₁-C₄)alkoxy, hydroxy(C₂-C₄)alkoxy, (C₁-C₄)alkoxy(C₂-C₄)alkoxy, amino(C₂-C₄)alkoxy, —O((C₁-C₄)alkyl)CO₂R⁷, —O((C₁-C₄)alkyl)CONH₂, —O((C₁-C₄)alkyl)CONH(C₁-C₄)alkyl, —O((C₁-C₄)alkyl)CON((C₁-C₄)alkyl)((C₁-C₄)alkyl), and CO₂R⁷. In another embodiment of this invention, each R⁴ is independently selected from hydrogen, halogen, (C₁-C₄)alkyl, (C₁-C₄)alkylamino, ((C₁-C₄)alkyl)((C₁-C₄)alkyl)amino, (C₁-C₄)alkoxy(C₁-C₄)alkylamino, (C₁-C₄)alkoxy, hydroxy(C₂-C₄)alkoxy, (C₁-C₄)alkoxy(C₂-C₄)alkoxy, amino(C₂-C₄)alkoxy, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₄)alkyl, and —O((C₁-C₃)alkyl)CON((C₁-C₄)alkyl)((C₁-C₄)alkyl). In another embodiment of this invention, each R⁴ is independently selected from hydrogen, (C₁-C₄)alkyl, (C₁-C₄)alkoxy, hydroxy(C₂-C₄)alkoxy, (C₁-C₄)alkoxy(C₂-C₄)alkoxy, amino(C₂-C₄)alkoxy, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₄)alkyl, and —O((C₁-C₃)alkyl)CON((C₁-C₄)alkyl)((C₁-C₄)alkyl). In another embodiment of this invention, each R⁴ is independently selected from (C₁-C₄)alkoxy, hydroxy(C₂-C₄)alkoxy, (C₁-C₄)alkoxy(C₂-C₄)alkoxy, amino(C₂-C₄)alkoxy, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₄)alkyl, and —O((C₁-C₃)alkyl)CON((C₁-C₄)alkyl)((C₁-C₄)alkyl). In another embodiment of this invention, each R⁴ is independently selected from (C₁-C₄)alkoxy, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₄)alkyl, and —O((C₁-C₃)alkyl)CON((C₁-C₄)alkyl)((C₁-C₄)alkyl). In another embodiment of this invention, each R⁴ is independently selected from (C₁-C₄)alkyl and (C₁-C₄)alkoxy. In a specific embodiment of this invention, each R⁴ is hydrogen.

Suitably, each R^{4a} is independently selected from hydrogen, halogen, hydroxyl, amino, and (C₁-C₆)alkyl. In another embodiment of this invention, each R⁴ is independently selected from hydrogen, halogen, and (C₁-C₄)alkyl. In another embodiment of this invention, each R^{4a} is independently selected from is hydrogen, fluorine, and methyl. In another embodiment of this invention, each R⁴ is independently selected from is hydrogen and methyl. In a specific embodiment of this invention, each R^{4a} is hydrogen. In a specific embodiment of this invention, each R^{4a} is methyl.

In another embodiment of this invention. R⁴ and R^{4a} taken together with the carbon atom to which they are attached form a three to eight membered ring, optionally containing a heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂R⁷, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷. In another embodiment of this invention, R⁴ and R^{4a} taken together represent —CH₂CH₂—, —CH₂CH₂CH₂—, —CH₂CH₂CH₂CH₂—, or —CH₂CH₂CH₂CH₂CH₂—.

One particular embodiment of the invention is a compound of Formula (Ia):

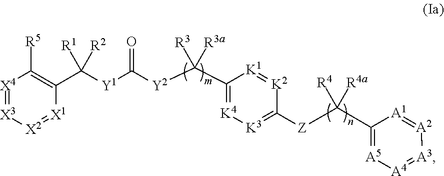

wherein:

m is 1;

n is 1 or 2;

X¹, X², X³, and X⁴ are each independently selected from N, N⁺—O⁻, CH, and CR⁵, wherein 0-2 of X¹, X², X³, and X⁴ are N or N⁺—O⁻ and 0-2 X¹, X², X³, and X⁴ are CR⁵;

Y¹ is NH or NCH₃ and Y² is a bond;

K¹, K², K³, and K⁴ are each independently selected from N, N⁺—O⁻, CH, and CR¹⁰, wherein 0-2 of K¹, K², K³, and K⁴ are N or N⁺—O⁻ and 0-2 of K¹, K², K³, and K⁴ are CR¹⁰;

Z is O, NR⁶, or a bond;

A¹, A², A³, A⁴, and A⁵ are each independently selected from N⁺—O⁻, CH, and CR¹⁰, wherein 0-3 of A¹, A², A³, A⁴, and A⁵ are N or N⁺—O⁻ and 0-3 of A¹, A², A³, A⁴, and A⁵ are CR¹⁰;

R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁵;

R² is hydrogen, (C₁-C₆)alkyl, or (C₁-C₆)haloalkyl;

or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁵;

R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl)((C₁-C₄)alkyl)amino;

each R⁴ is independently selected from hydrogen, halogen, (C₁-C₄)alkyl, (C₁-C₄)haloalkyl, —OR⁹, and —NR⁸R⁹, wherein said (C₁-C₄)alkyl or (C₁-C₄)haloalkyl is optionally substituted by hydroxyl, —OR⁹, —CO₂R⁷, —CONR⁷R⁸, or —NR⁸R⁹

each R^{4a} is independently selected from hydrogen, halogen, hydroxyl, amino, and (C₁-C₄)alkyl;

each R⁵ is independently selected from (C₁-C₆)alkyl, (C₁-C₆)haloalkyl, (C₃-C₈)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁶ is hydrogen, (C₁-C₆)alkyl, (C₁-C₆)haloalkyl, (C₃-C₆)cycloalkyl, hydroxy(C₁-C₆)alkyl, (C₁-C₄)alkoxy(C₁-C₆)alkyl, —((C₀-C₃)alkyl)CO₂R⁷, —((C₀-C₃)alkyl)CONR⁷R⁸, aryl, heteroaryl, aryl(C₁-C₆)alkyl, heteroaryl(C₁-C₆)alkyl, or heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;

R⁸ is hydrogen, (C₁-C₆)alkyl, or (C₁-C₆)haloalkyl;

or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;

R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by —CO₂R⁷, —CONH₂, —CONH(C₁-C₄)alkyl, —CON((C₁-C₄)alkyl)((C₁-C₄)alkyl), hydroxyl, (C₁-C₄)alkoxy, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;

or R⁸ and R⁹ taken together with the nitrogen atom to which they are attached form a four to eight membered ring, optionally containing an additional heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂H, —CO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R′, —NHC(O)R⁷, or —N((C₁-C₄)alkyl)C(O)R⁷; and

R¹⁰ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, —((C₀-C₃)alkyl)CO₂R⁷, —((C₀-C₃)alkyl)CONR⁷R⁸, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;

or a pharmaceutically acceptable salt thereof.

Another particular embodiment of the invention is a compound of Formula (Ia) wherein:

m is 1;

n is 1 or 2;

X¹, X², X³, and X⁴ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino, wherein 2-4 of X¹, X², X³, and X⁴ are a carbon atom substituted by hydrogen;

Y¹ is NH or NCH₃ and Y² is a bond;

K¹, K², K³, and K⁴ are each independently a carbon atom substituted by hydrogen, halogen, (C₁-C₄)alkyl, (C₁-C₄)alkoxy, or ((C₁-C₄)alkyl)((C₁-C₄)alkyl)amino, wherein 2-4 of K¹, K², K³, and K⁴ are a carbon atom substituted by hydrogen;

Z is O, NH, —N(C₁-C₄)alkyl, —N((C₀-C₃)alkyl)CO₂R⁷, —N((C₀-C₃)alkyl)CONR⁷R⁸, or a bond;

A² and A⁴ are each independently selected from N, N⁺—O⁻, CH, and C((C₁-C₄)alkyl), and A¹, A³, and A⁵ are each independently selected from CH and C((C₁-C₄)alkyl), wherein at least one of A² and A⁴ is N or N⁺—O⁻;

R¹ is (C₃-C₆)alkyl, (C₃-C₆)haloalkyl, (C₃-C₆)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁵;

R² is hydrogen;

R³ and R^{3a} are each independently hydrogen or methyl;

each R⁴ is independently selected from hydrogen, (C₁-C₄)alkyl, (C₁-C₄)alkoxy, hydroxy(C₂-C₄)alkoxy, (C₁-C₄)alkylamino, ((C₁-C₄)alkyl)((C₁-C₄)alkyl)amino, (C₁-C₄)alkoxy(C₁-C₄)alkylamino, (C₁-C₄)alkoxy(C₂-C₄)alkoxy, amino(C₂-C₄)alkoxy, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₄)alkyl, and —O((C₁-C₃)alkyl)CON((C₁-C₄)alkyl)((C₁-C₄)alkyl);

each R^{4a} is independently selected from hydrogen, hydroxyl, amino, and (C₁-C₄)alkyl;

each R⁵ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;

R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl; and

R⁸ is hydrogen, (C₁-C₆)alkyl, or (C₁-C₆)haloalkyl;

or a pharmaceutically acceptable salt thereof.

Another particular embodiment of the invention is a compound of Formula (Ia) wherein:

m is 1;

n is 1 or 2;

X¹. X², X³, and X⁴ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino, wherein 2-4 of X¹, X², X³, and X⁴ are a carbon atom substituted by hydrogen;

Y¹ is NH and Y² is a bond;

K¹, K², K³, and K⁴ are each independently a carbon atom substituted by hydrogen, halogen, (C₁-C₄)alkyl, (C₁-C₄)alkoxy, or ((C₁-C₄)alkyl)((C₁-C₄)alkyl)amino, wherein 2-4 of K¹, K², K³, and K⁴ are a carbon atom substituted by hydrogen;

Z is O, NH, —N(C₁-C₄)alkyl, or a bond;

A² and A⁴ are each independently selected from N, N⁺—O⁻, CH, and C((C₁-C₄)alkyl), and A¹, A³, and A⁵ are each independently selected from CH and C((C₁-C₄)alkyl), wherein at least one of A² and A⁴ is N or N⁺—O⁻;

R¹ is phenyl optionally substituted one or two times, independently, by halogen, (C₁-C₄)alkyl, (C₁-C₄)haloalkyl, cyano, (C₁-C₄)alkoxy, or ((C₁-C₄)alkyl(C₁-C₄)alkyl)amino;

R² is hydrogen;

R³ and R^{3a} are each independently hydrogen or methyl;

each R⁴ is independently selected from hydrogen, (C₁-C₄)alkyl, (C₁-C₄)alkylamino, ((C₁-C₄)alkyl)((C₁-C₄)alkyl)amino, and (C₁-C₄)alkoxy; and

each R^{4a} is independently selected from hydrogen, hydroxyl, amino, and (C₁-C₄)alkyl;

or a pharmaceutically acceptable salt thereof.

Specific compounds of this invention include:
N-((2-chloro-4-methylphenyl)(phenyl)methyl)-2-(4-(pyridin-3-ylmethoxy)phenyl)acetamide;
N-((4-chloro-3-methylphenyl)(phenyl)methyl)-2-(4-((2-methylpyridin-3-yl)methoxy)phenyl)acetamide;
N-((2,4-dimethylphenyl)(pyridin-2-yl)methyl)-2-(4-((2-methylpyridin-3-yl)methoxy)phenyl)acetamide;
N-((4-chloro-2-methylphenyl)(phenyl)methyl)-2-(4-((2,4-dimethylpyridin-3-yl)methoxy)phenyl)acetamide;
N-((2-chloro-4-methylphenyl)(phenyl)methyl)-2-(4-(pyrimidin-5-ylmethoxy)phenyl)acetamide;
2-(4-(benzyloxy)phenyl)-N-((4-chlorophenyl)(phenyl)methyl)acetamide;
N-(1-(4-chloro-2-methylphenyl)-3-methylbutyl)-2-(4-((2-methylpyridin-3-yl)methoxy)phenyl)acetamide;
N-((2,4-dimethylphenyl)(phenyl)methyl)-2-(4-(2-(2-methylpyridin-3-yl)ethoxy)phenyl)acetamide;
N-((2,4-dichloro-5-fluorophenyl)(phenyl)methyl)-2-(4-((2-methylpyridin-3-yl)methoxy)phenyl)acetamide;
N-((2,4-dimethylphenyl)(phenyl)methyl)-2-(4-(((2-methylpyridin-3-yl)methyl)amino)phenyl)acetamide;
N-((4-chloro-2-methylphenyl)(1-methyl-1H-pyrazol-4-yl)methyl)-2-(4-((2-methylpyridin-3-yl)methoxy)phenyl)acetamide;
2-(2,4-dimethylphenyl)-N-(4-((2-methylpyridin-3-yl)methoxy)benzyl)-2-phenyl)acetamide;
N-((4-chloro-2-methylphenyl)(phenyl)methyl)-2-(4-((2-methylpyridin-3-yl)oxy)phenyl)acetamide;
N-((2,4-dimethylphenyl)(phenyl)methyl)-2-(3-methyl-4-((2-methylpyridin-3-yl)methoxy)phenyl)acetamide;
N-((4-chloro-2-methylphenyl)(phenyl)methyl)-2-(4-(((2-methylpyridin-3-yl)methyl)amino)piperidin-1-yl)acetamide;
N-((2,4-dimethylphenyl)(phenyl)methyl)-2-(4-methyl-4-(((2-methylpyridin-3-yl)methyl)amino)piperidin-1-yl)acetamide;
N-((2,4-dimethylphenyl)(phenyl)methyl)-2-(4-(2-hydroxy-2-(2-methylpyridin-3-yl)ethoxy)phenyl)acetamide;
N-((2-chloro-4-methylphenyl)(phenyl)methyl)-2-(4-(pyridin-3-ylmethoxy)phenyl)acetamide;
N-((2,4-dimethylphenyl)(phenyl)methyl)-2-(4-(pyridin-3-ylmethoxy)phenyl) acetamide;
N-((4-chloro-2-methylphenyl)(phenyl)methyl)-2-(4-(pyridin-3-ylmethoxy)phenyl)acetamide;
2-(4-((2-methylpyridin-3-yl)methoxy)phenyl)-N-(pyridin-3-yl(p-tolyl) methyl)acetamide;
N-(1-(4-chlorophenyl)-4-methylpentyl)-2-(4-((2-methylpyridin-3-yl)methoxy)phenyl)acetamide;
N-((3,5-dimethylpyridin-2-yl)(phenyl)-methyl)-2-(4-((2-methylpyridin-3-yl)methoxy)phenyl)acetamide;
N-((3,4-dimethylphenyl)(phenyl)methyl)-2-(4-((2-methylpyridin-3-yl)methoxy)phenyl)acetamide;
N-((2,4-dimethylphenyl)(phenyl)methyl)-2-(4-((2-methylpyridin-3-yl)methoxy)phenyl)acetamide;
N-((2,4-dimethylphenyl)(phenyl)methyl)-2-(4-((6-methylpyridin-3-yl)methoxy)phenyl)acetamide;
N-((4-chloro-2-methylphenyl)(phenyl)-methyl)-2-(4-((2-methylpyridin-3-yl)methoxy)phenyl)acetamide;
N-((4-chloro-2-methylphenyl)(phenyl)-methyl)-2-(4-((6-methylpyridin-3-yl)methoxy)phenyl)acetamide;
N-((2,4-dichlorophenyl)(phenyl)methyl)-2-(4-((2-methylpyridin-3-yl)methoxy)phenyl)acetamide;
N-((2,4-dimethylphenyl)(phenyl)methyl)-2-(4-(pyridin-4-ylmethoxy)phenyl)acetamide;
N-((2,4-dimethylphenyl)(phenyl)methyl)-2-(4-((3-methylpyridin-4-yl)methoxy)phenyl)acetamide;
N-((2,4-dimethylphenyl)(phenyl)methyl)-2-(4-(1-hydroxy-2-(2-methylpyridin-3-yl)ethyl)phenyl)acetamide;
N-(1-(3,5-dimethylpyridin-2-yl)-4-methylpentyl)-2-(4-(1-hydroxy-2-(2-methylpyridin-3-yl)ethyl)phenyl)acetamide;
2-(4-(1-(2-amino-2-oxoethoxy)-2-(2-methylpyridin-3-yl)ethyl)phenyl)-N-((2,4-dimethylphenyl)(phenyl)methyl)acetamide;
N-((4-chloro-2-methylphenyl)(phenyl)methyl)-2-(4-(2-(4,6-dihydroxypyrimidin-5-yl)ethoxy)phenyl)acetamide;
N-((4-chloro-2-methylphenyl)(phenyl)methyl)-2-(4-(2-(4-chloro-6-hydroxypyrimidin-5-yl)ethoxy)phenyl)acetamide;
N-((4-chloro-2-methylphenyl)(phenyl)methyl)-2-(4-(2-(pyrimidin-5-yl)ethoxy)phenyl)acetamide;
N-((4-chloro-2-methylphenyl)(phenyl)methyl)-2-(2-(((2-methylpyridin-3-yl)methyl)amino)thiazol-4-yl)acetamide;
N-((4-chloro-2-methylphenyl)(phenyl)methyl)-2-(3-methyl-4-((2-methylpyridin-3-yl)methoxy)phenyl)acetamide;
N-(1-(4-chlorophenyl)-2-methylpropyl)-2-(4-((2-methylpyridin-3-yl)methoxy)phenyl)acetamide;
2-(4-((2-methylpyridin-3-yl)methoxy)phenyl)-N-(4-(p-tolyl)tetrahydro-2H-pyran-4-yl)acetamide;
N-((2,4-dimethylphenyl)(thiazol-5-yl)methyl)-2-(4-((2-methylpyridin-3-yl)methoxy)phenyl)acetamide;
N-((5-methylpyridin-2-yl)(phenyl)methyl)-2-(4-((2-methylpyridin-3-yl)methoxy)phenyl)acetamide;
N-(1-(2,4-dimethylphenyl)-4-methylpentyl)-2-(4-(1-hydroxy-2-(2-methylpyridin-3-yl)ethyl)phenyl)acetamide;
N-(1-(3,5-dimethylpyridin-2-yl)-4-methylpentyl)-2-(4-((2-methylpyridin-3-yl)methoxy)phenyl)acetamide;
2-(1-(4-(2-(((2,4-dimethylphenyl)(phenyl)methyl)amino)-2-oxoethyl)phenyl)-2-(2-methylpyridin-3-yl)ethoxy)acetic acid;
N-((4-hydroxy-2-methylphenyl)(phenyl)methyl)-2-(4-((2-methylpyridin-3-yl)methoxy)phenyl)acetamide;
2-(4-((4-chlorobenzyl)oxy)phenyl)-N-((4-chlorophenyl)(phenyl)methyl)acetamide;

or a pharmaceutically acceptable salt thereof.

The meaning of any functional group or substituent thereon at any one occurrence in Formula (I), or any subformula thereof, is independent of its meaning, or any other functional group's or substituent's meaning, at any other occurrence, unless stated otherwise.

The compounds according to Formula (I) may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in Formula (I), or in any chemical structure illustrated herein, is not specified the structure is intended to encompass all individual stereoisomers and all mixtures thereof. Thus, compounds according to Formula (I) containing one or more chiral center may be used as racemic mixtures, enantiomerically enriched mixtures, or as enantiomerically pure individual stereoisomers.

Individual stereoisomers of a compound according to Formula (I) which contain one or more asymmetric centers may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

“Enantiomerically enriched” refers to products whose enantiomeric excess is greater than zero. For example, enantiomerically enriched refers to products whose enantiomeric excess is greater than 50% ee, greater than 75% ee, and greater than 90% ee.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that it constitutes 95% of the product, then the enantiomeric excess would be 90% cc (the amount of the enriched enantiomer, 95%, minus the amount of the other enantiomer, 5%).

“Enantiomerically pure” means products whose enantiomeric excess is 99% ee or greater.

When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

For solvates of the compounds of Formula (I), or salts thereof, that are in crystalline form, the skilled artisan will appreciate that pharmaceutically acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

Because of their potential use in medicine, the salts of the compounds of Formula (I) are preferably pharmaceutically acceptable. Suitable pharmaceutically acceptable salts include those described by Berge, Bighley and Monkhouse J. Pharm. Sci (1977) 66, pp 1-19. Salts encompassed within the term “pharmaceutically acceptable salts” refer to non-toxic salts of the compounds of Formula (I).

Salts of the compounds of Formula (I) containing a basic amine or other basic functional group may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, γ-hydroxybutyrates, glycolates, tartrates mandelates, and sulfonates, such as xylenesulfonates, methanesulfonates, propanesulfonates, naphthalene-1-sulfonates and naphthalene-2-sulfonates.

Salts of the compounds of Formula (I) containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, his-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

Other non-pharmaceutically acceptable salts, e.g. trifluoroacetate, may be used, for example in the isolation of compounds of the invention, and are included within the scope of this invention.

The invention includes within its scope all possible stoichiometric and non-stoichiometric forms of the salts of the compounds of Formula (I).

If a compound of Formula (I) containing a basic amine or other basic functional group is isolated as a salt, the corresponding free base form of that compound may be prepared by any suitable method known to the art, including treatment of the salt with an inorganic or organic base, suitably an inorganic or organic base having a higher pKₐ than the free base form of the compound. Similarly, if a compound of Formula (I) containing a carboxylic acid or other acidic functional group is isolated as a salt, the corresponding free acid form of that compound may be prepared by any suitable method known to the art, including treatment of the salt with an inorganic or organic acid, suitably an inorganic or organic acid having a lower pKₐ than the free acid form of the compound.

The invention also includes various deuterated forms of the compounds of Formula (I). Each available hydrogen atom attached to a carbon atom may be independently replaced with a deuterium atom. A person of ordinary skill in the art will know how to synthesize deuterated forms of the compounds of Formula (I). Commercially available deuterated starting materials may be employed in the preparation of deuterated forms of the compounds of Formula (I), or they may be synthesized using conventional techniques employing deuterated reagents (e.g. lithium aluminum deuteride or sodium borodeuteride).

### Methods of Use

Modulators of RORγ can be useful in the treatment of diseases mediated by RORγ, particularly autoimmune or inflammatory diseases and cancer. Such inflammatory or autoimmune diseases include multiple sclerosis, rheumatoid arthritis, psoriasis, Crohn's disease, inflammatory bowel disease, graft-versus-host disease (GVHD), Sjorgen's syndrome, optic neuritis, chronic obstructive pulmonary disease, asthma, type I diabetes, neuromyelitis optica, myasthenia gravis, uveitis, Behcets disease, Guillain-Barre syndrome, psoriatic arthritis, Graves' disease, allergic contact dermatitis, systemic lupus erythematosus, cutaneous lupus erythematosus, ankylosing spondylitis, Hashimoto Thyroiditis, dry eye and glomerulonephritis, myocarditis, especially psoriasis Such cancers include multiple myeloma and lytic bone disease associated with multiple myeloma, acute myelogenous leukemia (AML), head and neck squamous cell carcinoma, bladder carcinoma, gastric cancer, hepatocellular carcinoma, melanoma, medulloblastoma and colon cancer. Accordingly, in another aspect the invention is directed to methods of treating such diseases using a compound of Formula (I) or a pharmaceutically acceptable salt thereof. The methods of treatment of the invention comprise administering an effective amount of a compound according to Formula (I) or a pharmaceutically acceptable salt thereof to a patient (particularly a human) in need thereof.

In a further aspect, the invention is directed to a compound of Formula (I) or a pharmaceutically acceptable salt thereof for use in therapy. In particular, for use in the treatment of diseases mediated by RORγ, particularly autoimmune or inflammatory diseases and cancer, such as those disclosed above.

In a further aspect, the invention is directed to the use of a compound of Formula (I) or a pharmaceutically acceptable salt thereof in the manufacture of a medicament for the treatment of diseases mediated by RORγ, particularly autoimmune or inflammatory diseases and cancer, such as those disclosed above.

As used herein, “treatment” in reference to a condition means: (1) the amelioration or prevention of the condition being treated or one or more of the biological manifestations of the condition being treated, (2) the interference with (a) one or more points in the biological cascade that leads to or is responsible for the condition being treated or (b) one or more of the biological manifestations of the condition being treated, or (3) the alleviation of one or more of the symptoms or effects associated with the condition being treated.

As indicated above. “treatment” of a condition includes prevention of the condition. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

An “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, “patient” refers to a human or a mammal, especially a human.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the amount administered and the duration such regimens are administered, for a compound of the invention depend on the condition being treated, the severity of the condition being treated, the age and physical condition of the patient being treated, the medical history of the patient to be treated, the nature of concurrent therapy, the particular route of administration chosen, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change. Typical daily dosages range from 1 mg to 1000 mg.

It will be appreciated by those skilled in the art that certain protected derivatives of compounds of Formula (I), which may be made prior to a final deprotection stage, may not possess pharmacological activity as such, but may, in certain instances, be administered orally or parenterally and thereafter metabolized in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention.

Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Preferred “pro-moieties” for compounds of the invention include: ester, carbonate ester, hemi-ester, phosphate ester, nitro ester, sulfate ester, sulfoxide, amide, carbamate, azo-, phosphamide, glycoside, ether, acetal, and ketal derivatives of the compounds of Formula (I).

Administration of a compound of the invention as a prodrug may enable the skilled artisan to do one or more of the following: (a) modify the onset of the compound in vivo; (b) modify the duration of action of the compound in vivo; (c) modify the transportation or distribution of the compound in vivo; (d) modify the solubility of the compound in vivo; and (e) overcome or overcome a side effect or other difficulty encountered with the compound.

The invention further includes the use of compounds of the invention as an active therapeutic substance, in particular in the treatment of diseases mediated by RORγ. In another embodiment, the invention relates to the use of compounds of the invention in the preparation of a medicament for the treatment of diseases mediated by RORγ.

Examples of such diseases include autoimmune or inflammatory diseases such as multiple sclerosis, rheumatoid arthritis, psoriasis, Crohn's disease, inflammatory bowel disease, Sjorgen's syndrome, optic neuritis, chronic obstructive pulmonary disease, asthma, type I diabetes, neuromyelitis optica, Myasthenia Gravis, uveitis, Guillain-Barre syndrome, psoriatic arthritis, Graves' disease, allergic contact dermatitis, systemic lupus erythematosus, cutaneous lupus erythematosus, ankylosing spondylitis. Hashimoto Thyroiditis, Dry Eye, glomerulonephritis, myocarditis and cancer diseases including multiple myeloma and lytic bone disease associated with multiple myeloma, acute myelogenous leukemia (AML), head and neck squamous cell carcinoma, bladder carcinoma, gastric cancer, hepatocellular carcinoma, melanoma, medulloblastoma and colon cancer.

The invention includes the use of compounds of the invention for the preparation of a composition for treating or ameliorating diseases mediated by RORγ in a subject in need thereof, wherein the composition comprises a mixture of one or more of the compounds of the invention and an optional pharmaceutically acceptable excipient.

The compounds of the invention may be used alone or in combination with one or more other therapeutic agents. Accordingly the present invention provides a combination comprising a compound of Formula (I) or a pharmaceutically acceptable salt thereof and one or more other therapeutic agents. Such combinations may be presented individually (wherein each active is in separate composition) or the actives are presented in a combined composition.

This invention provides a combination of a compound of Formula (I), or a pharmaceutically acceptable salt thereof, and one or more therapeutic agents for the treatment of an inflammatory disease and/or an autoimmune disease, for example, a TNF-a inhibitor; a non-selective COX-1/COX-2 inhibitor; a selective COX-2 inhibitor, such as celecoxib; agents including methotrexate, leflunomide, sulfasalazine, azathioprine, penicillamine, bucillamine, actarit, mizoribine, lobenzarit, hydroxychloroquine, d-penicillamine, aurothiomalate, auranofin, parenteral and/or oral gold, cyclophosphamide, a BAFF/APRIL inhibitor, CTLA-4-Ig, or a mimetic of CTLA-4-Ig; 5-lipoxygenase (5-LO) inhibitor, or a 5-lipoxygenase activating protein (FLAP) antagonist; a leukotriene modifier, including a leukotriene receptor antagonist, such as montelukast, zafirlukast, pranlukast; a phosphodiesterase type IV (PDE-IV) inhibitor, such as cilomilast (ariflo) or roflumilast; an antihistamine H1 receptor antagonist; anticholinergic agents such as muscarinic antagonists (ipratropium bromide and tiotropium bromide), as well as selective muscarinic M3 antagonists; β-adrenoceptor agonists such as salmeterol, formoterol, arformoterol, terbutaline, metaproterenol, albuterol and the like; a DP receptor antagonist, such as S-5751 and laropiprant; TP receptor antagonists such as seratrodast; neurokinin antagonists (1 NK2); VLA-4 antagonists; a corticosteroid, such as triamcinolone acetonide, budesonide, beclomethasone, fluticasone and mometasone; insulin-like growth factor type I (IGF-1) mimetic; kinase inhibitors including Janus Kinase inhibitors (e.g., JAK 1 and/or JAK2 and/or JAK 3 and/or TYK2), p38 MAPK, Syk or IKK2; rituximab; selective co-stimulation modulator such as abatacept; IL-1 inhibitor anakinra, IL-6 inhibitor tocilizumab, and IL 12/IL-23 inhibitor ustekimumab; anti-IL 17 antibody, anti-IL 17R antibody, anti-IL21 antibody, or anti-IL22 antibody, S1P1 agonists including fingolimod; interferon beta 1; natalizumab; a mTOR inhibitor such as rapamycin, cyclosporine, tacrolimus; non-steroidal antiinflammatory agent (NSAID), including alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen, indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac, flufenamic acid, meclofenamic acid, mefenamic acid, niflumic acid, tolfenamic acid, diflunisal and flufenisal, isoxicam, piroxicam, sudoxicam, tenoxican, acetyl salicylic acid, apazone, bezpiperylon, feprazone, mofebutazone, oxyphenbutazone, phenylbutazone; fumaric acid derivative, BG-12; chemokine or chemokine receptor inhibitor, such as a CCR-1, CCR-2, CCR-3 and CCR-9 antagonist.

This invention further provides a combination of a compound of Formula (I), or a pharmaceutically acceptable salt thereof, and one or more therapeutic agents for the treatment of multiple myeloma, for example. Bortezomib-dexamethasone. Bortezomib-dexamethasone-cyclophosphamide, Bortezomib-dexamethasone-lenalidomide, Lenalidomide-dexamethasone, Melphalan-prednisone-thalidomide. Melphalan-prednisone-bortezomib, Melphalan-prednisone-lenalidomide, Lenalidomide-dexamethasone-clarithromycin and any of the above combinations plus agents used to treat bone disease in multiple myeloma including bisphosphonates, RANK-L inhibitors such as Denusomab and anabolic bone building drugs such as parathyroid hormone (PTH).

This invention also provides a combination of a compound of Formula (I), or a pharmaceutically acceptable salt thereof, and one or more therapeutic agents for the treatment of colon and/or rectal cancer, for example FOLFOX® (leucovorin [folinic acid], 5-Fluoruracil, and oxaliplatin), FOLFIRI® (leucovorin, 5-Fluoruracil, and irinotecan), CapeOX® (capecitabine and oxaliplatin), any of the above combinations plus either bevacizumab or cetuximab (but not both), 5-Fluoruracil and leucovorin, with or without bevacizumab, Capecitabine, with or without bevacizumab, FOLFOXIRI® (leucovorin, 5-Fluoruracil, oxaliplatin, and irinotecan), Irinotecan, with or without cetuximab, Cetuximab alone, and Panitumumab alone.

### Compositions

The compounds of the invention will normally, but not necessarily, be formulated into pharmaceutical compositions prior to administration to a patient. Accordingly, in another aspect the invention is directed to pharmaceutical compositions comprising a compound of the invention and one or more pharmaceutically acceptable excipient(s).

The pharmaceutical compositions of the invention may be prepared and packaged in bulk form wherein an effective amount of a compound of the invention can be extracted and then given to the patient such as with powders, syrups, and solutions for injection. Alternatively, the pharmaceutical compositions of the invention may be prepared and packaged in unit dosage form. For oral application, for example, one or more tablets or capsules may be administered. A dose of the pharmaceutical composition contains at least a therapeutically effective amount of a compound of this invention (i.e., a compound of Formula I or a salt, particularly a pharmaceutically acceptable salt, thereof). When prepared in unit dosage form, the pharmaceutical compositions may contain from 1 mg to 1000 mg of a compound of this invention.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional therapeutically active compounds.

As used herein, “pharmaceutically acceptable excipient” means a pharmaceutically acceptable material, composition, or vehicle involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically acceptable.

The compounds of the invention and the pharmaceutically acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories, (5) inhalation such as dry powders, aerosols, suspensions, and solutions; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of the invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences(Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

### Compound Preparation

The compounds of Formula (I) may be obtained by using synthetic procedures illustrated in the Schemes below or by drawing on the knowledge of a skilled organic chemist. The reaction sequences provided in these Schemes are applicable for producing compounds of the invention having a variety of different X¹-X⁵, R, R¹, R³, R^{3a}, R⁴, R^{4a}, K¹-K⁴, and A¹-A⁵ groups, as defined above, employing appropriate precursors. The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

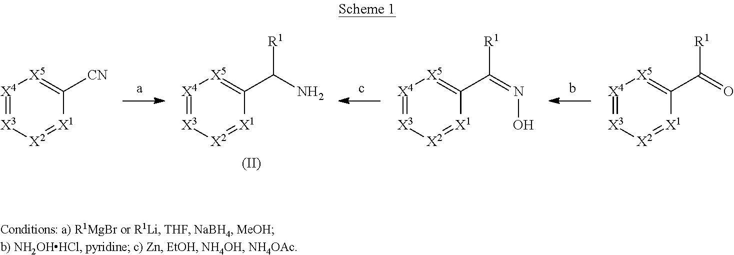

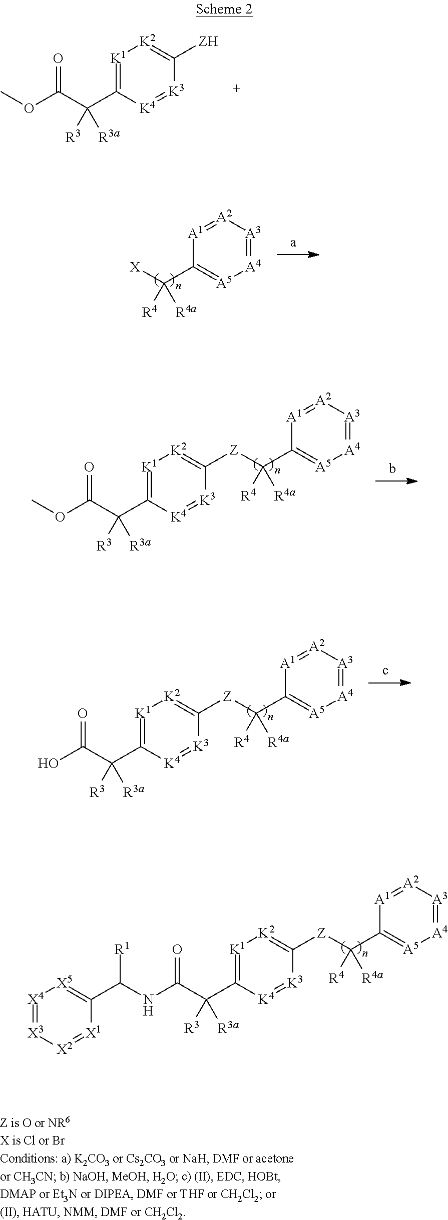

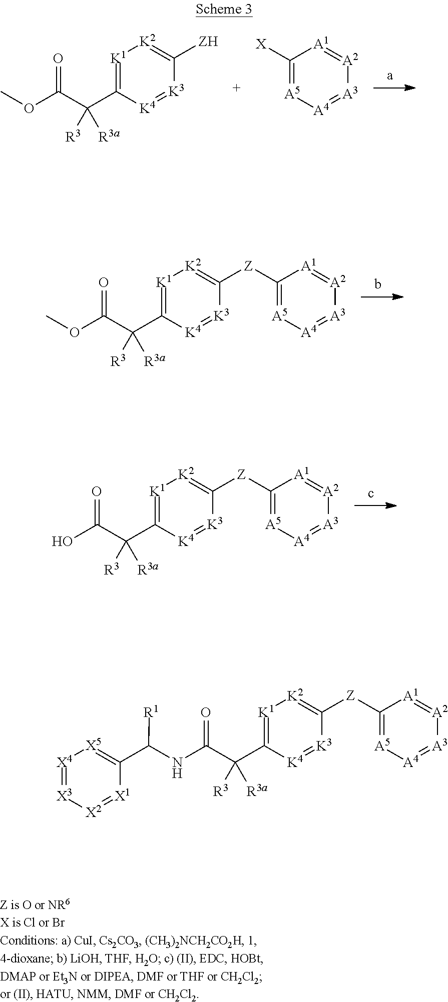

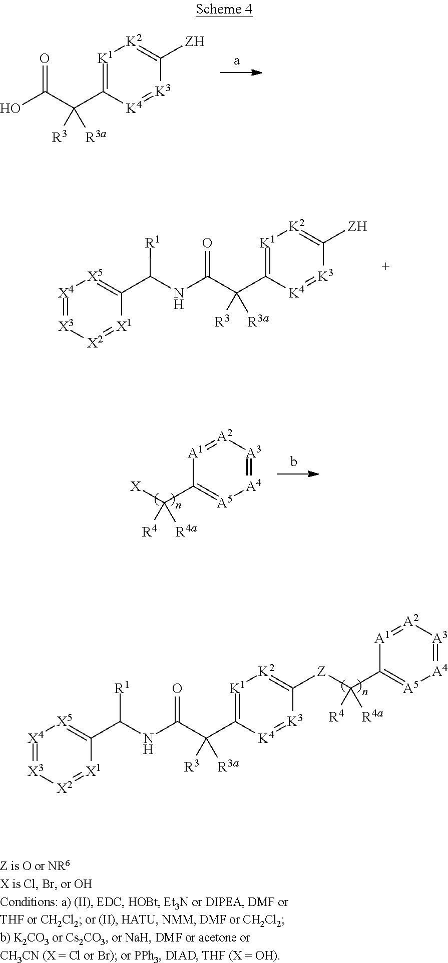

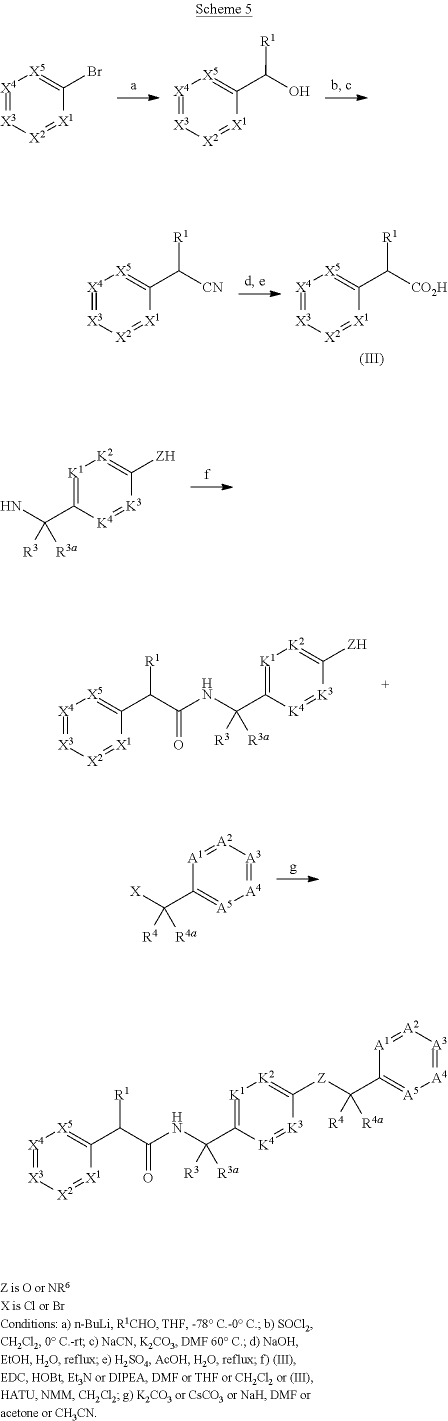

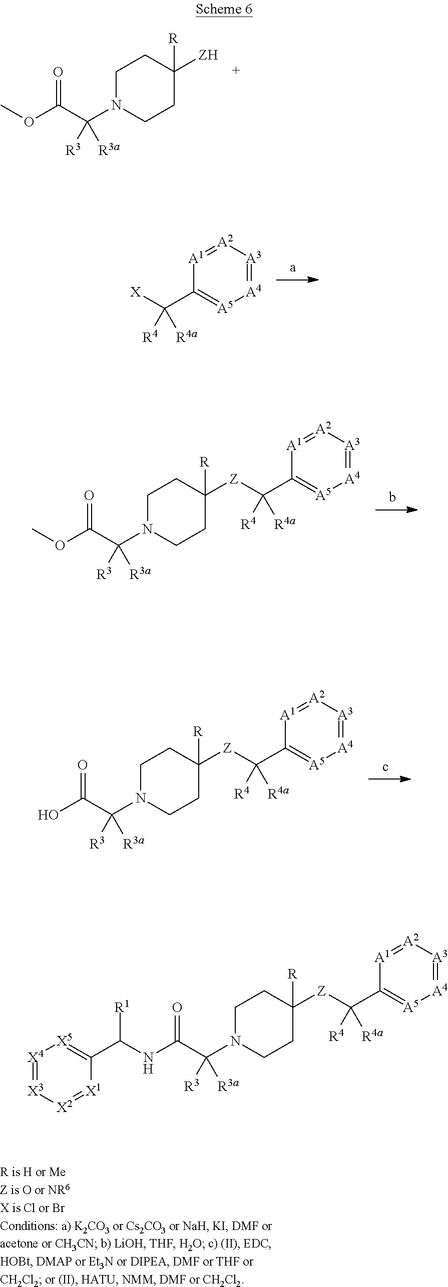

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

Compounds names were generated using the software program ChemBioDraw Ultra V12.0 available from CambridgeSoft Corporation, 100 CambridgePark Drive, Cambridge, Mass. 02140 USA (http://www.cambridgesoft.com).

### ABBREVIATIONS

AcOH acetic acid
AIBN azobisisobutyronitrile
AlCl₃ aluminum trichloride
aq. aqueous
Ar argon gas
Br₂ bromine
CBr₄ carbon tetrabromide
CCl₄ carbon tetrachloride
CH₂Cl₂ dichloromethane
CH₃CN acetonitrile
CH₃I methyl iodide
(CH₂O)ₙ paraformaldehyde
CH₃SO₃H methanesulfonic acid
conc. concentrated
Cs₂CO₃ cesium carbonate
CuBr copper(I) bromide
CuCN copper(I) cyanide
CuI copper(I) iodide
(COCl)₂ oxalyl chloride

### DIPEA N,N-diisopropylethylamine

DMAP 4-(dimethylamino)pyridine
DME 1,2-dimethoxyethane

### DMF N,N-dimethylformamide

DMSO dimethylsulfoxide
EtOAc ethyl acetate
EDC N-(3-dimethylaminopropyl)-N′-ethylcarbodiimide hydrochloride
Et₃N triethylamine
Et₂O diethyl ether
EtOH ethanol
FeSO₄ iron(II) sulfate
h hour(s)
H₂ hydrogen gas
HATU O-(7-azabenzotriazol-1-yl)-N,N,N′,N′-tetramethyluronium hexafluorophosphate
HBr hydrobromic acid
HCl hydrochloric acid
H₂O water
HNO₃ nitric acid
HOBt hydroxybenzotriazole
HPLC high-performance liquid chromatography
H₂SO₄ sulfuric acid
I₂ iodine
i-PrMgCl isopropylmagnesium chloride
K₂CO₃ potassium carbonate
K₃Fe(CN)₆ potassium ferricyanide
KOt-Bu potassium tert-butoxide
K₃PO₄ potassium phosphate tribasic
LCMS liquid chromatography mass spectrometry
LiAlH₄ lithium aluminum hydride
LiOH lithium hydroxide
m-CPBA meta-chloroperbenzoic acid
MeMgBr methyl magnesium bromide
MeOH methanol
Mg magnesium
MgCl₂ magnesium chloride
min minute(s)
MnO₂ manganese dioxide
N₂ nitrogen gas
NaBH₄ sodium borohydride
NaCN sodium cyanide
Na₂CO₃ sodium carbonate
NaH sodium hydride
NaHCO₃ sodium bicarbonate
NaHSO₃ sodium bisulfite
NaN₃ sodium azide
NaOH sodium hydroxide
Na₃SO₄ sodium sulfate
n-BuLi n-butyllithium
NH₄Cl ammonium chloride

### NMM N-methylmorpholine

PCC pyridinium chlorochromate
Pd/C palladium on carbon
Pd(dppf)Cl_{2 [}1,1′-bis(diphenylphosphino)ferrocene]dichloropalladium(II)
Pd(PPh₃)₄ tetrakis(triphenylphosphine)palladium(0)
PhNO₂ nitrobenzene
POCl₃ phosphoryl chloride
PPh₃ triphenylphosphine
p-TsOH para-toluene sulfonic acid
R_{f} retention factor
rt room temperature
Rₜ retention time
SOCl₂ thionyl chloride
TFA trifluoroacetic acid
THF tetrahydrofuran
TLC thin layer chromatography
®T3P 2,4,6-tripropyl-1,3,5,2,4,6-trioxatriphosphorinane 2,4,6-trioxide
Zn zinc powder

### LCMS Conditions

LCMS-P1: Column: Waters Sunfire C18, 3.5 μm, 50×4.6 mm; Temperature: 50° C.; Mobile Phase: A: water (0.05% TFA) B: acetonitrile (0.05% TFA); Gradient: 5% B for 0.2 min, increase to 95% B within 1.2 min, 95% B for 1.6 min, return to 5% B within 0.01 min.; Flow Rate: 1.8 mL/min; Detection: PDA 190-400 nm

LCMS-G7: Column: XBridge C18, 3.6 μm, 50×4.6 mm; Temperature: 50° C.; Mobile Phase: A: water (0.1% formic acid) B: methanol; Gradient: 10% B for 0.1 min. increase to 95% B within 2.5 min, 95% B for 2.5 min, return to 10% B within 0.1 min, 10% B for 2 min.; Flow Rate: 1.0 mL/min; Detection: PDA 190-400 nm

LCMS-G9: Column: XBridge C18, 3.6 μm, 50×4.6 mm; Temperature: 50° C.; Mobile Phase: A: water (0.1% ammonium acetate) B: methanol; Gradient: 10% B for 0.2 min, increase to 95% B within 5 min, 95% B for 2 min, return to 10% B within 0.1 min, 10% B for 2 min.; Flow Rate: 0.8 mL/min; Detection: PDA 190-400 nm

LCMS-G12: Column: Sunfire C18.5 μm, 50×4.6 mm; Temperature: 50° C.; Mobile Phase: A: water (0.1% formic acid) B: methanol; Gradient: 30% B for 0.1 min. increase to 90% B within 4 min, 99% B for 4 min, return to 30% B within 0.1 min, 10% B for 2 min.; Flow Rate: 0.8 mL/min; Detection: PDA 190-400 nm

LCMS-G30: Column: Eclipse XDB C18.5 μm, 250×4.6 mm; Temperature: 50° C.; Mobile Phase: A: water (0.05% TFA) B: acetonitrile (0.05% TFA); Gradient: 30% B for 0.2 min, increase to 95% B within 15 min, 95% B for 5 min, return to 30% B within 3 min 30% B for 5 min.; Flow Rate: 0.8 mL/min; Detection: PDA 190-400 nm

LCMS-X: Column: Eclipse XDB C18.5 μm, 150×4.6 mm; Temperature: 50° C.; Mobile Phase: A: water (0.1% formic acid) B: acetonitrile (0.1% formic acid); Gradient: 10% B for 0.1 min, increase to 90% B within 5 min, 100% within 2 min. 100% B for 4 min, return to 10% B within 0.01 min, 10% B for 1 min.; Flow Rate: 1.0 mL/min; Detection: PDA 190-400 nm

LCMS-T1: Column: Eclipse XDB C18.5 μm, 150×4.6 mm; Temperature: 50° C.; Mobile Phase: water (0.05% TFA) B: acetonitrile (0.05% TFA); Gradient: 5% B for 0.1 min, increase to 95% B within 7 min, 100% within 2 min, return to 5% B within 0.1 min. 5% B for 3 min.; Flow Rate: 1.0 mL/min; Detection: PDA 190-400 nm

### Example 1

### N-((2-chloro-4-methylphenyl)(phenyl)methyl)-2-(4-(pyridin-3-ylmethoxy)phenyl)acetamide

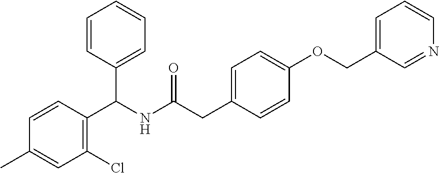 [SMILES: Cc1ccc(C(NC(=O)Cc2ccc(OCc3cccnc3)cc2)c2ccccc2)c(Cl)c1]

### (a) (2-chloro-4-methylphenyl)phenylmethanamine

To a solution of 2-chloro-4-methylbenzonitrile (0.50 g, 3.31 mmol) in anhydrous THF (7 mL) at 0° C. was added 1 M solution of phenyl magnesium bromide in THF (4.96 mL, 4.96 mmol) over 10 minutes and the resulting mixture was warmed to rt. The reaction mixture was stirred at rt for 30 minutes followed by heating to 60° C. and stirred at the same temperature for 2 h. After completion of the imine formation, the reaction mixture was cooled to 0° C. and methanol (10 mL) was added very slowly followed by sodium borohydride (0.188 g, 4.96 mmol). The resulting mixture was warmed to rt and stirred overnight. After completion of the reaction, solvent was removed under vacuum and water (20 mL) was added into the reaction mixture and extracted with ethyl acetate (2×35 mL). The combined organic layers were dried over Na₂SO₄ and concentrated. The product was purified by silica gel column chromatography (10% ethyl acetate/Hexanes) to provide title compound (0.16 g, 21%). ¹H NMR (400 MHz, DMSO-d₆) δ ppm 7.59-7.61 (d, 1H), 7.24-7.32 (m, 4H), 7.15-7.19 (q, 3H), 5.37 (s, 1H), 2.31-2.34 (t, 1H), 2.26 (s, 3H).

### (b) methyl 2-(4-(pyridin-3-ylmethoxy)phenyl)acetate

3-Picolyl chloride hydrochloride (0.2 g, 1.20 mmol) was dissolved in 5N aq. NaOH (5 mL); stirred for 5 minutes and then extracted with diethyl ether. The organic layer was dried over sodium sulfate and concentrated under reduced pressure and used crude in the next step. To a solution of methyl 2-(4-hydroxyphenyl)acetate (0.20 g, 1.20 mmol) in anhydrous acetone (10 mL) at rt was added K₂CO₃ (0.500 g, 3.6 mmol) followed by 3-picolyl chloride and the resulting mixture was heated to reflux overnight. After completion of the reaction, solvent was evaporated under vacuum and water (10 mL) was added to the residue and extracted with ethyl acetate (2×25 mL). The combined organic layers were washed with brine (25 mL), dried over Na₂SO₄, and evaporated to obtain title compound (0.250 g, 80.73%) as a light yellow oil which was used in the next step without further purification. ¹H NMR (400 MHz, DMSO-d₆) δ ppm 8.66 (d, 1H), 8.53-8.55 (dd, 1H), 7.85-7.87 (d, 1H), 7.41-7.44 (dd, 1H), 7.18-7.20 (d, 2H), 6.97-6.99 (d, 2H), 5.14 (s, 2H), 3.60 (s, 5H).

### (c) 2-(4-(pyridin-3-ylmethoxy)phenyl)acetic acid

To a solution of methyl 2-(4-(pyridin-3-ylmethoxy)phenyl)acetate (0.250 g, 0.97 mmol) in methanol (5 mL) at 25° C. was added 2N aqueous solution of NaOH (5 mL) and the resulting mixture was stirred at rt for 1 h. After completion of the reaction, the solvent was evaporated in vacuum and water (10 mL) was added into the residue. The aqueous layer was extracted with ethyl acetate (2×10 mL) to remove the impurities and the organic layers were discarded. The aqueous layer was then made acidic using 5 N aq. HCl and extracted with ethyl acetate (2×25 mL). The organic layer was washed with brine (20 mL) and dried over Na₂SO₄, and evaporated in vacuum to obtain title compound (70 mg, 55%) which was used in the next step.

### (d) N-((2-chloro-4-methylphenyl)(phenyl)methyl)-2-(4-(pyridin-3-ylmethoxy)phenyl)acetamide

To a solution of 2-(4-(pyridin-3-ylmethoxy)phenyl)acetic acid (0.070 g, 0.28 mmol) in DMF (5 mL) at 25° C. was added HOBt (0.054 g, 0.35 mmol), EDC (0.068 g, 0.35 mmol) and DMAP (0.070 g, 0.57 mmol) and the resulting mixture was stirred at rt for 10 minutes. (2-chloro-4-methylphenyl)(phenyl)methanamine (0.074 g, 0.31 mmol) in DMF (1 mL) was added slowly and the stirring at rt was continued overnight. After completion of the reaction, water (10 mL) was added into the reaction mixture slowly in an ice bath and the solid obtained was filtered off and washed with water (10 mL) followed by hexanes (10 mL) and dried under vacuum to obtain title compound (85 mg, 65%). LCMS-X1: 457.6 [M+H]⁺; Rₜ, =5.82 min. ¹H NMR (400 MHz, DMSO-d₆) δ ppm 8.94-8.96 (d, 1H), 8.65-8.66 (d, 1H), 8.53-8.55 (dd, 1H), 7.84-7.86 (d, 1H), 7.40-7.43 (dd, 1H), 7.23-7.33 (m, 5H), 7.13-7.20 (m, 5H), 6.94-6.96 (d, 2H), 6.33-6.35 (d, 1H), 5.12 (s, 2H), 3.45 (s, 2H), 2.28 (s, 3H).

### Example 2

### N-((4-chloro-3-methylphenyl)(phenyl)methyl)-2-(4-((2-methylpyridin-3-yl)methoxy)phenyl)acetamide

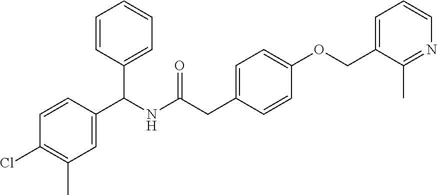 [SMILES: Cc1cc(C(NC(=O)Cc2ccc(OCc3cccnc3C)cc2)c2ccccc2)ccc1Cl]

### (a) (2-methylpyridin-3-yl)methanol

To the suspension of LiAlH₄ (2.62 g, 68.8 mmol) in THF (50 mL) was added the solution of methyl 2-methylnicotinate (5.2 g, 34.4 mmol) in THF (10 mL) slowly at 0° C. After the addition, the resulting mixture was stirred at rt overnight. Water (2.62 mL), 2 M aq. NaOH (5.2 mL), and H₂O (7.86 mL) were added successively. Then the reaction mixture was stirred at rt for 30 min and the solid precipitated was removed by filtration. The filtrate was concentrated under reduced pressure and the title compound was used in the next step without further purification. LCMS-P1: 124, [M+H]⁺.

### (b) 3-(chloromethyl)-2-methylpyridine

To the solution of (2-methylpyridin-3-yl)methanol (2.4 g, 19.5 mmol) in dichloromethane (20 mL) was added SOCl₂ (3.1 g, 24 mmol) dropwise at 0° C. After stirring at rt for 2 h, the solvent was evaporated in vacuum and the crude title compound was used directly in the next step without further purification. LCMS-P1: 142, [M+H]⁺.

### (c) ethyl 2-(4-((2-methylpyridin-3-yl)methoxy)phenyl)acetate

To the solution of 3-(chloromethyl)-2-methylpyridine (2.8 g, 19.5 mmol) and ethyl 2-(4-hydroxyphenyl)acetate (1.8 g, 10 mmol) in MeCN (10 mL) was added K₂CO₃ (3.45 g, 25 mmol), and then the mixture was heated to reflux overnight. The reaction mixture was evaporated in vacuum; water was added, then extracted with EtOAc. The combined organic layers were washed with brine and dried over Na₂SO₄. After removal of solvent, the residue was purified by column chromatography to afford the title compound as a light-yellow solid (1.41 g, 49%) LCMS-P1: 286, [M+H]⁺.

### (d) 2-(4-((2-methylpyridin-3-yl)methoxy)phenyl)acetic acid hydrochloride

To the suspension of ethyl 2-(4-((2-methylpyridin-3-yl)methoxy)phenyl)acetate (1 g, 3.5 mmol) in THF/Water (10 mL, 1:1) was added NaOH (280 mg, 7 mmol). After stirring at rt for 30 min, the reaction mixture was concentrated under vacuum to remove the excess THF. To the residue aq. HCl (2M) was added slowly until the precipitate appeared. Then the precipitate was collected by filtration to give the title compound as a white solid (742 mg, 82%). LCMS-P1: 258 [M+H]⁺.

### (e) (4-chloro-3-methylphenyl)(phenyl)methanamine

This compound was prepared from 4-chloro-3-methylbenzonitrile and phenylmagnesium bromide essentially as example 1 (a) and the crude title compound was used directly for the next step. LCMS-P1: 215.0 [M-NH₂]⁺; Rₜ=1.261 min.

### (f) N-((4-chloro-3-methylphenyl)(phenyl)methyl)-2-(4-((2-methylpyridin-3-yl)methoxy)phenyl)acetamide

To a solution of 2-(4-((2-methylpyridin-3-yl)methoxy)phenyl)acetic acid hydrochloride (50 mg, 0.17 mmol) in dichloromethane (12 mL) was added (4-chloro-3-methylphenyl)(phenyl)methanamine (54 g, 0.2 mmol), EDC (38 mg, 0.20 mmol), HOBt (27 mg, 0.20 mmol), and DIEA (66 mg, 0.51 mmol). The reaction mixture was stirred at rt overnight. The mixture was diluted with water (30 mL) and extracted by dichloromethane (30 mL×3). The combined organic solvents were washed with 1% HCl aqueous solution, and dried over Na₂SO₄. After removal of solvent, the crude compound was purified by column chromatography on silica gel (EtOAc:petroleum ether=1:4) to provide title compound (18 mg, Yield: 20.7%). LCMS-P1: 471 [M+H]⁺; Rₜ=2.24 min. ¹H NMR (400 MHz, MeOH-d₄) δ ppm 8.38 (d, J=1.6 Hz, 1H), 8.37 (d, J=1.6 Hz 1H), 7.27-7.34 (m, 8H), 7.19 (m, 1H), 7.10 (m, 3H), 6.83-6.88 (m, 2H), 6.11 (s, 1H), 5.10 (s, 2H), 3.53 (m, 2H), 2.60 (s, 3H), 2.30 (s, 3H).

### Example 3

### N-((2,4-dimethylphenyl)(pyridin-2-yl)methyl)-2-(4-((2-methylpyridin-3-yl)methoxy)phenyl)acetamide

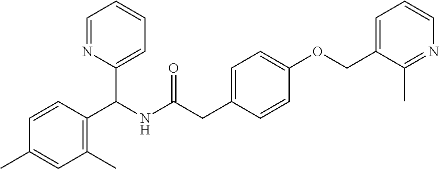 [SMILES: Cc1ccc(C(NC(=O)Cc2ccc(OCc3cccnc3C)cc2)c2ccccn2)c(C)c1]

To the solution of (2,4-dimethylphenyl)(pyridin-2-yl)methanamine hydrochloride (60 mg, 0.23 mmol) in DMF (3 mL) was added 2-(4-((2-methylpyridin-3-yl)methoxy)phenyl)acetic acid (75 mg, 0.29 mmol), EDC (56 mg, 0.29 mmol), HOBt (39 mg, 0.29 mmol), then DIPEA (68 mg, 0.53 mmol). The resulting mixture was heated at 45° C. overnight, the mixture was poured into water, extracted with EtOAc (5 mL×2), and the combined organic layer were washed with brine, dried with Na₂SO₄, and evaporated in vacuum. The residue was purified by preparatory HPLC using 10-100% water/acetonitrile with 0.1% TFA to afford the title compound as a white solid (7 mg, 6%). MS: 452 [M+H]⁺. ¹H NMR (400 MHz, DMSO-d₆) δ ppm 8.82 (d, J=8.4 Hz, 1H), 8.49 (d, J=4.0 Hz, 1H), 8.39 (dd, J=1.6 Hz, 4.8 Hz, 1H), 7.77-7.74 (m, 2H), 7.31-7.18 (m, 5H), 6.96-6.90 (m, 5H), 6.21 (d, J=8.0 Hz, 1H), 5.08 (s, 2H), 3.47 (s, 2H), 2.50 (s, 3H), 2.23 (s, 3H), 2.21 (s, 3H).

### Example 4

### N-((4-chloro-2-methylphenyl)(phenyl)methyl)-2-(4-((2,4-dimethylpyridin-3-yl)methoxy)phenyl)acetamide

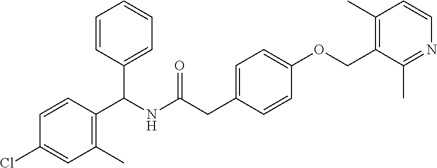 [SMILES: Cc1cc(Cl)ccc1C(NC(=O)Cc1ccc(OCc2c(C)ccnc2C)cc1)c1ccccc1]

### (a) (2,4-dimethylpyridin-3-yl)methanol

To a solution of ethyl 2,4-dimethylnicotinate (1.0 g, 5.57 mmol) in dry tetrahydrofuran (10 mL), lithium aluminum hydride (1.0 M solution in THF, 8.36 mL, 8.36 mmol) was added at 0° C. during 30 minutes and stirred at rt for 1.5 h. After 1.5 h, excess of LiAlH₄ was decomposed with crushed ice and the residue was extracted with ethyl acetate (300 mL). The combined organic layers were dried over Na₂SO₄ and concentrated and the product was purified by silica gel column chromatography (2.5% MeOH: dichloromethane) to provide the title compound (0.650 g, 84.9%). ¹H NMR (400 MHz, DMSO-d₆) δ ppm 8.16-8.18 (d, 1H), 7.01-7.02 (d, 1H), 4.89-4.92, (t, 1H), 4.50-4.51 (d, 2H), 2.50 (s, 3H), 2.34 (s, 3H).

### (b) 3-(chloromethyl)-2,4-dimethylpyridine

To a suspension of (2,4-dimethylpyridin-3-yl)methanol (0.300 g, 2.18 mmol) in dichloromethane (2.0 mL) was added thionyl chloride (0.4 mL, 5.48 mmol) at 0° C. and the reaction was stirred at 0° C. for 30 min. After completion of the reaction, the reaction mixture was concentrated under reduced pressure to provide the title compound (0.340 g, 88.23%). The crude title compound was used in the next step without further purification.

### (c) methyl 2-(4-((2,4-dimethylpyridin-3-yl)methoxy)phenyl)acetate

To a solution of methyl 2-(4-hydroxyphenyl)acetate (0.300 g, 1.80 mmol) in DMF (2.0 mL) was added sodium hydride (60% dispersion in mineral oil, 0.086 g, 2.16 mmol) at 0° C. and the reaction mixture was stirred for 20 min. 3-(chloromethyl)-2,4-dimethylpyridine (0.282 g, 1.80 mmol) was added and the reaction mixture was warmed to 55° C. where it was stirred for 4 h. The reaction mixture was then cooled to room temp and stirred overnight. The reaction mixture was poured into crushed ice and extracted with dichloromethane (250 mL). The organic layer was dried over sodium sulfate and concentrated under reduced pressure to give the crude product. The product was purified by silica gel column chromatography (14% EtOAc/Hexane) to provide the title compound (0.128 g, 25%).

### (d) 2-(4-((2,4-dimethylpyridin-3-yl)methoxy)phenyl)acetic acid

This compound was synthesized from methyl 2-(4-((2,4-dimethylpyridin-3-yl)methoxy)phenyl)acetate essentially as described in example 2 (d). The crude title compound obtained was carried forward to the next step.

### (e) (4-chloro-2-methylphenyl)(phenyl)methanamine

This compound was synthesized from 4-chloro-2-methylbenzonitrile and phenylmagnesium bromide essentially as described in example 1 (a). (0.16 g, 20.83%). ¹H NMR (400 MHz, DMSO-d₆) δ ppm 7.59-7.61 (d, 1H), 7.24-7.32 (m, 4H), 7.15-7.19 (q, 3H), 5.37 (s, 1H), 2.31-2.34 (t, 1H), 2.26 (s, 3H).

### (f) N-((4-chloro-2-methylphenyl)(phenyl)methyl)-2-(4-((2,4-dimethylpyridin-3-yl)methoxy)phenyl)acetamide

This compound was synthesized from 2-(4-((2,4-dimethylpyridin-3-yl)methoxy)phenyl)acetic acid and (4-chloro-2-methylphenyl)(phenyl)methanamine essentially as example 1 (d) and was purified using EtOAc: Hexanes (72%) as the mobile phase to provide the title compound (0.025 g, 13.96%). LCMS-X1: 485.5 [M+H]⁺; Rₜ=5.53 min. ¹H NMR (400 MHz, DMSO-d₆) δ ppm 8.36-8.38 (d, 1H), 6.99-7.31 (m, 13H), 6.48-6.50 (d, 1H), 6.22-6.24 (d, 1H), 5.02 (s, 2H), 3.64 (s, 2H), 2.62 (s, 3H), 2.36 (s, 3H), 2.33 (s, 3H).

### Example 5

### N-((2-chloro-4-methylphenyl)(phenyl)methyl)-2-(4-(pyrimidin-5-ylmethoxy)phenyl)acetamide

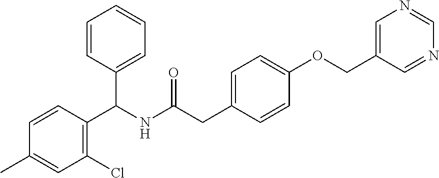 [SMILES: Cc1ccc(C(NC(=O)Cc2ccc(OCc3cncnc3)cc2)c2ccccc2)c(Cl)c1]

### (a) Pyrimidin-5-ylmethanol

To a solution of pyrimidine-5-carbaldehyde (0.300 g, 2.77 mmol) in methanol (6 mL), sodium borohydride (0.158 g, 4.16 mmol) was added at 0° C. over 10 minutes and the reaction mixture was stirred at the same temperature for 30 min. After completion of the reaction, the solvent was removed under vacuum. The residue obtained was poured into water (30 mL), extracted with ethyl acetate (150 mL). The organic layer was dried over sodium sulfate and concentrated to provide the title compound (0.160 g, 52.4%), ¹H NMR (400 MHz, DMSO-d₆) δ ppm 9.18-9.20 (s, 1H), 8.74 (s, 2H), 5.49-5.67 (b, 1H), 4.60 (s, 2H).

### (b) 5-(chloromethyl)pyrimidine

This compound was synthesized from pyrimidin-5-ylmethanol essentially as described in example 2 (b). The crude title compound was obtained and carried forward in the next step.

### (c) methyl 2-(4-(pyrimidin-5-ylmethoxy)phenyl)acetate

This compound was synthesized from methyl 2-(4-hydroxyphenyl)acetate and 5-(chloromethyl)pyrimidine essentially as described in example 4 (c). (0.150 g, 41.55%). ¹H NMR (400 MHz, DMSO-d₆) δ ppm 9.18 (s, 1H), 8.91 (s, 2H), 7.19-7.21 (d, 2H), 6.99-7.01 (d, 2H), 5.17 (s, 2H), 3.61 (s, 2H), 3.60 (s, 3H).

### (d) 2-(4-(pyrimidin-5-ylmethoxy)phenyl)acetic acid hydrochloride

To a solution of methyl 2-(4-(pyrimidin-5-ylmethoxy)phenyl)acetate (0.145 g, 0.56 mmol) in 4 N aq. NaOH (5 mL) was stirred at rt for 30 min. Then reaction mixture was acidified with HCl (6N) during which white solid precipitates out. The precipitate was washed with water, triturated with hexanes (20 mL), and dried under vacuum to provide the title compound (0.065 g, 47%). ¹H NMR (400 MHz, DMSO-d₆) 8 ppm 9.17 (s, 1H), 8.91 (s, 2H), 7.18-7.20 (d, 2H), 6.98-7.00 (d, 2H), 5.17 (s, 2H), 3.50 (s, 2H).

### (e) N-((2-chloro-4-methylphenyl)(phenyl)methyl)-2-(4-(pyrimidin-5-ylmethoxy)phenyl)acetamide

The compound was synthesized from 2-(4-(pyrimidin-5-ylmethoxy)phenyl)acetic acid hydrochloride and (2-chloro-4-methylphenyl)(phenyl)methanamine essentially as example 1 (d) and purified as follows: the reaction mixture was cooled to 0° C. and water (10 mL) was added during which solid precipitates out. The solid obtained was filtered and washed with water and hexanes (20 mL) and then triturated with diethyl ether (30 mL) to provide the title compound (0.045 g, 40%). LCMS-X1: 458.6 [M+H]⁺; Rₜ=6.40 min. ¹H NMR (400 MHz, DMSO-d₆) δ ppm 9.17 (s, 1H), 8.95-8.97 (d, 1H), 8.90 (s, 2H), 7.13-7.34 (m, 10H), 6.96-6.98 (d, 2H), 6.33-6.35 (d, 1H), 5.16 (s, 2H), 3.46 (s, 2H), 2.28 (s, 3H).

### Example 6

### 2-(4-(benzyloxy)phenyl)-N-((4-chlorophenyl)phenyl)methyl)acetamide

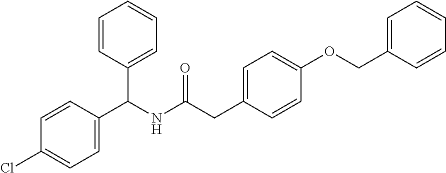 [SMILES: O=C(Cc1ccc(OCc2ccccc2)cc1)NC(c1ccccc1)c1ccc(Cl)cc1]

### (a) N-((4-chlorophenyl)(phenyl)methyl)-2-(4-hydroxyphenyl)acetamide

To a solution of (4-chlorophenyl)(phenyl)methanamine (3.0 g, 11.8 mmol) in DMF (15.0 mL) was added DMAP (2.62 g, 21.4 mmol), EDC (2.72 g, 14.1 mmol), HOBt (2.17 g, 14.1 mmol) followed by 2-(4-hydroxyphenyl)acetic acid (1.63 g, 10.7 mmol) and the reaction mixture was stirred at rt for 1 h. After completion of the reaction, the reaction mixture was concentrated, diluted with EtOAc (50 mL), washed with water (25 mL) and brine (25 mL). After drying (Na₂SO₄) and concentration of the combined organic layers, the product was purified by silica gel column chromatography (40% EtOAc/Hexane) to provide the title compound (4.5 g, 65.2%). MS (ESI+) 352.0 [M+H]. ¹H NMR (400 MHz, DMSO-d₆) δ ppm 9.25 (s, 1H), 8.94-8.96, (d, 1H), 3.37-3.39 (d, 2H), 7.28-7.34 (m, 3H), 7.24-7.26 (m 3H), 7.04-7.06 (d, 2H), 6.65-6.67 (d, 2H), 6.07-6.09 (d, 1H), 4.08 (s, 2H).

### (b) 2-(4-(benzyloxy)phenyl)-N-((4-chlorophenyl)(phenyl)methyl)acetamide

To a solution of N-((4-chlorophenyl)(phenyl)methyl)-2-(4-hydroxyphenyl)acetamide (0.150 g, 0.426 mmol) in DMF (2.0 mL) was added 60%0/sodium hydride (0.020 g, 0.51 mmol) at 0° C. and the reaction was stirred for 20 min. After 20 minutes, benzyl bromide (0.088 g, 0.511 mmol) was added and the reaction was stirred for 1 h. After completion of the reaction, the reaction mixture was dumped into crushed ice, extracted with ethyl acetate (100 mL). After drying (Na₂SO₄) and concentration of the combined organic layers, the product was purified by silica gel column chromatography (14% EtOAc/Hexane) to provide the title compound (0.095 g, 50.53%). LCMS-X1: 442.2 [M+H]⁺; Rₜ=5.37 min. ¹H NMR (400 MHz, CDCl₃) δ ppm 8.99-9.01 (d, 1H), 7.42-7.44 (d, 2H), 7.37-7.40 (m, 4H), 7.30-7.34 (m, 3H), 7.24-7.28 (m, 5H), 7.16-7.19 (d, 2H), 6.91-6.94 (d, 2H), 6.07-6.09 (d, 1H), 5.09 (s, 2H), 3.55 (s, 2H).

### Example 7

### N-(1-(4-chloro-2-methylphenyl)-3-methylbutyl)-2-(4-((2-methylpyridin-3-yl)methoxy)phenyl)acetamide

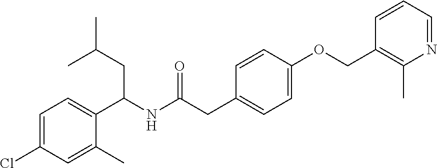 [SMILES: Cc1cc(Cl)ccc1C(CC(C)C)NC(=O)Cc1ccc(OCc2cccnc2C)cc1]

### (a) 1-(4-chloro-2-methylphenyl)-3-methylbutan-1-amine

To a solution of 4-chloro-2-methyl benzonitrile (0.5 g, 3.29 mmol) in dry THF (5 mL) at rt was added isobutylmagnesium bromide (1.2 M solution in Et₂O, 6.8 mL, 8.24 mmol) dropwise and the reaction mixture was heated to 70° C. for 5 h. After completion of the imine formation the reaction mixture was quenched with saturated aq. NH₄Cl solution and the product was extracted with EtOAc. The organic layer was dried over anhydrous sodium sulfate and the solvent was removed under vacuum. The crude product was dissolved in dry MeOH (10 mL) cooled to 0° C. and sodium borohydride was added (146 mg, 3.95 mmol). The reaction mixture was stirred at rt for 1 hour. The reaction mixture was quenched with brine and the solvent was removed under reduced pressure. The product was extracted with EtOAc. The organic layer was dried over anhydrous sodium sulfate and the solvent was removed under vacuum. The crude product was purified by flash column chromatography (neutral alumina, eluent 2-5% MeOH in CH₂Cl₂) to afford the title compound (130 mg, 17%) as a yellow viscous liquid.

### (b) N-(1-(4-chloro-2-methylphenyl)-3-methylbutyl)-2-(4-hydroxyphenyl)acetamide

To a stirred solution of 4-hydroxyphenyl acetic acid (100 mg, 0.66 mmol) in dry DMF (5 mL) were added HATU (299 mg, 0.78 mmol) followed by 1-(4-chloro-2-methylphenyl)-3-methylbutan-1-amine (130 mg, 10.66 mmol) in dry DMF (2 mL) and NMM (0.2 mL, 1.97 mmol) at 0° C. The reaction mixture was slowly warmed to rt and stirred for a further 4 h. The reaction mixture was diluted with EtOAc. The organic layer was washed with water and brine solution. The organic layer was dried over anhydrous sodium sulfate and the solvent was removed under vacuum. The crude product was purified by column chromatography (silica 60-120 mesh, eluent 30-40% EtOAc in petroleum ether) to afford the title compound (100 mg, 44%). ¹H NMR (400 MHz, MeOH-d₄) δ ppm 7.21-7.18 (m, 1H), 7.14 (m, 2H), 7.08-7.06 (d, J=8.5 Hz, 2H), 6.72-6.69 (d, J=8.5 Hz, 2H), 5.17-5.11 (m, 1H), 3.41-3.40 (d, J=4.3 Hz, 2H), 2.35 (s, 3H), 1.69-1.61 (m, 3H), 0.94-0.93 (m, 6H)

### (c) N-(1-(4-chloro-2-methylphenyl)-3-methylbutyl)-2-(4-((2-methylpyridin-3-yl)methoxy)phenyl)acetamide

To a stirred solution of N-(1-(4-chloro-2-methylphenyl)-3-methylbutyl)-2-(4-hydroxyphenyl)acetamide (100 mg, 0.29 mmol) in dry MeCN (10 mL) was added Cs₂CO₃ (188 mg, 0.57 mmol) followed by 3-(chloromethyl)-2-methylpyridine (60 mg, 0.43 mmol) in a sealed tube and the reaction mixture was heated at 80° C. for 16 h. The reaction mixture was diluted with EtOAc. The organic layer was washed with water and brine. The organic layer was dried over anhydrous sodium sulfate and the solvent was removed under vacuum. The crude product was purified by column chromatography (silica 60-120 mesh, eluent 20% EtOAc in CH₂Cl₂) followed by preparative TLC on silica gel (eluent 20% EtOAc in CH₂Cl₂) to afford the title compound (35 mg, 27%). LCMS-G30: 452.3 [M+H]⁺; Rₜ=16.07 min. ¹H NMR (400 MHz, CDCl₃) δ ppm 8.50-8.48 (dd, J=4.9 Hz, 1.6 Hz, 1H), 7.75-7.73 (dd, J=7.7 Hz, 1.4 Hz, 1H), 7.20-7.17 (m, 3H), 7.13-7.09 (m, 2H), 6.99-6.96 (d, J=8.5 Hz, 2H), 6.93-6.91 (d, J=8.3 Hz, 1H), 5.52-5.50 (d, J=8.0 Hz, 1H), 5.21-5.15 (m, 1H), 5.06 (s, 2H), 3.51-3.50 (d, J=4.3 Hz, 2H), 2.61 (s, 3H), 2.37 (s, 3H), 1.51-1.42 (m, 3H), 0.93-0.88 (m, 6H).

### Example 8

### N-((2,4-dimethylphenyl)(phenyl)methyl)-2-(4-(2-(2-methylpyridin-3-yl)ethoxy)phenyl)acetamide

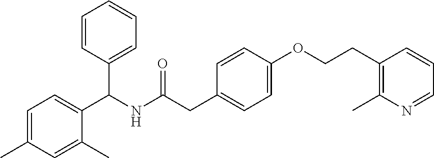 [SMILES: Cc1ccc(C(NC(=O)Cc2ccc(OCCc3cccnc3C)cc2)c2ccccc2)c(C)c1]

### (a) 1-(2-methylpyridin-3-yl)ethanone

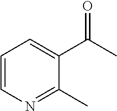

To a solution of freshly distilled acetyl acetone (40.0 g, 0.399 mol) in dry toluene (400 mL) were added freshly distilled acrolein (35.6 g, 0.599 mol) followed by ammonium acetate (52.0 g, 0.799 mol). The reaction mixture was stirred at 120° C. for 16 h. The reaction mixture was filtered through a celite bed and washed with MeOH. The solvent was evaporated under reduced pressure and the crude product was purified by column chromatography (silica gel 60-120 mesh, eluent 50-70% EtOAc in petroleum ether) to afford the title compound (8.5 g, 16%). ¹H NMR (300 MHz, CDCl₃) δ ppm 8.62-8.60 (d, J=4.8 Hz, 1H), 7.99-7.96 (d, J=7.9 Hz, 1H), 7.25-7.23 (m, 1H), 2.76 (s, 3H), 2.61 (d, J=1.1 Hz, 3H)

### (b) 2-(2-methylpyridin-3-yl)-1-morpholinoethanethione

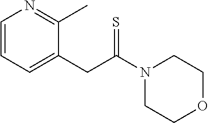 [SMILES: Cc1ncccc1CC(=S)N1CCOCC1]

A mixture of 1-(2-methylpyridin-3-yl)ethanone (8.5 g, 0.062 mol), morpholine (5.4 g, 0.062 mol) and sulfur (2.0 g, 0.062 mol) were added and the reaction mixture was refluxed at 120° C. for 8 h. The reaction mixture was poured into ice-water and the product was extracted with CH₂Cl₂. The organic layer was washed with brine, dried over anhydrous sodium sulfate and the solvent was removed under vacuum. The crude product was purified by flash column chromatography (silica gel 230-400 mesh, eluent 70-80% EtOAc in petroleum ether) to afford the title compound (3.8 g, 26%) as brown solid. ¹H NMR (300 MHz, CDCl₃) δ ppm 8.42-8.40 (d, J=4.8 Hz, 1H), 7.51-7.48 (d, J=7.5 Hz, 1H), 7.15-7.11 (dd, J=7.7 Hz, 4.8 Hz, 1H), 4.41-4.38 (m, 2H), 4.21 (s, 2H), 3.82-3.79 (m, 2H), 3.53 (m, 4H), 2.50 (s, 3H)

### (c) methyl 2-(2-methylpyridin-3-yl)acetate

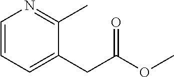

To a solution of 2-(2-methylpyridin-3-yl)-1-morpholinoethanethione (1.5 g, 6.35 mmol) in MeOH (3 mL) was added conc.H₂SO₄ (3 mL) at 0° C. The reaction mixture was allowed to warm to rt and then heated to 100° C. for 2 h. The solvent was removed under reduced pressure and the residue was dissolved in water. The pH of the aqueous layer was adjusted to 8 using saturated Na₂CO₃ solution. The product was extracted with CH₂Cl₂. The organic layer was washed with brine and dried over anhydrous sodium sulfate. Solvent was removed under reduced pressure and the crude product was purified by column chromatography (silica gel 60-120 mesh, eluent 60%0/EtOAc in petroleum ether) to afford the title compound (400 mg, 38%). ¹H NMR (300 MHz, CDCl₃) δ ppm 8.43-8.41 (dd, J=4.8 Hz, 1.5 Hz, 1H), 7.51-7.48 (m, 1H), 7.13-7.09 (dd, J=7.7 Hz, 4.8 Hz, 1H), 3.71 (s, 3H), 3.65 (s, 2H), 2.55 (s, 3H)

### (d) 2-(2-methylpyridin-3-yl)ethanol

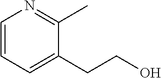

To a solution of methyl 2-(2-methylpyridin-3-yl)acetate (400 mg, 2.4 mmol) in dry THF (5 mL) was added LiAlH₄ (1.93 mL, 3.87 mmol, 2M in THF) drop wise at 0° C. The reaction mixture was stirred at 0° C. for 1 hour. The reaction mixture was quenched carefully with water. The reaction mixture was diluted with EtOAc and filtered through a celite bed. The organic layer was dried over anhydrous Na₂SO₄ and concentrated under vacuum to give the title compound (250 mg, 75%) which was used as such for the next step. ¹H NMR (400 MHz, CDCl₃) δ ppm 8.37-8.36 (dd, J=4.7 Hz, 1.2 Hz, 1H), 7.50-7.48 (d, J=7.7 Hz, 1H), 7.11-7.08 (dd, J=7.5 Hz, 4.8 Hz, 1H), 3.90-3.87 (t, J=6.8 Hz, 2H), 2.92-2.89 (t, J=6.7 Hz, 2H), 2.57 (s, 3H)

### (e) N-((2,4-dimethylphenyl)(phenyl)methyl)-2-(4-hydroxyphenyl)acetamide

To a stirred solution of 4-hydroxyphenyl acetic acid (100 mg, 0.66 mmol) in dry DMF (5 mL) were added HATU (300 mg, 0.78 mmol) followed by (2,4-dimethylphenyl)(phenyl)methanamine (138 mg, 0.66 mmol) and NMM (0.3 mL, 2.63 mmol) at 0° C. The reaction mixture was slowly warmed to rt and stirred for 3 h. The reaction mixture was diluted with EtOAc. The organic layer was washed with 10% NaHCO₃ solution, water, and brine, successively. The organic layer was dried over anhydrous sodium sulfate and the solvent was removed under vacuum. The crude product was purified by washing with diethyl ether to give the title compound (205 mg, 91%) as off-white solid. ¹H NMR (400 MHz, CDCl₃) δ ppm 9.20 (s, 1H), 8.78-8.76 (d, J=8.3 Hz, 1H), 7.32-7.28 (m, 2H), 7.25-7.23 (m, 1H), 7.16-7.14 (m, 2H), 7.05-7.03 (d, J=8.5 Hz, 1H), 6.96-6.95 (m, 3H), 6.66-6.64 (d, J=8.5 Hz, 2H), 6.18-6.16 (d, J=8.5 Hz, 1H), 3.33 (s, 2H), 2.23 (s, 3H), 2.14 (s, 3H)

### (f) N-((2,4-dimethylphenyl)(phenyl)methyl)-2-(4-(2-(2-methylpyridin-3-yl)ethoxy)phenyl)acetamide

A solution of diisopropyl azodicarboxylate (90 mg, 0.433 mmol) in dry THF (2 mL) was added drop-wise to a solution of N-((2,4-dimethylphenyl)(phenyl)methyl)-2-(4-hydroxyphenyl)acetamide (100 mg, 0.29 mmol), 2-(2-methylpyridin-3-yl)ethanol (40 mg, 0.29 mmol) and triphenylphosphine (114 mg, 0.43 mmol) in dry THF (6 mL) at rt. The reaction mixture was further stirred at rt for 2 h. The reaction mixture was concentrated under reduced pressure and the crude product was purified by flash column chromatography (silica gel 230-400 mesh, eluent 40-50% EtOAc in petroleum ether) followed by preparative HPLC using 10-100% water/acetonitrile with 0.1% TFA to give the title compound (15 mg, 11%). LCMS-G7: 465.5 [M+H]⁺; Rₜ, =6.10 min. ¹H NMR (400 MHz, CDCl₃) δ ppm 8.41-8.40 (d, J=3.8 Hz, 1H), 7.54-7.52 (d, J=7.6 Hz, 1H), 7.26-7.26 (m, 3H), 7.19-7.17 (d, J=8.5 Hz, 2H), 7.12-7.09 (m, 1H), 7.07-7.05 (m, 2H), 6.98 (s, 1H), 6.93-6.91 (m, 1H), 6.87-6.85 (d, J=8.5 Hz, 2H), 6.77-6.76 (d, J=7.6 Hz, 1H), 6.37-6.35 (d, J=8.2 Hz, 1H), 5.91-5.89 (d, J=8.2 Hz, 1H), 4.19-4.16 (t, J=6.7 Hz, 2H), 3.58 (s, 2H), 3.13-3.10 (t, J=6.7 Hz, 2H), 2.62 (s, 3H), 2.29 (s, 3H), 2.21 (s, 3H).

### Example 9

### N-((2,4-dichloro-5-fluorophenyl)(phenyl)methyl)-2-(4-((2-methylpyridin-3-yl)methoxy)phenyl)acetamide

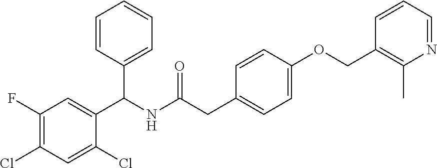 [SMILES: Cc1ncccc1COc1ccc(CC(=O)NC(c2ccccc2)c2cc(F)c(Cl)cc2Cl)cc1]

### (a) (2,4-dichloro-5-fluorophenyl)(phenyl)methanamine

This compound was synthesized from 2,4-dichloro-5-fluoro benzonitrile and phenylmagnesium bromide essentially as described in example 1 (a). ¹H NMR (400 MHz, CDCl₃) δ ppm 7.52-7.49 (d, J=10.0 Hz, 1H), 7.40-7.25 (m, 6H), 5.56 (s, 1H).

### (b) N-((2,4-dichloro-5-fluorophenyl)(phenyl)methyl)-2-(4-hydroxyphenyl)acetamide

This compound was synthesized from (2,4-dichloro-5-fluorophenyl)(phenyl)methanamine and 2-(4-hydroxyphenyl)acetic acid essentially as example 7 (b) and was purified by column chromatography (silica 60-120 mesh, eluent 30-40% EtOAc in petroleum ether) to give the title compound (300 mg, 57%) as a white solid. ¹H NMR (400 MHz, MeOH-d₄) δ ppm 7.58-7.56 (d, J=6.7 Hz, 1H), 7.36-7.27 (m, 3H), 7.18-7.11 (m, 5H), 6.74-6.72 (d, J=8.5 Hz, 2H), 6.38-6.36 (m, 1H), 3.48 (d, J=1.8 Hz, 2H).

### (c) N-((2,4-dichloro-5-fluorophenyl)phenyl)methyl)-2-(4-((2-methylpyridin-3-yl)methoxy)phenyl)acetamide

This compound was synthesized from N-((2,4-dichloro-5-fluorophenyl)(phenyl)methyl)-2-(4-hydroxyphenyl)acetamide and 3-(chloromethyl)-2-methylpyridine essentially as example 7(c) and was purified by column chromatography (silica 60-120 mesh, eluent 5-10% MeOH in CH₂Cl₂) followed by preparative TLC on silica gel (eluent 20% EtOAc in CH₂Cl₂) to afford the title compound (40 mg, 16%). LCMS-G7: 508.0 [M+H]⁺; Rₜ=3.97 min ¹H NMR (400 MHz, MeOH-d₄) δ ppm 8.38-8.37 (dd, J=5.0 Hz, 1.5 Hz, 1H), 7.89-7.87 (dd, J=7.7 Hz, 1.4 Hz, 1H), 7.59-7.57 (d, J=6.8 Hz, 1H), 7.37-7.29 (m, 4H), 7.27-7.25 (d, J=8.8 Hz, 2H), 7.18-7.16 (m, 2H), 7.13-7.11 (d, J=10.0 Hz, 1H), 7.00-6.98 (d, J=8.8 Hz, 2H), 6.38 (s, 1H), 5.13 (s, 2H), 3.55-3.54 (d, J=2.8 Hz, 2H), 2.58 (s, 3H).

### Example 10

### N-((2,4-dimethylphenyl)(phenyl)methyl)-2-(4-(((2-methylpyridin-3-yl)methyl)amino)phenyl)acetamide

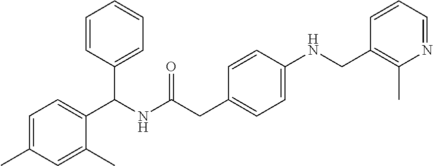 [SMILES: Cc1ccc(C(NC(=O)Cc2ccc(NCc3cccnc3C)cc2)c2ccccc2)c(C)c1]

### (a) N-((2,4-dimethylphenyl)(phenyl)methyl)-2-(4-nitrophenyl)acetamide

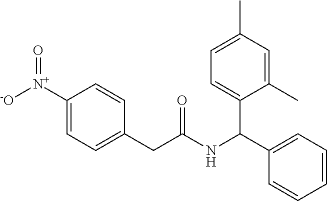

To a stirred solution of 2-(4-nitrophenyl)acetic acid (362 mg, 2 mmol) in 12 mL of dichloromethane were added HOBt (540 mg, 4 mmol), EDC (768 mg, 4 mmol), DIPEA (1 g, 8 mmol) and (2,4-dimethylphenyl)(phenyl)methanamine (506 mg, 2.4 mmol). The resulting mixture was stirred at rt overnight. Dichloromethane (10 mL) was added to the mixture and the mixture was washed with diluted HCl (10 mL×3), brine (10 mL×3), and the organic layer was dried over Na₂SO₄. After removal of solvent, the residue was recrystallized from ethyl acetate to give the title product (600 mg, yield: 80%) as a white solid. LCMS-P1:397 [M+Na]⁺; Rₜ: 1.75 min.

### (b) 2-(4-aminophenyl)-N-((2,4-dimethylphenyl)(phenyl)methyl)acetamide

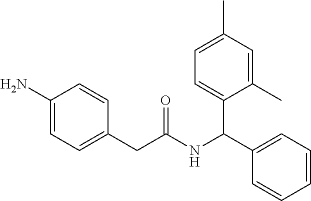 [SMILES: Cc1ccc(C(NC(=O)Cc2ccc(N)cc2)c2ccccc2)c(C)c1]

To a solution of N-((2,4-dimethylphenyl)(phenyl)methyl)-2-(4-nitrophenyl)acetamide (600 mg, 1.6 mmol) in saturated aq. NH₄Cl (15 mL) were added Zn dust (520 mg, 8 mmol) and DMF (10 mL) under nitrogen. The reaction mixture was stirred at 50° C. for 12 h. After completion of the reaction, Zinc was separated by filtration and water (100 mL) was added to the reaction mixture. The mixture was extracted with dichloromethane (3×100 mL). The combined organic layers were dried over Na₂SO₄. Removal of solvent gave the title compound (530 mg, yield: 96%) as a yellow solid. LCMS-P1: 345 [M+H]⁺; Rₜ: 1.43 min.

### (c) N-((2,4-dimethylphenyl)(phenyl)methyl)-2-(4-(((2-methylpyridin-3-yl)methyl)amino)phenyl)acetamide

To a solution of 2-(4-aminophenyl)-N-((2,4-dimethylphenyl)(phenyl)methyl)acetamide (58 mg, 0.169 mmol) in MeCN (10 mL) was added 3-(chloromethyl)-2-methylpyridine (27 mg, 0.152 mmol) and K₂CO₃ (63 mg, 0.457 mmol). The mixture was stirred at 65° C. for 12 h. After completion of the reaction, water (30 mL) was added to the reaction mixture, and the mixture was extracted with ethyl acetate (50 mL×3). The combined organic layers were washed with brine (15 mL×3) and dried over Na₂SO₄. The solvent was evaporated to give a residue which was recrystallized from 10% ethyl acetate in petroleum ether to give the title compound as a white solid (35 mg, 51.3%). LCMS-P1: 450 [M+H]⁺; Rₜ=1.97 min. ¹H NMR (400 MHz, MeOH-d₄) δ ppm 8.28 (d, J=4.8 Hz, 1H), 7.73 (d, J=7.6 Hz, 1H), 7.74-6.55 (m, 12H), 6.55 (d, J=8.8 Hz, 1H), 6.26 (s, 1H), 4.33 (s, 2H), 3.43 (s, 2H), 2.53 (s, 3H), 2.16 (s, 3H), 2.15 (s, 3H).

### Example 11

### N-((4-chloro-2-methylphenyl)(1-methyl-1H-pyrazol-4-yl)methyl)-2-(4-((2-methylpyridin-3-yl)methoxy)phenyl)acetamide

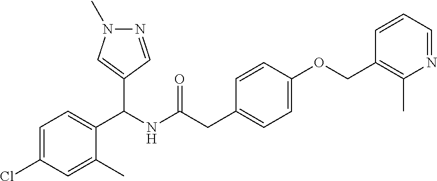 [SMILES: Cc1cc(Cl)ccc1C(NC(=O)Cc1ccc(OCc2cccnc2C)cc1)c1cnn(C)c1]

### (a) ethyl 2-(4-((2-methylpyridin-3-yl)methoxy)phenyl)acetate

This compound was synthesized from ethyl 2-(4-hydroxyphenyl)acetate and 3-(chloromethyl)-2-methylpyridine hydrochloride essentially as described in example 2 (c). (1.41 g, 49%). MS: 286, [M+H]⁻.

### (b) 2-(4-((2-methylpyridin-3-yl)methoxy)phenyl)acetic acid hydrochloride

To the suspension of ethyl 2-(4-((2-methylpyridin-3-yl)methoxy)phenyl)acetate (1 g, 3.5 mmol) in THF/Water (10 mL, 1:1) was added NaOH (280 mg, 7 mmol). After stirring at rt for 30 min, the reaction mixture was evaporated in vacuum to remove the excess THF. HCl (2M) was added slowly to the residue until the precipitate appeared. Then the precipitate was collected to give the title compound as a white solid (742 mg, 82%). MS: 258 [M+H]⁺.

### (c) (4-chloro-2-methylphenyl)(1-methyl-1H-pyrazol-4-yl)methanamine

To a stirred solution of 4-bromo-1-methyl-1H-pyrazole (1 g, 6.2 mmol) in 20 mL of THF was added dropwise n-BuLi (2.72 mL, 6.8 mmol, 2.5 N in hexanes) at −78° C. under nitrogen. After stirring at that temperature for 30 min, 4-chloro-2-methylbenzonitrile (1.026 g, 6.8 mmol) was added. The reaction mixture was slowly warmed to rt. Methanol (10 mL) was added followed by addition of NaBH₄ (472 mg, 12.4 mmol). After stirring at rt overnight, the mixture was extracted with EtOAc (3×30 mL). The combined organic layers were washed with brine (15 mL) and dried over Na₂SO₄. After removal of solvent, the crude product was purified by silica gel column chromatography (CH₂Cl₂/MeOH=30/1) to give the title compound as colorless oil (150 mg, yield: 10%). LCMS-P1: 236.0 [M+H]⁺, rt=1.129 min.

### (d) N-((4-chloro-2-methylphenyl)(1-methyl-1H-pyrazol-4-yl)methyl)-2-(4-((2-methylpyridin-3-yl)methoxy)phenyl)acetamide

This compound was synthesized from (4-chloro-2-methylphenyl)(1-methyl-1H-pyrazol-4-yl)methanamine and 2-(4-((2-methylpyridin-3-yl)methoxy)phenyl)acetic acid hydrochloride essentially as described in example 3. LCMS-P1: 475.0 [M+H]⁺; Rₜ=1.303 min. ¹H NMR (400 MHz, CDCl₃) δ ppm 8.84 (d, J=7.6 Hz, 1H), 8.61 (d, J=5.2 Hz, 1H), 8.21 (d, J=6.8 Hz, 1H), 7.63 (t, J=6.0 Hz, 1H), 7.35-7.18 (m, 7H), 6.99 (d, J=8.8 Hz, 2H), 6.03 (d, J=8.0 Hz, 1H), 5.20 (s, 2H), 3.75 (s, 3H), 2.64 (s, 3H), 2.21 (s, 3H).

### Example 12

### 2-(2,4-dimethylphenyl)-N-(4-((2-methylpyridin-3-yl)methoxy)benzyl)-2-phenylacetamide

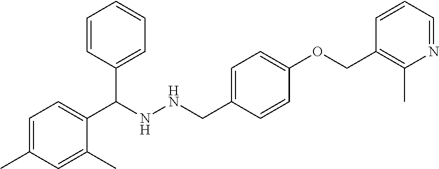 [SMILES: Cc1ccc(C(NNCc2ccc(OCc3cccnc3C)cc2)c2ccccc2)c(C)c1]

### (a) 2-(2,4-dimethylphenyl)-2-phenylacetic acid

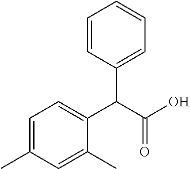 [SMILES: Cc1ccc(C(C(=O)O)c2ccccc2)c(C)c1]

The solution of mandelic acid (10.0 g, 65.7 mmol) in m-xylene (56.79 mL, 460 mmol) was heated to 60-70° C. followed by the addition of SnCl₄ (11.5 mL, 98.6 mmol) over 2 h. The reaction mixture was cooled to rt and then stirred for 6 h at rt. The completion of the reaction was monitored by TLC on silica gel using Hexanes:EtOAc (1:1) as mobile phase. After completion of the reaction, ice-water (100 mL) was added, and the reaction mixture was extracted with diethyl ether (3×250 mL). The combined ether layers were discarded. The remaining reaction mixture was extracted with 8% aqueous Na, CO₃ (10×50 mL) and the combined aqueous layer was then acidified using 6 N HCl (20 mL) and the solid obtained was filtered and dried. The crude solid product was purified using silica gel column chromatography using 20% EtOAc: Hexanes to obtain title compound (6.25 g, 39.63%) as a solid. ¹H NMR (400 MHz, DMSO-d₆) 8 ppm 12.6 (s, 1H), 7.21-7.33 (m, 6H), 7.09-7.12 (m, 1H), 6.97-7.06 (m, 3H), 5.12 (s, 1H), 2.23 (s, 3H), 2.17-2.18 (d, 4H).

### (b) 2-(2,4-dimethylphenyl)-N-(4-hydroxybenzyl)-2-phenylacetamide

To a solution of 2-(2,4-dimethylphenyl)-2-phenylacetic acid (0.25 g, 1.04 mmol) in anhydrous DMF (10 mL) at 25° C. was added EDC (0.237 g, 1.24 mmol) and the reaction was stirred for 30 min at rt under argon atmosphere followed by addition of HOBt (0.19 g, 1.24 mmol), 4-dimethylamino pyridine (0.19 g, 1.56 mmol) and 4-(aminomethyl)phenol (0.154 g, 1.24 mmol), and the reaction mixture was stirred overnight under argon atmosphere. After completion of the reaction, the reaction mixture was cooled and water (10 mL) was added drop wise during which solid precipitated out. The solid precipitate was collected by filtration, washed with hexanes (2×10 mL), and dried under vacuum to provide title compound (0.065 g, 18.10%) ¹H NMR (400 MHz, DMSO-d₆) δ ppm 9.29 (s, 1H), 8.55-8.58 (t, 1H), 7.26-7.30 (t, 2H), 7.16-7.20 (m, 4H), 7.00-7.02 (d, 2H), 6.93-6.95 (d, 2H), 6.65-6.68 (d, 2H), 5.07 (s, 1H), 4.11-4.22 (m, 2H), 2.22 (s, 3H), 2.15 (s, 3H).

### (c) 2-(2,4-dimethylphenyl)-N-(4-((2-methylpyridin-3-yl)methoxy)benzyl)-2-phenylacetamide

To the solution of 2-(2,4-dimethylphenyl)-N-(4-hydroxybenzyl)-2-phenylacetamide (52 mg, 0.15 mmol) and 3-(chloromethyl)-2-methylpyridine hydrochloride (32 mg, 0.18 mmol) in DMF (5 mL) was added K₂CO₃ (62 mg, 0.45 mmol). The resulting mixture was stirred at rt for 48 h. Then the mixture was poured into water and extracted with EtOAc (2×5 mL). The combined organic layers were washed with brine and dried over Na₂SO₄. After removal of solvent, the residue was purified by preparatory TLC on silica gel to afford the title compound (20 mg, 30%) as a white solid. MS: 451 [M+H]⁺; ¹H NMR (400 MHz, DMSO-d₆) δ ppm 8.63 (t, J=5.6 Hz, 1H), 8.40 (dd, J=1.2 Hz, 5.2 Hz, 1H), 7.77-7.75 (m, 1H), 7.30-6.93 (m, 13H), 5.10 (s, 2H), 5.08 (s, 1H), 4.28-4.18 (m, 2H), 2.22 (s, 3H), 2.15 (s, 3H).

### Example 13

### N-((4-chloro-2-methylphenyl)(phenyl)methyl)-2-(4-((2-methylpyridin-3-yl)oxy)phenyl)acetamide

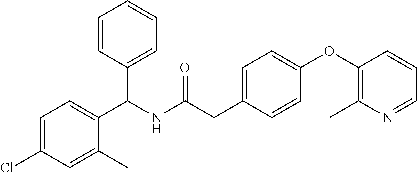 [SMILES: Cc1cc(Cl)ccc1C(NC(=O)Cc1ccc(Oc2cccnc2C)cc1)c1ccccc1]

### (a) ethyl 2-(4-((2-methylpyridin-3-yl)oxy)phenyl)acetate

To a solution of ethyl 2-(4-hydroxyphenyl)acetate (1.36 g, 7.55 mmol) in 6 mL of 1,4-dioxane were added 3-bromo-2-methylpyridine (1.0 g, 5.8 mmol), CuI (330 mg, 1.74 mmol), Cs₂CO₃ (5.67 g, 17.4 mmol) and N,N-dimethylglycine (180 mg, 1.74 mmol) under nitrogen. The resulting mixture was sealed and heated in a microwave (120° C. for 60 min). After cooling to rt, water (30 mL) was added and the reaction mixture was extracted with EtOAc (30 mL×3). The combined organic layers were washed with brine (30 mL×3), and dried over Na₂SO₄. After removal of solvent, the residue was purified by silica gel column chromatography (petroleum ether/EtOAc=5/1) to obtain title compound (600 mg, yield: 38%) as a yellow oil. LCMS-P1: 272 [M+H]⁺; Rₜ: 1.293 min.

### (b) 2-(4-((2-methylpyridin-3-yl)oxy)phenyl)acetic acid

To a solution of ethyl 2-(4-((2-methylpyridin-3-yl)oxy)phenyl)acetate (300 mg, 1.1 mmol) in THF (8 mL) and water (2 mL) was added LiOH (465 mg, 11 mmol). The mixture was stirred at rt overnight. Then water (10 mL) was added to the mixture. AcOH was used to adjust the aqueous phase to pH=3-4. Then the mixture was extracted with ethyl acetate (30 mL×3). The combined organic layers were washed with brine (15 mL×3) and dried over Na₂SO₄. The solvent was evaporated to give the title compound (210 mg, 78%) as a white solid. The title compound was used in the next step without any further purification. LCMS-P1: 244 [M+H]⁺: Rₜ: 1.082 min.

### (c) N-((4-chloro-2-methylphenyl)(phenyl)methyl)-2-(4-((2-methylpyridin-3-yl)oxy)phenyl)acetamide

This compound was synthesized from 2-(4-((2-methylpyridin-3-yl)oxy)phenyl)acetic acid and (4-chloro-2-methylphenyl)(phenyl)methanamine essentially as example 3 (42 mg, yield: 11%). LCMS-P1: 457 [M+H]⁺; Rₜ: 1.518 min. ¹H NMR (500 MHz, DMSO-d₆,) δ ppm 8.97 (d, J=8.0 Hz, 1H), 8.26 (t, J=1.5 Hz, 1H), 7.34 (t, J=7.5 Hz, 2H), 7.28-7.23 (m, 7H), 7.18 (d, J=7.5 Hz, 2H), 7.11 (d, J=8.0 Hz, 1H), 6.90 (d, J=8.5 Hz, 2H), 6.19 (d, J=8.0 Hz, 1H), 3.51 (s, 2H), 2.34 (s, 3H), 2.17 (s, 3H).

### Example 14

### N-((2,4-dimethylphenyl)(phenyl)methyl)-2-(3-methyl-4-((2-methylpyridin-3-yl)methoxy)phenyl)acetamide

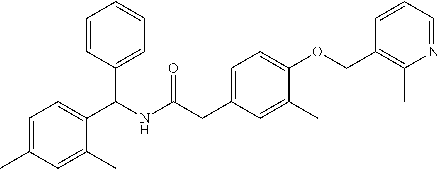 [SMILES: Cc1ccc(C(NC(=O)Cc2ccc(OCc3cccnc3C)c(C)c2)c2ccccc2)c(C)c1]

### (a) N-((2,4-dimethylphenyl)(phenyl)methyl)-2-(4-methoxy-3-methylphenyl)acetamide

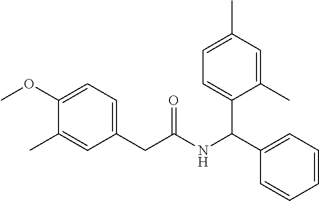 [SMILES: COc1ccc(CC(=O)NC(c2ccccc2)c2ccc(C)cc2C)cc1C]

This compound was synthesized from (2,4-dimethylphenyl)(phenyl)methanamine and 2-(4-methoxy-3-methylphenyl)acetic acid essentially as example 3 which was used in the next step without any further purification. LCMS-P1: 374 [M+H]⁺; Rₜ: 1.805 min.

### (b) N-((2,4-dimethylphenyl)(phenyl)methyl)-2-(4-hydroxy-3-methylphenyl)acetamide

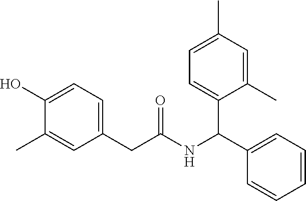 [SMILES: Cc1ccc(C(NC(=O)Cc2ccc(O)c(C)c2)c2ccccc2)c(C)c1]

To a solution of N-((4-chloro-2-methylphenyl)(phenyl)methyl)-2-(4-methoxy-3-methylphenyl)acetamide (200 mg, 0.5 mmol) in 10 mL CH₂Cl₂ was added boron tribromide (215 mg, 0.8 mmol) under nitrogen at −78° C. The mixture was allowed to warm to rt slowly and stirred overnight. The solvent was evaporated to give the title compound (162 mg, 85%) as a yellow solid. The title compound was used in the next step without further purification. LCMS-P1: 360 [M+H]⁺; Rₜ: 1.696 min.

### (c) N-((2,4-dimethylphenyl)(phenyl)methyl)-2-(3-methyl-4-((2-methylpyridin-3-yl)methoxy)phenyl)acetamide

To a stirred solution of N-((2,4-dimethylphenyl)(phenyl)methyl)-2-(4-hydroxy-3-methylphenyl)acetamide (162 mg, 0.45 mmol) in CH₃CN (20 mL) was added 3-(chloromethyl)-2-methylpyridine (89 mg, 0.54 mmol), and K₂CO₃ (186 mg, 1.35 mmol). The resulting mixture was stirred at 50° C. overnight. After cooling to rt, the solid was separated by filtration. The filtrate was concentrated and the title compound was purified by preparatory TLC on silica gel (petroleum ether/EtOAc=3/2) to obtain title compound (86 mg, yield: 41%) as a white solid. LCMS-P1: 465 [M+H]⁺; Rₜ: 1.602 min. ¹H NMR (400 MHz, DMSO-d₆) δ ppm 8.83 (d, J=8.4 Hz, 1H), 8.40-8.38 (m, 1H), 7.79-7.77 (m, 1H), 7.32-7.22 (m, 4H), 7.15 (d, J=7.2 Hz, 2H), 7.04-6.95 (m, 6H), 6.17 (d, J=8.0 Hz, 1H), 5.09 (s, 2H), 3.40 (s, 2H), 2.49 (s, 3H), 2.22 (s, 3H), 2.14 (s, 3H), 2.13 (s, 3H).

### Example 15

### N-((4-chloro-2-methylphenyl)(phenyl)methyl)-2-(4-(((2-methylpyridin-3-yl)methyl)amino)piperidin-1-yl)acetamide

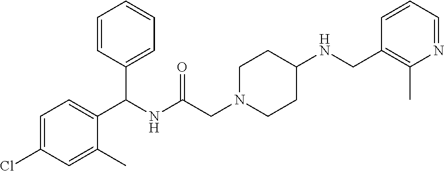 [SMILES: Cc1cc(Cl)ccc1C(NC(=O)CN1CCC(NCc2cccnc2C)CC1)c1ccccc1]

### (a) methyl 2-(4-((tert-butoxycarbonyl)amino)piperidin-1-yl)acetate

To the solution of tert-butyl piperidin-4-ylcarbamate (1 g, 5 mmol) in DMF (10 mL) were added methyl-2-bromoacetate (842 mg, 5.5 mmol) and K₂CO₃ (1.38 g, 10 mmol) and stirred at rt for 30 min. Water (10 mL) was added to the reaction mixture, and the mixture was extracted with EtOAc. The combined organic layers were washed with brine, and dried over Na₂SO₄. After removal of solvent, the title compound (1.3 g, 96%) was used in the next step without further purification. LCMS-P1: 273, [m+H]⁺.

### (b) methyl 2-(4-aminopiperidin-1-yl)acetate hydrochloride

To the solution of methyl 2-(4-((tert-butoxycarbonyl)amino)piperidin-1-yl)acetate (272 mg, 1 mmol) in MeOH (5 mL) was added 1 M HCl in Et₂O (4 mL, 4 mmol). Then the mixture was stirred at rt for 3 h. The reaction mixture was evaporated in vacuum. The title compound (239 mg, 98%) was used in the next step without further purification. MS: 173 [M+H]⁺;

### (c) methyl 2-(4-(((2-methylpyridin-3-yl)methyl)amino)piperidin-1-yl)acetate

To the mixture of methyl 2-(4-aminopiperidin-1-yl)acetate hydrochloride (190 mg, 0.78 mmol) and 3-(chloromethyl)-2-methylpyridine hydrochloride (151 mg, 0.85 mmol) in DMF (3 mL) was added K₂CO₃ (428 mg, 3.1 mmol) and potassium iodide (64.3 mg, 0.38 mmol), then the mixture was heated at 60° C. overnight under argon protection. The reaction mixture was cooled to rt and poured into water (10 mL), and extracted with EtOAc. The combined organic layers were washed with brine, and dried over Na₂SO₄. The organic solvents were removed under vacuum, and the residue was purified by silica gel column chromatography (0-100% ethyl acetate/hexanes) to give the title compound as a colorless oil (100 mg, 46.3%) MS: 278 [M+H]⁺;

### (d) 2-(4-(((2-methylpyridin-3-yl)methyl)amino)piperidin-1-yl)acetic acid

To the suspension of methyl 2-(4-(((2-methylpyridin-3-yl)methyl)amino)piperidin-1-yl)acetate (100 mg, 0.36 mmol) in THF/Water (4 mL, 1:1) was added LiOH (30 mg, 0.72 mmol) and the resulting mixture was stirred at rt for 30 min. The reaction mixture was neutralized by HCl (1M). All the solvent was removed under reduced pressure and the title compound was used directly to the next step without further purification. MS: 264 [M+H]⁺;

### (e) N-((4-chloro-2-methylphenyl)(phenyl)methyl)-2-(4-(((2-methylpyridin-3-yl)methyl)amino)piperidin-1-yl)acetamide

To the solution of 2-(4-(((2-methylpyridin-3-yl)methyl)amino)piperidin-1-yl)acetic acid (crude product) in DMF (3 mL) were added (4-chloro-2-methylphenyl)(phenyl)methanamine hydrochloride (116 mg, 0.43 mmol), EDC (83 mg, 0.43 mmol), HOBt (58 mg, 0.43 mmol), and DIPEA (93 mg, 0.72 mmol). The resulting mixture was heated at 45° C. overnight. After cooling to rt, the mixture was extracted with EtOAc (15 mL×2). The combined organic layers were washed with brine and dried over Na₂SO₄. After removal of solvent, the residue was purified by preparatory HPLC using 10-100% water/acetonitrile with 0.1% TFA to afford the title compound as a white solid 31 mg, 18%. MS: 477 [M+H]⁺; ¹H NMR (400 MHz, DMSO-d₆) δ ppm 8.46 (d, J=8.4 Hz, 1H), 8.27 (dd, J=4 Hz, 1H), 7.66 (d, J=7.6 Hz, 1H), 7.34-7.14 (m, 9H), 6.23 (d, J=8.8 Hz, 1H), 3.67 (s, 2H), 2.99 (s, 2H), 2.77-2.74 (m, 2H), 2.46 (s, 3H), 2.23 (s, 3H), 2.09 (t, J=7.2 Hz, 2H), 1.81 (d, J=12 Hz, 2H), 1.32-1.23 (m, 2H).

### Example 16

### N-((2,4-dimethylphenyl)(phenyl)methyl)-2-(4-methyl-4-(((2-methylpyridin-3-yl)methyl)amino)piperidin-1-yl)acetamide

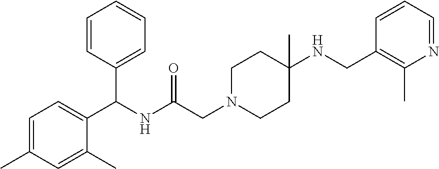 [SMILES: Cc1ccc(C(NC(=O)CN2CCC(C)(NCc3cccnc3C)CC2)c2ccccc2)c(C)c1]

### (a) 1-tert-butyl 4-methyl piperidine-1,4-dicarboxylate

To the suspension of 1-(tert-butoxycarbonyl)piperidine-4-carboxylic acid (4.58 g, 20 mmol) and K₂CO₃ (5.26 g, 40 mmol) in DMF (20 mL) was added iodomethane (4.26 g, 30 mmol) slowly. Then the resulting mixture was stirred at rt overnight under argon. The solid was removed by filtration. The resulting filtrate was extracted with EtOAc. The combined organic solvents were washed with brine and dried with Na₂SO₄. After removal of solvent, the crude title compound (4.7 g, 98%) was used in the next step without further purification. MS: 244.0, [M+H]⁺.

### (b) 1-tert-butyl 4-methyl 4-methylpiperidine-1,4-dicarboxylate

To the solution of 1-tert-butyl 4-methyl piperidine-1,4-dicarboxylate (607 mg, 2.5 m mol) in THF (5 mL) was added 2 M lithium diisopropylamine (1.5 mL, 3 mmol) dropwise at −78° C. under argon protection. After stirring at −78° C. for 1 hour, iodomethane (426 mg, 3 mmol) was added. The resulting mixture was allowed to warm up to n slowly, and stirred at rt overnight. The reaction mixture was quenched with NH₄Cl (sat.), extracted with EtOAc, and the combined organic layers were washed with brine and dried over Na₂SO₄. After removal of solvent, the residue was purified by gel silica column chromatography with petroleum ether:EtOAc=5:1 to afford the title compound as a colorless oil (274 mg, 43%). MS: 258.0 [M+H]⁺;

### (c) 1-(tert-butoxycarbonyl)-4-methylpiperidine-4-carboxylic acid

To the suspension of 1-tert-butyl 4-methyl 4-methylpiperidine-1,4-dicarboxylate (274 mg, 1.07 mmol) in water/THF (4 mL, 1:1) was added LiOH:H₂O (89 mg, 2.13 mmol), and then the mixture was stirred at 45° C. overnight. The reaction mixture was cooled to rt and the pH was adjusted to 5-6 by using 2 N HCl. Then the mixture was extracted with EtOAc. The combined organic layers were washed with brine and dried over Na₂SO₄. After removal of solvent, the crude title compound (145 mg, 56%) was used in the next step without further purification. MS: 244.0 [M+H]⁺;

### (d) tert-butyl 4-(((benzyloxy)carbonyl)amino)-4-methylpiperidine-1-carboxylate

To the solution of 1-(tert-butoxycarbonyl)-4-methylpiperidine-4-carboxylic acid (141 mg, 0.58 mmol) in toluene (3 mL) was added triethylamine (88 mg, 0.87 mmol), diphenyl phosphorazidate (313 mg, 0.81 mmol), and phenylmethanol (313 mg, 12.9 mmol). The mixture was stirred at rt for 1 hour, and then heated to 80° C. under argon protection overnight. After cooling to rt, the solvent was removed under reduced pressure, the residue was purified by gel silica column chromatography with petroleum ether:EtOAc=5:1 to afford the title compound as colorless oil 90 mg, 44.50%. MS: 349.0 [M+H]⁺;

### (e) benzyl (4-methylpiperidin-4-yl)carbamate

To a solution of tert-butyl 4-(((benzyloxy)carbonyl)amino)-4-methylpiperidine-1-carboxylate (1.1 g, 3.7 mmol) in 1,4-dioxane (2 mL) was added HCl/1,4-dioxane (8 mL, 4N, 32 mmol), the resulting mixture was heated to 50° C. overnight. After cooling to rt, the solvent was removed under reduced pressure. To the residue was added EtOAc and acetone. The precipitate was collected by filtration to give the title compound as a white solid (550 mg, 70%). MS: 249 [M+H]⁺;

### (f) ethyl 2-(4-(((benzyloxy)carbonyl)amino)-4-methylpiperidin-1-yl)acetate

To the solution of benzyl (4-methylpiperidin-4-yl)carbamate (124 mg, 0.5 mmol) in DMF (3 mL) was added ethyl 2-bromoacetate (100 mg, 0.6 mmol) and K₂CO₃ (138 mg, 1 mmol). Then the mixture was heated to 60° C. for 3 h. After cooling to rt, the reaction mixture was poured into water and extracted with EtOAc. The combined organic layers were washed with brine and dried over Na₂SO₄. After removal of solvent, the residue was purified by gel silica column chromatography with petroleum ether:EtOAc=1:1 to afford the title compound as colorless oil 100 mg, 60%. MS: 335 [M+H]⁺;

### (g) ethyl 2-(4-amino-4-methylpiperidin-1-yl)acetate

To the solution of ethyl 2-(4-(((benzyloxy)carbonyl)amino)-4-methylpiperidin-1-yl)acetate (110 mg, 0.33 mmol) in EtOAc (5 mL) was added 10% Pd/C (11 mg), then the mixture was stirred at rt under hydrogen atmosphere overnight. Pd/C was removed by filtration. The organic layer was removed under reduced pressure to give the titled compound as a colorless oil (60 mg, 91%). MS: 201 [M+H]⁺;

### (h) ethyl 2-(4-methyl-4-(((2-methylpyridin-3-yl)methyl)amino)piperidin-1-yl)acetate

This compound was synthesized from ethyl 2-(4-(((benzyloxy)carbonyl)amino)-4-methylpiperidin-1-yl)acetate and 3-(chloromethyl)-2-methylpyridine hydrochloride essentially as example 15 (c) (200 mg crude product was obtained). MS: 306 [M+H]⁺

### (i) 2-(4-methyl-4-(((2-methylpyridin-3-yl)methyl)amino)piperidin-1-yl)acetic acid

This compound was synthesized from ethyl 2-(4-methyl-4-(((2-methylpyridin-3-yl)methyl)amino)piperidin-1-yl)acetate essentially as example 15 (d) (300 mg crude product was obtained). MS: 278 [M+H]⁺;

### (j) N-((2,4-dimethylphenyl)(phenyl)methyl)-2-(4-methyl-4-(((2-methylpyridin-3-yl)methyl)amino)piperidin-1-yl)acetamide

This compound was synthesized from 2-(4-methyl-4-(((2-methylpyridin-3-yl)methyl)amino)piperidin-1-yl)acetic acid and (2,4-dimethylphenyl)(phenyl)methanamine essentially as example 15 (e) (20 mg, 7% over three steps). MS: 471 [M+H]⁺; ¹H NMR (400 MHz, DMSO-d₆) δ ppm 8.32-8.27 (m 2H), 7.69 (d, J=8 Hz, 1H), 7.32 (t, J=7.2 Hz, 2H), 7.26-7.23 (m 1H), 7.18-7.14 (m 3H), 7.03-7.6.96 (m, 3H), 6.23 (d, J=8.8 Hz, 1H), 3.58 (s, 1H), 3.00 (s, H), 2.39-2.30 (m 3), 2.24 (s 3H), 2.19 (s 3H), 1.62-1.47 (m, 5H), 1.10 (s, 3H).

### Example 17

### N-((2,4-dimethylphenyl)(phenyl)methyl)-2-(4-(2-hydroxy-2-(2-methylpyridin-3-yl)ethoxy)phenyl)acetamide

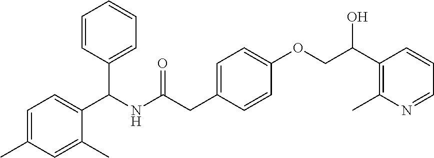 [SMILES: Cc1ccc(C(NC(=O)Cc2ccc(OCC(O)c3cccnc3C)cc2)c2ccccc2)c(C)c1]

### (a) 2-bromo-1-(2-methylpyridin-3-yl)ethanone hydrobromide

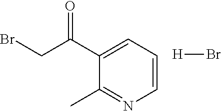

To a cooled (0° C.) solution of 1-(2-methylpyridine-3-yl)ethanone (360 mg, 2.67 mmol) in hydrogen bromide-acetic acid solution (33%, 4 mL) was added a solution of bromine (45 mg, 2.8 mmol) in dichloromethane (1 mL) slowly. The reaction mixture was allowed to rt, and the mixture was stirred at the same temperature for 2 h. The obtained solid was washed with ethyl acetate to give the title compound as a white powder (673 mg, yield: 85.5%). LCMS-P1: 215.9 [M+H]⁺; Rₜ=1.02 min.

### (b) methyl 2-(4-(2-(2-methylpyridin-3-yl)-2-oxoethoxy)phenyl)acetate

A mixture of 2-bromo-1-(2-methylpyridin-3-yl)ethanone hydrobromide (295 mg, 1 mmol), methyl 2-(4-hydroxyphenyl)acetate (199.2 mg, 1.1 mmol) and potassium carbonate (414 mg, 3 mol) in DMF (5 mL), was stirred at 50° C. overnight under argon. The reaction mixture was cooled to rt and filtered. The filtrate was dissolved in water (20 mL) and extracted by methyl tert-butyl ether (3×30 mL). The combined organic layers were dried over anhydrous sodium sulfate. and concentrated under reduced pressure and purified by column chromatography on silica gel (petroleum ether/EtOAc=2/1) to obtain title compound (141 mg, yield: 47%). ¹H NMR (400 MHz, DMSO-d₆) δ ppm 8.61 (dd, J=1.6 Hz, 5.2 Hz, 1H), 8.29 (dd, J=1.6 Hz, 5.7 Hz, 2H), 7.41 (dd, J=5.7 Hz, 7.6 Hz, 1H), 7.17 (d, J=8.0 Hz, 2H), 6.91 (d, J=8.0 Hz, 1H), 5.42 (s, 2H), 3.60 (s, 5H), 2.58 (s, 3H).

### (c) 2-(4-(2-(2-methylpyridin-3-yl)-2-oxoethoxy)phenyl)acetic acid

A mixture of methyl 2-(4-(2-(2-methylpyridin-3-yl)-2-oxoethoxy)phenyl)acetate (141 mg, 0.472 mmol) and NaOH (94 mg, 2.55 mmol) in THF:H₂O (10:2 mL) was stirred at rt for 1 h. The reaction mixture was extracted by methyl tert-butyl ether (3×10 mL) and the aqueous phase was adjusted pH to 5 with cone. HCl. The aqueous layer was then extracted with ethyl acetate (3×30 mL), and the combined organic layers were dried over anhydrous sodium sulfate was and concentrated under reduced pressure to obtain the title compound (90 mg, yield: 64%). LCMS-P1: 286.0 [M+H]⁺; Rₜ=1.096 min.

### (d) N-((2,4-dimethylphenyl)(phenyl)methyl)-2-(4-(2-(2-methylpyridin-3-yl)-2-oxoethoxy)phenyl)acetamide

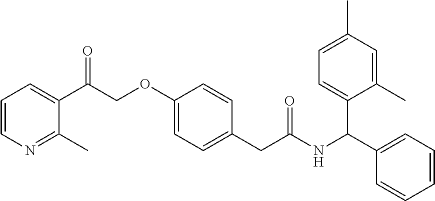 [SMILES: Cc1ccc(C(NC(=O)Cc2ccc(OCC(=O)c3cccnc3C)cc2)c2ccccc2)c(C)c1]

A mixture of 2-(4-(2-(2-methylpyridin-3-yl)-2-oxoethoxy)phenyl)acetic acid (60 mg, 0.21 mmol), EDC (80.8 mg, 0.42 mmol), and HOBt (28.6 mg, 0.25 mmol) in dichloromethane (2 mL), was stirred at rt for 0.5 h. Then (2,4-dimethylphenyl)(phenyl)methanamine (53 mg, 0.21 mmol) was added. After the addition, the mixture was stirred at rt overnight. The reaction mixture was washed with water (3×1 mL), the organic layer dried over anhydrous sodium sulfate and concentrated under reduced pressure and purified by column chromatography on silica gel (petroleum ether/EtOAc=1:1) to obtain title compound (45 mg, 45%). ¹H NMR (400 MHz, DMSO-d₆) δ ppm 8.82 (d, =8.4 Hz, 1H), 8.61 (dd, J=1.6 Hz, 4.8 Hz, 1H), 8.29 (dd, J=1.6 Hz, 4.8 Hz, 1H), 7.40 (dd, J=4.8 Hz, 7.6 Hz, 1H), 7.73-7.24 (m, 3H), 7.17-7.14 (m, 4H), 6.95 (m, 3H), 6.87 (d, J=8.8 Hz, 2H), 6.17 (d, J=8.8 Hz, 1H), 5.39 (s, 2H), 3.44 (s, 2H), 2.58 (s, 3H), 2.23 (s, 3H), 2.14 (s, 3H).

### (e) N-((2,4-dimethylphenyl)(phenyl)methyl)-2-(4-(2-hydroxy-2-(2-methylpyridin-3-yl)ethoxy)phenyl)acetamide

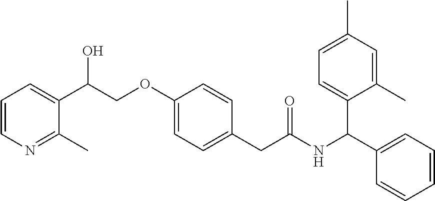 [SMILES: Cc1ccc(C(NC(=O)Cc2ccc(OCC(O)c3cccnc3C)cc2)c2ccccc2)c(C)c1]

To a 0° C. solution of N-((2,4-dimethylphenyl)(phenyl)methyl)-2-(4-(2-(2-methylpyridin-3-yl)-2-oxoethoxy)phenyl)acetamide (35 mg, 0.073 mmol) in MeOH (4 mL) was added NaBH₄ (5.6 mg, 0.146 mmol). The reaction mixture was stirred at rt for 2 h. Then water (2 mL) was added to the mixture and concentrated to dryness. The residue was purified by column chromatography on silica gel (petroleum ether/EtOAc=1/1) to obtain title compound as a white solid (18 g, yield: 51%). LCMS-P1: 481.2 [M+H]⁺; Rₜ=1.93 min. ¹H NMR (400 MHz, DMSO-d₆) δ ppm 8.81 (d, J=8.0 Hz, 1H), 8.35 (dd, J=1.6 Hz, 4.8 Hz, 1H), 7.85 (dd, J=1.6 Hz, 6 Hz, 1H), 7.30 (t, J=7.2 Hz, 2H), 7.25 (m, 2H), 7.16 (s, 2H), 7.14 (s, 2H), 6.97-6.97 (m, 3H), 6.86 (s, 1H), 6.84 (s, 1H), 6.17 (d, J=8.8 Hz, 1H), 5.71 (d, J=4.8 Hz 1H), 5.10 (q, J=4.8 Hz, 1H), 3.99 (d, J=6.0 Hz, 2H), 3.42 (s, 2H), 2.54 (s, 3H), 2.23 (s, 3H), 2.14 (s, 3H).

Following essentially the procedure as described in Example 1, the compounds in Table 1 were prepared.

TABLE 1

| Ex. | Structure/Name | NMR | LCMS |
|---|---|---|---|
| 18 | 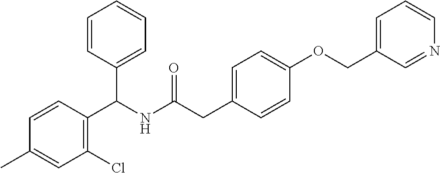 [SMILES: Cc1ccc(C(NC(=O)Cc2ccc(OCc3cccnc3)cc2)c2ccccc2)c(Cl)c1] N-((2-chloro-4- methylphenyl)(phenyl)methyl)-2-(4- (pyridin-3-ylmethoxy)phenyl)acetamide | ¹H NMR (400 MHz, DMSO-d₆) δ ppm 8.94-8.96 (d, 1 H), 8.65- 8.66 (d, 1 H), 8.53-8.55 (dd, 1 H), 7.84-7.86 (d, 1 H), 7.40- 7.43 (dd, 1 H), 7.23-7.33 (m, 5 H), 7.13-7.20 (m, 5 H), 6.94- 6.96 (d, 2 H), 6.33-6.35 (d, 1 H), 5.12 (s, 2 H), 3.45 (s, 2 H), 2.28 (s, 3 H). | LCMS-X1: 457.6 [M + H]⁺; Rₜ = 5.84 min |
| 19 | 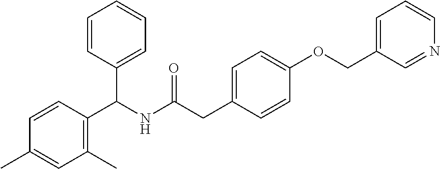 [SMILES: Cc1ccc(C(NC(=O)Cc2ccc(OCc3cccnc3)cc2)c2ccccc2)c(C)c1] N-((2,4-dimethylphenyl)(phenyl)methyl)- 2-(4-(pyridin-3-ylmethoxy)phenyl) acetamide | ¹H NMR (400 MHz, DMSO-d₆) δ ppm 8.81-8.83 (d, 1 H), 8.662-8.666 (d, 1 H), 8.53-8.55 (q, 1 H), 7.84-7.86 (d, 1 H), 7.40-7.44 (m, 1 H), 7.29-7.32 (t, 2 H), 7.22-7.26 (t, 1 H), 7.14-7.20 (q, 4 H), 6.94-6.97 (m, 5 H), 6.16-6.18 (d, 1 H), 5.13 (s, 2 H), 3.44 (s, 2 H), 2.23 (s, 3 H), 2.14 (s, 3H). | LCMS-X1: 437.4 [M + H]⁺; Rₜ = 5.83 min |
| 20 | 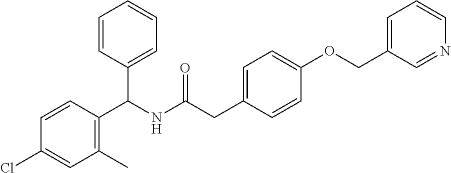 [SMILES: Cc1cc(Cl)ccc1C(NC(=O)Cc1ccc(OCc2cccnc2)cc1)c1ccccc1] N-((4-chloro-2- methylphenyl)(phenyl)methyl)-2-(4- (pyridin-3-ylmethoxy)phenyl)acetamide | ¹H NMR (400 MHz, DMSO-d₆) δ ppm 8.90-8.92 (d, 1 H), 8.662-8.666 (d, 1 H), 8.53-8.55 (q, 1 H), 7.85-7.87 (d, 1 H), 7.40-7.44 (m, 1 H), 7.31-7.35 (t, 2 H), 7.24-7.28 (m, 3 H), 7.16-7.20 (t, 4 H), 7.09-7.11 (d, 1 H), 6.94-6.96 (d, 2 H), 6.16- 6.18 (d, 1 H), 5.13 (s, 2H), 3.44-3.49 (d, 2 H), 2.50 (s , 3 H). | LCMS-X1: 457.3 [M + H]⁺; Rₜ = 5.99 min |

Following essentially the procedure as described in Example 2, the compounds in Table 2 were prepared.

TABLE 2

| Ex. | Structure/Name | NMR | LCMS |
|---|---|---|---|
| 21 | 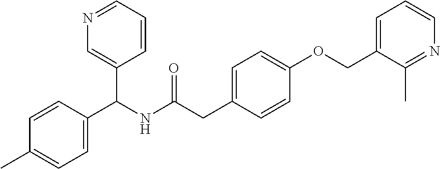 [SMILES: Cc1ccc(C(NC(=O)Cc2ccc(OCc3cccnc3C)cc2)c2cccnc2)cc1] 2-(4-((2-methylpyridin-3- yl)methoxy)phenyl)-N-(pyridin)-3-yl(p- tolyl)methyl)acetamide | ¹H NMR (400 MHz, DMSO- d₆) δ ppm 9.98 (d, J = 8.4 Hz, 1H), 8.48-8.38 (m, 3H), 7.77- 7.76 (m, 1H), 7.75-7.74 (m, 1H), 7.62-7.59 (m, 1H), 7.34- 7.11 (m, 7H), 6.97-6.95 (m, 2H), 6.09 (d, J = 8.0 Hz, 1H), 5.08 (s, 2H), 3.48 (s, 2H), 2.49 (s, 3H), 2.25 (s, 3H). | LCMS-P1: 438 [M + H]⁺; Rₜ = 1.488 min. |
| 22 | 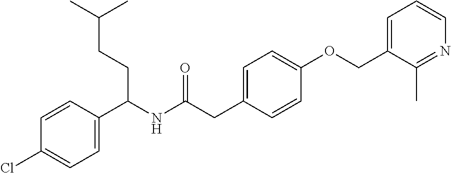 [SMILES: Cc1ncccc1COc1ccc(CC(=O)NC(CCC(C)C)c2ccc(Cl)cc2)cc1] N-(1-(4-chlorophenyl)-4-methylpentyl)-2- (4-((2-methylpyridin-3- yl)methoxy)phenyl)acetamide | ¹H NMR (400 MHz, DMSO- d₆) δ ppm 8.46 (d, J = 8.4 Hz, 1H), 8.39-8.38 (m, 1H), 7.76- 7.74 (m, 1H), 7.35-7.27 (m, 4H), 7.23-7.20 (m, 1H), 7.16 (d, J = 8.4 Hz, 2H), 6.95 (d, J = 8.4 Hz, 2H), 5.08 (s, 2H), 4.70-4.64 (m, 1H), 3.40-3.32 (m, 2H), 2.48 (s, 3H), 1.64- 1.57 (m, 2H), 1.49-1.42 (m, 1H), 1.15-1.00 (m, 2H), 0.79 (t, J = 6.8 Hz, 6H). | LCMS-P1: 451 [M + H]⁺; Rₜ = 1.775 min. |
| 23 | 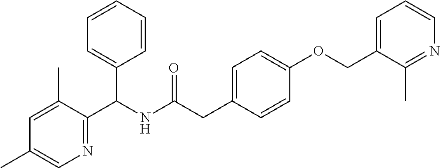 [SMILES: Cc1cnc(C(NC(=O)Cc2ccc(OCc3cccnc3C)cc2)c2ccccc2)c(C)c1] N-((3,5-dimethylpyridin-2-yl)(phenyl)- methyl)-2-(4-((2-methylpyridin-3- yl)methoxy)phenyl)acetamide | ¹H NMR (MeOH-d₄, 400 MHz) δ ppm 8.37 (m, 1H), 8.23 (m, 1H), 7.88 (m, 1H), 7.40 (m, 1H), 7.20-7.30 (m, 8H), 6.98 (m, 2H), 6.27 (s, 1H), 5.12 (s, 2H), 3.56 (s, 2H), 2.58 (s, 3H), 2.31 (m, 3H), 2.21 (s, 3H). | LCMS-P1: 452.2, [M + H]⁺. Rₜ = 1.96 min |
| 24 | 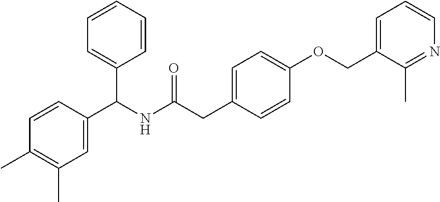 [SMILES: Cc1ccc(C(NC(=O)Cc2ccc(OCc3cccnc3C)cc2)c2ccccc2)cc1C] N-((3,4-dimethylphenyl)(phenyl)methyl)- 2-(4-((2-methylpyridin-3- yl)methoxy)phenyl)acetamide | ¹H NMR (400 MHz, CDCl₃) δ ppm 8.50 (s, 1H), 7.74 (d, J = 7.2 Hz, 1H), 7.31-6.81 (m, 13H), 6.18 (d, J = 8.0 Hz, 1H), 6.01 (d, J = 8.0 Hz, 1H), 5.05 (s, 2H), 3.6 (s, 2H), 2.61 (s, 3H), 2.22 (s, 3H), 2.19 (s, 3H). | LCMS-P1 : 451 [M + H]⁺; Rₜ = 1.702 min. |

Following essentially the procedure as described in Example 5, the compounds in Table 3 were prepared.

TABLE 3

| Ex. | Structure/Name | NMR | LCMS |
|---|---|---|---|
| 25 | 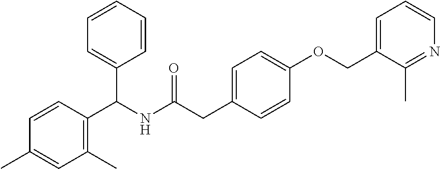 [SMILES: Cc1ccc(C(NC(=O)Cc2ccc(OCc3cccnc3C)cc2)c2ccccc2)c(C)c1] N-((2,4-dimethylphenyl)(phenyl)methyl)- 2-(4-((2-methylpyridin-3- yl)methoxy)phenyl)acetamide | ¹H NMR (400 MHz, DMSO-d₆) δ ppm 8.83-8.85 (d, 1 H), 8.39- 8.41 (dd, 1 H), 7.76-7.78 (d, 1 H), 7.26-7.33 (m, 2 H), 7.15- 7.25 (m, 6 H), 6.94-6.98 (m, 5 H), 6.16-6.18 (d, 1 H), 5.10 (s, 2 H), 3.45 (s, 2 H), 2.51 (s, 3 H), 2.23 (s, 3H), 2.15 (s, 3H). | LCMS-X1: 451.7 [M + H]⁺; Rₜ = 5.54 min |

| | | | |
|---|---|---|---|
| 26 | 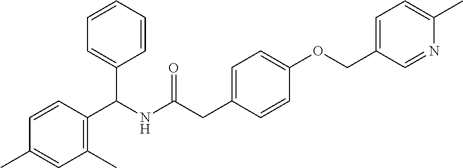 N-((2,4-dimethylphenyl)(phenyl)methyl)- 2-(4-((6-methylpyridin-3- yl)methoxy)phenyl)acetamide | ¹H NMR (400 MHz, DMSO-d₆) δ ppm 8.81-8.83 (d, 1 H), 8.51- 8.52 (d, 1 H), 7.72-7.74 (dd, 1 H), 7.22-7.32 (m, 8 H). 6.92- 6.98 (m, 5 H), 6.16-6.18 (d, 1 H), 5.07 (s, 2 H), 3.57 (s, 2 H), 2.50 (s, 3 H), 2.28 (s, 3 H), 2.14 (s, 3 H). | LCMS-X1: 451.7 [M + H]⁺; Rₜ = 5.62 min |
| 27 | 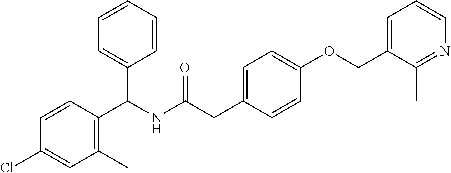 [SMILES: Cc1cc(Cl)ccc1C(NC(=O)Cc1ccc(OCc2cccnc2C)cc1)c1ccccc1] N-((4-chloro-2-methylphenyl)(phenyl)- methyl)-2-(4-((2-methylpyridin-3- yl)methoxy)phenyl)acetamide | ¹H NMR (400 MHz, DMSO-d₆) δ ppm 8.91-8.93 (d, 1 H), 8.40- 8.41 (dd, 1 H), 7.77-7.79 (d, 1 H), 7.31-7.35 (m, 2 H), 7.20- 7.28 (m, 8 H), 7.09-7.11 (d, 1 H), 6.96-6.98 (d, 2 H), 6.17- 6.19 (d, 1 H), 5.10 (s, 2 H), 3.45 (s, 2 H), 2.50 (s, 3 H), 2.18 (s, 3 H). | LCMS-X1: 470.5 [M + H]⁺; Rₜ = 5.68 min |
| 28 | 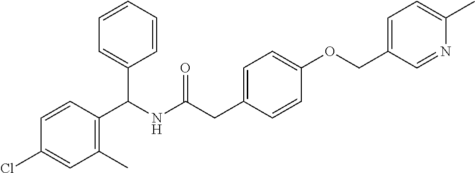 [SMILES: Cc1ccc(COc2ccc(CC(=O)NC(c3ccccc3)c3ccc(Cl)cc3C)cc2)cn1] N-((4-chloro-2-methylphenyl)(phenyl)- methyl)-2-(4-((6-methylpyridin-3- yl)methoxy)phenyl)acetamide | ¹H NMR (400 MHz, DMSO-d₆) δ ppm 8.90-8.92 (d, 1 H), 8.51- 8.52 (d, 1 H), 7.73-7.75 (dd, 1 H), 7.33-7.43 (m, 2 H), 7.28- 7.31 (m, 4 H), 7.17-7.22 (m, 4 H), 7.08-7.10 (d, 1 H), 6.92- 6.94 (d, 2 H), 6.16-6.18 (d, 1 H), 5.07 (s, 2 H), 3.40 (s, 2 H), 2.47 (s, 3 H), 2.18 (s, 3H). | LCMS-X1: 470.7 [M + H]⁺; Rₜ = 5.72 min |

Following essentially the procedure as described in Example 9, the compound in Table 4 was prepared.

TABLE 4

| Ex. | Structure/Name | NMR | LCMS |
|---|---|---|---|
| 29 | 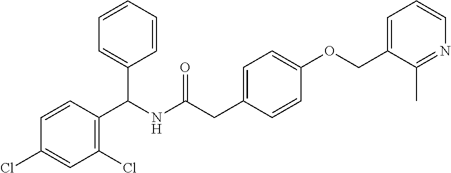 [SMILES: Cc1ncccc1COc1ccc(CC(=O)NC(c2ccccc2)c2ccc(Cl)cc2Cl)cc1] N-((2,4-dichlorophenyl)(phenyl)methyl)- 2-(4-((2-methylpyridin-3- yl)methoxy)phenyl)acetamide | 1H NMR (400 MHz, CDCl₃) δ ppm 8.50-8.48 (dd, J = 4.8 Hz, 1.6 Hz, 1H), 7.74 -7.72 (dd, J = 7.6 Hz, 1.5 Hz, 1H), 7.38-7.37 (d, J = 2.1 Hz, 1H), 7.30-7.28 (m, 2H), 7.24-7.20 (m, 3H), 7.19-7.16 (m, 1H), 7.11-7.08 (d, J = 8.5 Hz, 1H), 7.06-7.04 (m, 2H), 6.99-6.97 (d, J = 8.8 Hz, 2H), 6.46-6.44 (d, J = 7.7 Hz, 1H), 6.11-6.09 (d, J = 8.0 Hz, 1H), 5.06 (s, 2H), 3.62 (s, 2H), 2.61 (s, 3H) | LCMS-G30: 490.8 [M + H]⁺; Rₜ = 10.82 min |
| 30 | 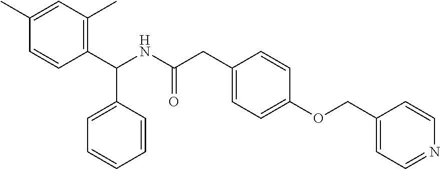 [SMILES: Cc1ccc(C(NC(=O)Cc2ccc(OCc3ccncc3)cc2)c2ccccc2)c(C)c1] N-((2,4-dimethylphenyl)(phenyl)methyl)- 2-(4-(pyridin-4- ylmethoxy)phenyl)acetamide | ¹H NMR (DMSO-d₆, 400 MHz): δ 8.85 (d, J = 8.0 Hz, 1H), 8.75 (s, 2H), 7.74 (d, J = 4.4 Hz, 2H), 7.32-7.15 (m, 7H), 6.97 (s, 5H), 6.17 (d, J = 8.0 Hz, 1H), 5.31 (s, 2H), 3.45 (s, 2H), 2.23 (s, 3H), 2.14 (s, 3H). | LCMS- A024: 437.2 [M + H]⁺; Rt: 1.33 min |
| 31 | 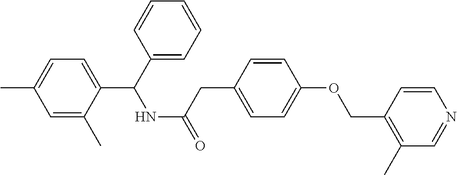 [SMILES: Cc1ccc(C(NC(=O)Cc2ccc(OCc3ccncc3C)cc2)c2ccccc2)c(C)c1] N-((2,4-dimethylphenyl)(phenyl)methyl)- 2-(4-((3-methylpyridin-4- yl)methoxy)phenyl)acetamide | ¹H NMR (CDCl₃, 400 MHz): δ 8.49-8.46 (m, 2H), 7.48 (d, J = 4.4 Hz, 1H), 7.24-7.21 (m, 4H), 7.06 (d, J = 6.8 Hz, 2H), 6.98- 6.91 (m, 4H), 6.78 (d, J = 8.0 Hz, 1H), 6.36 (d, J = 8.4 Hz, 1H), 5.89 (d, J = 8.0 Hz, 1H), 5.06 (s, 2H), 3.59 (s, 2H), 2.36 (s, 3H), 2.29 (s, 3H), 2.21 (s, 3H). | LCMS- A012: 451.0 [M + H]⁺; Rt = 1.854 min. |

### Example 32

### N-((2,4-dimethylphenyl)(phenyl)methyl)-2-(4-(1-hydroxy-2-(2-methylpyridin-3-yl)ethyl)phenyl)acetamide

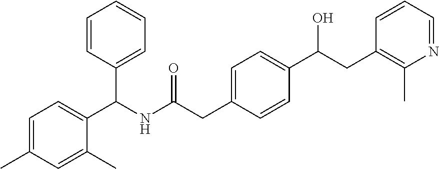 [SMILES: Cc1ccc(C(NC(=O)Cc2ccc(C(O)Cc3cccnc3C)cc2)c2ccccc2)c(C)c1]

### (a) Ethyl 2-(4-((trimethylsilyl)ethynyl)phenyl)acetate

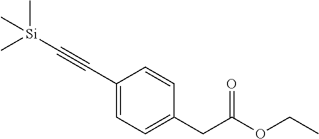 [SMILES: CCOC(=O)Cc1ccc(C#C[Si](C)(C)C)cc1]

Ethynyltrimethylsilane (2.15 g, 21.42 mmol) was added drop-wise to a solution of ethyl 2-(4-bromophenyl)acetate (5 g, 19.8 mmol), CuI (385 mg, 1.98 mmol) and Pd(PPh₃)₂Cl₂ (1.42 g, 1.98 mmol) in 120 mL of Et₃N at 0° C. under nitrogen atmosphere. The resulting mixture was refluxed overnight and Et₃N was removed under reduced pressure to give a crude solid which was purified by silica gel column chromatography (Petroleum ether/EtOAc=10/1) to obtain title compound (2.6 g, yield: 50.5%). LCMS-A024: 261.7 [M+H]⁺; Rt=2.26 min.

### (b) methyl 2-(4-ethynylphenyl)acetate

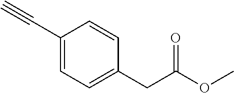

K₂CO₃ (0.223 g, 16 mmol) was added to a stirred solution of ethyl 2-(4-((trimethylsilyl)ethynyl)phenyl)acetate (2.6 g, 8 mmol) in 30 mL of MeOH at 0° C. The resulting mixture was stirred at rt for 1 h. Then water (200 mL) was added. The mixture was extracted with CH₂Cl₂ (3×300 mL) and dried over Na₂SO₄. After removal of the solvent under reduced pressure, the crude product was purified by flash chromatography (Petroleum ether/EtOAc=5/1) to give title compound (1.4 g, 7.5 mmol, yield: 93.8%). LCMS-A024: 175.1 [M+H]⁺; Rt=1.66 min.

### (c) methyl 2-(4-((2-methylpyridin-3-yl)ethynyl)phenyl)acetate

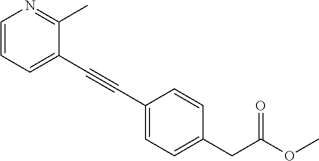 [SMILES: COC(=O)Cc1ccc(C#Cc2cccnc2C)cc1]

Methyl 2-(4-ethynylphenyl)acetate (0.9 g, 4.39 mmol) was added drop-wise to a stirred solution of 3-bromo-2-methylpyridine (0.8 g, 4.39 mmol), CuI (89 mg, 0.44 mmol), and Pd(PPh₃)₂Cl₂ (316 mg, 0.44 mmol) in 50 mL of Et₃N 0° C. under nitrogen atmosphere. The resulting mixture was refluxed overnight. Et₃N was removed under reduced pressure to give a crude solid which was purified by silica gel column chromatography (Petroleum ether/EtOAc=5/1) to obtain title compound (0.2 g, yield: 17.2%). LCMS-A024: 266.0 [M+H]⁺; Rt=1.31 min.

### (d) 2-(4-((2-methylpyridin-3-yl)ethynyl)phenyl)acetic acid

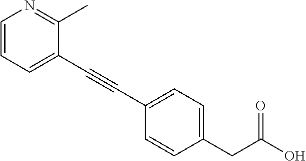 [SMILES: Cc1ncccc1C#Cc1ccc(CC(=O)O)cc1]

A solution of LiOH (71 mg, 1.7 mmol) in water (2 mL) was added to the suspension of methyl 2-(4-((2-methylpyridin-3-yl)ethynyl)phenyl)acetate (90 mg, 0.339 mmol) in THF (5 mL). After stirring at rt for 4 h, the reaction mixture was evaporated under reduced pressure to remove the excess THF. Then HCl (2M) was added slowly until a precipitate appeared. Then the precipitate was collected to give the titled compound (50 mg, yield 58.6%). LCMS-A024: 252.7 [M+H]⁺.

### (e) 2-(4-(2-(2-methylpyridin-3-yl)acetyl)phenyl)acetic acid

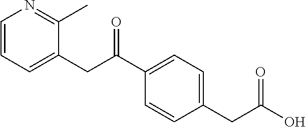 [SMILES: Cc1ncccc1CC(=O)c1ccc(CC(=O)O)cc1]

H₂SO₄ (41 mg, 0.39 mmol) was added to a solution of 2-(4-((2-methylpyridin-3-yl)ethynyl)phenyl)acetic acid (50 mg, 0.19 mmol) and HgSO₄ (59 mg, 0.19 mmol) in acetone (5 mL) and H₂O (1 mL) under nitrogen. The mixture was refluxed overnight. After cooling to rt, H₂O (20 mL) was added and the mixture was extracted with CH₂Cl₂ (3×30 mL) and dried over Na₂SO₄. After removal of the solvent, the crude product was purified by flash chromatography (Petroleum ether/EtOAc=1/1) to give title compound (20 mg, yield: 38%). LCMS-A024: 270.7 [M+H]⁺; Rt=1.02 min.

### (f) N-((2,4-dimethylphenyl)(phenyl)methyl)-2-(4-(2-(2-methylpyridin-3-yl)acetyl)phenyl)acetamide

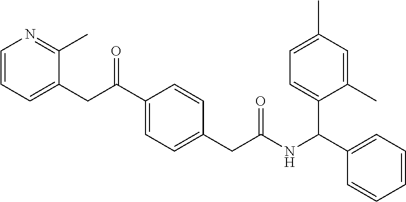 [SMILES: Cc1ccc(C(NC(=O)Cc2ccc(C(=O)Cc3cccnc3C)cc2)c2ccccc2)c(C)c1]

This compound was synthesized from 2-(4-(2-(2-methylpyridin-3-yl)acetyl)phenyl)acetic acid and (2,4-dimethylphenyl)(phenyl)methanamine essentially as described in example 2 (f) (20 mg, Yield: 64.6%). LCMS-A024: 463.7. [M+H]⁺. Rt=1.38 min

### (g) N-((2,4-dimethylphenyl)(phenyl)methyl)-2-(4-(1-hydroxy-2-(2-methylpyridin-3-yl)ethyl)phenyl)acetamide

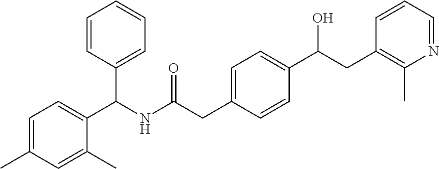 [SMILES: Cc1ccc(C(NC(=O)Cc2ccc(C(O)Cc3cccnc3C)cc2)c2ccccc2)c(C)c1]

NaBH₄ (5 mg, 0.13 mmol) was added portion-wise to a solution of N-((2,4-dimethylphenyl)(phenyl)methyl)-2-(4-(2-(2-methylpyridin-3-yl)acetyl)phenyl)acetamide (20 mg, 0.04 mmol) in MeOH (5 mL) at 0° C. After the addition was complete, the mixture was allowed to warm up to rt and stirred overnight. The reaction was diluted with water (10 mL) and extracted with CH₂Cl₂ (3×10 mL). The combined extracts were dried over Na₂SO₄ and concentrated under reduced pressure. The resulting residue was purified by silica gel column chromatography (Petroleum ether/EtOAc=1/1) to obtain title compound (10 mg, yield: 49.8%). LCMS-A024: 465.7 [M+H]⁺; RT=1.34 min. ¹H NMR (DMSO-d₆, 400 MHz): δ 8.85 (d, J=8.4 Hz, 1H), 8.40 (dd, J1=1.2 Hz, J2=4.8 Hz, 1H), 7.45 (dd, J1=1.2 Hz, J2=7.2 Hz, 1H), 7.33-6.95 (m, 13H), 6.18 (d. J=8.4 Hz, 1H), 5.31 (d. J=4.4 Hz, 1H), 4.72 (t, J=2.8 Hz, 1H), 3.5 (s, 2H), 2.85 (t, J=5.2 Hz, 2H), 2.37 (s, 3H), 2.23 (s, 3H), 2.15 (s, 3H).

Following essential the same procedure as described in example 32, the compounds in table 5 were prepared.

TABLE 5

| | Name | NMR | LCMS |
|---|---|---|---|
| 33 | 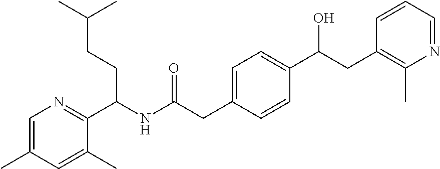 [SMILES: Cc1cnc(C(CCC(C)C)NC(=O)Cc2ccc(C(O)Cc3cccnc3C)cc2)c(C)c1] N-(l-(3,5-dimethylpyridin-2-yl)-4- methylpentyl)-2-(4-(1-hydroxy-2-(2- methylpyridin-3- yl)ethyl)phenyl)acetamide | ¹H NMR (MeOH-d₄, 400 MHz): δ 8.39 (d, J = 4.8 Hz, 1H), 8.15 (s, 1H), 7.44 (d, J = 7.6 Hz, 1H), 7.30-7.27 (m, 5H), 7.10-7.07 (m, 1H). 6.95-6.93 (m, 1H), 5.29-5.23 (m, 1H), 4.94-4.90 (m, 1H), 3.58 (s, 2H), 3.07-3.02 (m, 2H), 2.56 (s, 3H), 2.36 (s, 3H), 2.29 (s, 3H), 1.76-1.62 (m, 2H). 1.50-1.42 (m, 1H), 1.12- 1.06 (m, 2H), 0.81 (s, 3H), 0.79 (s, 3H). | LCMS- A012: 460.3 [M+H]+; Rt = 1.95 min. |

### Example 34

### 2-(4-(1-(2-amino-2-oxoethoxy)-2-(2-methylpyridin-3-yl)ethyl)phenyl)-N-((2,4-dimethylphenyl)(phenyl)methyl)acetamide

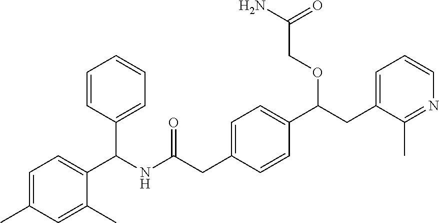 [SMILES: Cc1ccc(C(NC(=O)Cc2ccc(C(Cc3cccnc3C)OCC(N)=O)cc2)c2ccccc2)c(C)c1]

NaH (3.44 mg, 60% in oil) was added to a solution of N-((2,4-dimethylphenyl)(phenyl)methyl)-2-(4-(1-hydroxy-2-(2-methylpyridin-3-yl)ethyl)phenyl)acetamide (20 mg, 0.043 mmol) in THF (5 mL) at rt, and the reaction mixture was stirred for 30 min, followed by addition of 2-bromoacetamide (7.1 mg, 0.052 mmol). The mixture was stirred for 48 h, water (20 mL) was then added, and the mixture was extracted with ethyl acetate (20 mL×3). The combined organic layers were washed with brine, dried over sodium sulfate, and concentrated under reduced pressure to yield a residue which was purified by reverse phase HPLC using water/acetonitrile with 0.05% TFA to obtain title compound (3 mg, 13.6%). LCMS-A024: 522.3[M+H]⁺; Rt: 1.37 min. ¹H NMR (CDCl₃, 400 MHz): δ 8.59 (s, 1H), 7.79 (d, J=4.8 Hz, 1H), 7.39-7.03 (m, 9H), 6.92-6.79 (m, 4H), 6.29 (d, J=8.0 Hz, 1H), 6.03 (d, J=8.0 Hz, 1H), 5.49 (s, 1H), 4.53 (s, 1H), 3.80-3.66 (m, 1H), 3.55 (s, 2H), 3.21-3.03 (m, 2H), 2.60 (s, 3H), 2.22 (s, 3H), 2.15 (s, 3H).

### Example 35

### N-((4-chloro-2-methylphenyl)(phenyl)methyl)-2-(4-(2-(4,6-dihydroxypyrimidin-5-yl)ethoxy)phenyl)acetamide

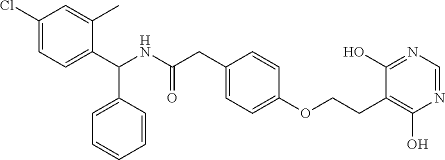 [SMILES: Cc1cc(Cl)ccc1C(NC(=O)Cc1ccc(OCCc2c(O)ncnc2O)cc1)c1ccccc1]

### (a) ethyl 2-(4-(2-bromoethoxy)phenyl)acetate

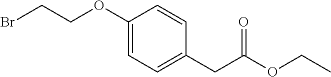 [SMILES: CCOC(=O)Cc1ccc(OCCBr)cc1]

Potassium carbonate (18.1 g, 130 mmol) and 1,2-dibromoethane (82.6 g, 439 mmol) were added to a stirred solution of ethyl 2-(4-hydroxyphenyl)acetate (13.6 g, 73 mmol) in CH₃CN (300 mL). The resulting mixture was stirred at 80° C. overnight. After cooling to rt, the mixture was extracted with EtOAc (100 mL×3). The combined extracts were washed with brine, dried over Na₂SO₄, and concentrated under reduced pressure. The obtained crude product was purified by flash chromatography (Petroleum ether/EtOAc=50/1) to give 16 g of ethyl 2-(4-(2-bromoethoxy)phenyl)acetate (yield 73%). LC-MS (024): 288.9 [M+H]⁺; RT=1.652 min. ¹H NMR (CDCl₃, 400 MHz): 7.22 (d, J=8.4 Hz, 2H), 6.88 (d, J=8.4 Hz, 2H), 4.29 (t, J=6 Hz, 2H), 4.16 (q, J=7.2 Hz, 2H), 3.64 (t, J=6.4 Hz, 2H), 1.26 (t, J=7.2 Hz, 3H).

### (b) diethyl 2-(2-(4-(2-ethoxy-2-oxoethyl)phenoxy)ethyl)malonate

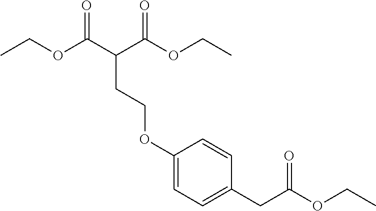 [SMILES: CCOC(=O)Cc1ccc(OCCC(C(=O)OCC)C(=O)OCC)cc1]

NaOEt (340 mg, 5 mmol) was added to a solution of diethyl malonate (800 mg, 5 mmol) in EtOH (5 mL). After the mixture was stirred at rt for 1 h, ethyl 2-(4-(2-bromoethoxy)phenyl)acetate (1.44 g, 5 mmol) was added. The resulting mixture was heated to reflux for 3 h. After cooling to rt, the solvent was evaporated under reduced pressure, and the residue was extracted with toluene (10 mL×3). The combined the extracts were concentrated under reduced pressure. The residue was purified by column chromatography to give the title compound as colorless oil (440 mg, yield 24%). LC-MS (010): 366.9 [M+H]⁺; RT=2.0 min.

### (c) ethyl 2-(4-(2-(4,6-dihydroxypyrimidin-5-yl)ethoxy)phenyl)acetate

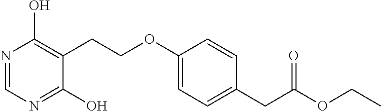 [SMILES: CCOC(=O)Cc1ccc(OCCc2c(O)ncnc2O)cc1]

Diethyl 2-(2-(4-(2-ethoxy-2-oxoethyl)phenoxy)ethyl)malonate (440 mg, 1.2 mmol) and formimidamide acetate (150 mg, 1.44 mmol) were added to a solution of NaOEt (98 mg, 1.44 mmol) in EtOH (5 mL). The resulting mixture was stirred at rt overnight, then neutralized with cone HCl at 0° C. After the organic solvent was concentrated under reduced pressure, the residue was washed with water. The precipitate was collected by filtration and dried under reduced pressure to afford the title compound as a white solid (70 mg, yield 18%). LC-MS (024): 319.0 [M+H]⁺; Rt=1.23 min.

### (d) 2-(4-(2-(4,6-dihydroxypyrimidin-5-yl)ethoxy)phenyl)acetic acid

This compound was synthesized from ethyl 2-(4-(2-(4,6-dihydroxypyrimidin-5-yl)ethoxy)phenyl)acetate essentially as example 15 (d) and was used directly to the next step without further purification. LC-MS (010): 290.9 [M+H]⁺; Rt=1.37 min.

### (e) N-((4-chloro-2-methylphenyl)(phenyl)methyl)-2-(4-(2-(4,6-dihydroxypyrimidin-5-yl)ethoxy)phenyl)acetamide

This compound was synthesized from 2-(4-(2-(4,6-dihydroxypyrimidin-5-yl)ethoxy)phenyl)acetic acid and (4-chloro-2-methylphenyl)(phenyl)methanamine hydrochloride essentially as example 2 (f) (6 mg, yield 14%). LC-MS (024): 504.0 [M+H]⁺; Rt=1.50 min. ¹H NMR (DMSO-d₆, 400 MHz): δ 11.93 (s, 2H), 8.88 (d, J=8 Hz, 1H), 7.97 (s 1H), 7.33 (t, d, J=8 Hz, 2H), 7.22-7.28 (m 3H), 7.16 (t, J=8.8 Hz, 4H), 7.10 (d, J=8.4 Hz, 1H), 6.86 (d, J=8.8 Hz, 1H), 6.17 (d, J=8 Hz, 1H), 3.93 (t, J=8 Hz, 2H), 3.43 (s, 2H), 2.72 (t, J=8 Hz, 2H), 2.18 (s, 3H).

### Example 36

### N-((4-chloro-2-methylphenyl)(phenyl)methyl)-2-(4-(2-(4-chloro-6-hydroxypyrimidin-5-yl)ethoxy)phenyl)acetamide

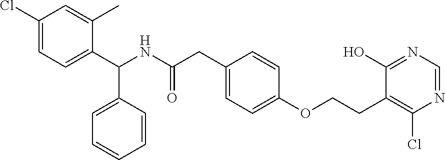 [SMILES: Cc1cc(Cl)ccc1C(NC(=O)Cc1ccc(OCCc2c(O)ncnc2Cl)cc1)c1ccccc1]

### (a) ethyl 2-(4-(2-(4,6-dichloropyrimidin-5-yl)ethoxy)phenyl)acetate

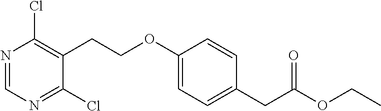 [SMILES: CCOC(=O)Cc1ccc(OCCc2c(Cl)ncnc2Cl)cc1]

ethyl 2-(4-(2-(4,6-dihydroxypyrimidin-5-yl)ethoxy)phenyl)acetate (318 mg, 1 mmol) was added to POCl₃ (3 mL), and the mixture was heated to reflux for 1 h. The excess POCl₃ was then evaporated under reduced pressure and the residue was poured into water, neutralized with NaHCO₃ (cone) at 0° C., then extracted with CH_Cl₂. The combined extracts were dried over Na₂SO₄, and concentrated under reduced pressure. The resulting residue was purified by flash chromatography to give the title compound as yellow oil (124 mg, yield 35%). LC-MS (024): 355.0 [M+H]⁺; Rt=1.71 min.

### (b) 2-(4-(2-(4-chloro-6-hydroxypyrimidin-5-yl)ethoxy)phenyl)acetic acid

This compound was synthesized from ethyl 2-(4-(2-(4,6-dichloropyrimidin-5-yl)ethoxy)phenyl)acetate essentially as example 15 (d) and was used directly to the next step without further purification. LC-MS (024): 309.0 [M+H]⁺; Rt=1.2 min.

### (c) N-((4-chloro-2-methylphenyl)(phenyl)methyl)-2-(4-(2-(4-chloro-6-hydroxypyrimidin-5-yl)ethoxy)phenyl)acetamide

This compound was synthesized from 2-(4-(2-(4-chloro-6-hydroxypyrimidin-5-yl)ethoxy)phenyl)acetic acid and (4-chloro-2-methylphenyl)(phenyl)methanamine hydrochloride essentially as example 2 (1) (8 mg, yield 24%). LC-MS (022): 522.0 [M+H]⁺; Rt=1.7 min. ¹H NMR (DMSO-d₆, 400 MHz): δ 12.87 (s, 1H), 8.96 (d, J=8 Hz, 1H), 8.19 (s 1H), 7.40 (t, d, J=7.6 Hz, 2H), 7.29-7.35 (m 3H), 7.21-7.24 (m, 4H), 7.16 (d, J=8.4 Hz, 1H), 6.91 (d, J=8.4 Hz, 1H), 6.24 (d, J=8 Hz, 1H), 4.13 (t, J=8 Hz, 2H), 3.50 (s, 2H), 3.00 (t, J=7.2 Hz, 2H), 2.25 (s, 3H).

### Example 37

### N-((4-chloro-2-methylphenyl)(phenyl)methyl)-2-(4-(2-(pyrimidin-5-yl)ethoxy)phenyl)acetamide

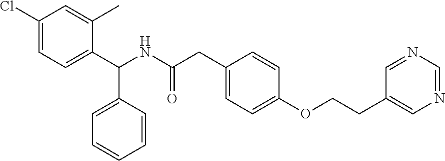 [SMILES: Cc1cc(Cl)ccc1C(NC(=O)Cc1ccc(OCCc2cncnc2)cc1)c1ccccc1]

### (a) ethyl 2-(4-(2-(pyrimidin-5-yl)ethoxy)phenyl)acetate

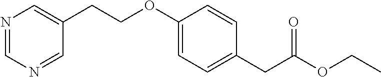 [SMILES: CCOC(=O)Cc1ccc(OCCc2cncnc2)cc1]

Pd/C (5 mg) and triethylamine (30 mg, 0.297 mmol) were added to a solution of ethyl 2-(4-(2-(4,6-dichloropyrimidin-5-yl)ethoxy)phenyl)acetate (48 mg, 0.135 mmol) in EtOH (2 mL). The mixture was stirred under hydrogen (1 atm) for 2 h. The Pd/C was removed by filtration through a pad of Celite. Concentration of organic solvent under reduced pressure gave the title compound as a white solid (35 mg), which was used directly in the next step without further purification. LC-MS (024): 287.0 [M+H]⁺; Rt=1.42 min

### (b) 2-(4-(2-(pyrimidin-5-yl)ethoxy)phenyl)acetic acid

This compound was synthesized from ethyl 2-(4-(2-(pyrimidin-5-yl)ethoxy)phenyl)acetate essentially as example 15 (d) and was used directly to the next step without further purification. LC-MS (024): 259.0 [M+H]⁺; RT=1.18 min

### (c) N-((4-chloro-2-methylphenyl)(phenyl)methyl)-2-(4-(2-(pyrimidin-5-yl)ethoxy)phenyl)acetamide

HATU (41 mg, 0.11 mmol) was added to a solution of 2-(4-(2-(pyrimidin-5-yl)ethoxy)phenyl)acetic acid (28 mg, 0.11 mmol) in DMF (2 mL) and the mixture was stirred at rt for 40 min. Then (4-chloro-2-methylphenyl)(phenyl)methanamine hydrochloride (40 mg, 0.09 mmol) was added, and the resulting mixture was stirred at rt for 3 h. The reaction mixture was poured into water and extracted with EtOAc (10 mL×3). The combined extracts were washed with water, and brine, and then dried over Na₂SO₄. After removal of solvent under reduced pressure, the residue was used purified by prep-TLC to give the title compound as a white solid (16 mg, yield 31%). LC-MS (024): 472.0 [M+H]⁺; RT=1.7 min. ¹H NMR (DMSO-d₆, 400 MHz): δ 9.12 (s, 1H), 8.95 (d, J=8 Hz, 1H), 8.84 (s 2H), 7.39 (t, d, J=7.6 Hz, 2H), 7.28-7.34 (m 3H), 7.21-7.23 (m, 4H), 7.15 (d, J=8 Hz, 1H), 6.91 (d, J=8.8 Hz, 1H), 6.23 (d, J=8.4 Hz, 1H), 4.26 (t, J=6.4 Hz, 2H), 3.49 (s, 2H), 3.11 (t, J=6.4 Hz, 2H), 2.24 (s, 3H).

### Example 38

### N-((4-chloro-2-methylphenyl)(phenyl)methyl)-2-(2-(((2-methylpyridin-3-yl)methyl)amino)thiazol-4-yl)acetamide

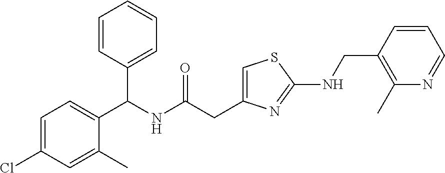 [SMILES: Cc1cc(Cl)ccc1C(NC(=O)Cc1csc(NCc2cccnc2C)n1)c1ccccc1]

### (a) 2-Methylnicotinaldehyde

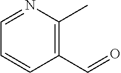

(2-Methyl-pyridin-3-yl)-methanol (1.0 g, 8.13 mmol) was dissolved in dry CH₂Cl₁ (30 mL) and argon gas was purged for 10 min. Dess-Martin periodinane (4.5 g, 10.5 mmol) was added to the reaction mixture at 0° C. The reaction mixture was allowed to come to rt and further stirred for 2 h. The reaction mixture was quenched with saturated NaHCO₃ solution. The organic product was extracted with EtOAc. The combined organic extracts were washed with saturated sodium thiosulfate solution, water and brine solution. Solvent was removed under reduced pressure to get the crude 2-methylnicotinaldehyde (600 mg, crude). which was used as such for the next step. TLC: 100% EtOAc, R_{f}=0.4. ¹H NMR (300 MHz, CDCl₃) δ 10.33 (s, 1H), 8.70-8.68 (dd, J=4.8 Hz, 1.8 Hz, 1H), 8.12-8.09 (dd, J=7.7 Hz, 1.8 Hz, 1H), 7.36-7.32 (dd, J=7.7 Hz, 4.8 Hz, 1H), 2.89 (s, 3H)

### (b) methyl 2-(2-(((2-methylpyridin-3-yl)methyl)amino)thiazol-4-yl)acetate

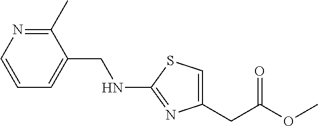 [SMILES: COC(=O)Cc1csc(NCc2cccnc2C)n1]

Methyl-2-amino-4-thiazolacetate (420 mg, 2.5 mmol) was added to a solution of 2-methylnicotinaldehyde (300 mg, 2.5 mmol) in 1,2-dichloroethane (5 mL), followed by sodium triacetoxyborohydride (1.0 g, 5.0 mmol) at 0. The reaction mixture was allowed to come to rt and stirred for 10 h. The reaction mixture was quenched with 10% aqueous NaHCO₃ solution and extracted with EtOAc. The combined extracts were dried over anhydrous sodium sulfate. Solvent was removed under reduced pressure and the crude product was purified by column chromatography (silica gel 60-120 mesh, eluent 5-10% MeOH in CH₂Cl₂) to afford methyl 2-(2-(((2-methylpyridin-3-yl)methyl)amino)thiazol-4-yl)acetate (80 mg) along with (2-methyl-pyridin-3-yl)-methanol as mixture. TLC: 100% EtOAc, R_{f}=0.3. LC-MS purity>40%

### (c) 2-(2-(((2-Methylpyridin-3-yl)methyl)amino)thiazol-4-yl)acetic acid

This compound was synthesized from methyl 2-(2-(((2-methylpyridin-3-yl)methyl)amino)thiazol-4-yl)acetate essentially as example 15 (d) and was used directly to the next step without further purification. TLC: 100% EtOAc, R_{f}=0.2. LC-MS purity>42%

### (d) N-((4-chloro-2-methylphenyl)(phenyl)methyl)-2-(2-(((2-methylpyridin-3-yl)methyl)amino)thiazol-4-yl)acetamide

This compound was synthesized from 2-(2-(((2-methylpyridin-3-yl)methyl)amino)thiazol-4-yl)acetic acid and (4-chloro-2-methylphenyl)(phenyl)methanamine essentially as example 7 (b) (25 mg, yield 15%). TLC: petroleum ether:EtOAc 3:7, R_{f}=0.3. LC-MS purity>98%, HPLC purity>97%. ¹H NMR (400 MHz, MeOD) δ 8.31 (m, 1H), 7.73-7.71 (d, J=7.5 Hz, 1H), 7.33-7.26 (m, 2H), 7.22-7.11 (m, 6H), 7.07-7.05 (m, 1H), 6.36 (br s, 1H), 6.28 (s, 1H), 4.46 (s, 2H), 3.51 (s, 2H), 2.51 (s, 3H), 2.22 (s, 3H).

### Example 39

### N-((4-chloro-2-methylphenyl)(phenyl)methyl)-2-(3-methyl-4-((2-methylpyridin-3-yl)methoxy)phenyl)acetamide

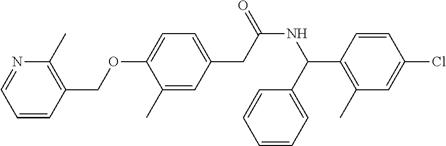 [SMILES: Cc1cc(CC(=O)NC(c2ccccc2)c2ccc(Cl)cc2C)ccc1OCc1cccnc1C]

### (a) N-((4-chloro-2-methylphenyl)(phenyl)methyl)-2-(4-methoxy-3-methylphenyl)acetamide

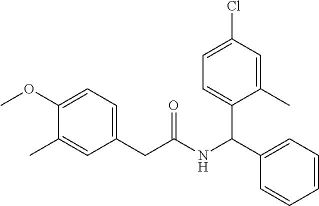 [SMILES: COc1ccc(CC(=O)NC(c2ccccc2)c2ccc(Cl)cc2C)cc1C]

This compound was synthesized from 2-(4-methoxy-3-methylphenyl)acetic acid and (4-chloro-2-methylphenyl)(phenyl)methanamine essentially as described in example 2 (f) and was used in the next step without any further purification. LC-MS: 394 [M+H]⁺; Rt: 1.818 min.

### (b) N-((4-chloro-2-methylphenyl)(phenyl)methyl)-2-(4-hydroxy-3-methylphenyl)acetamide

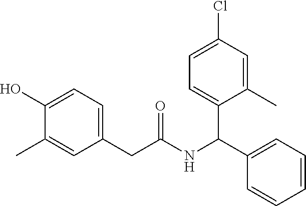 [SMILES: Cc1cc(CC(=O)NC(c2ccccc2)c2ccc(Cl)cc2C)ccc1O]

BBr₃ (76 mg, 0.31 mmol) was added to a solution of N-((4-chloro-2-methylphenyl)(phenyl)methyl)-2-(4-methoxy-3-methylphenyl)acetamide (100 mg, 0.25 mmol) in 10 mL CH₂Cl₂ under N₂ atmosphere at −78° C. The mixture was allowed to warm up to rt overnight. The solvent was evaporated under reduced pressure to get N-((4-chloro-2-methylphenyl)(phenyl)methyl)-2-(4-hydroxy-3-methylphenyl)acetamide (86 mg, yield 89%) as yellow solid, which was used in the next step without any further purification. LC-MS: 380 [M+H]⁺; Rt: 1.658 min.

### (c) N-((4-chloro-2-methylphenyl)(phenyl)methyl)-2-(3-methyl-4-((2-methylpyridin-3-yl)methoxy)phenyl)acetamide

3-(Chloromethyl)-2-methylpyridine (49 mg, 0.27 mmol) and K₂CO₃ (91 mg, 0.66 mmol) were added to a solution of N-((4-chloro-2-methylphenyl)(phenyl)methyl)-2-(4-hydroxy-3-methylphenyl)acetamide (86 mg, 0.22 mmol) in 20 mL of CH₃CN. The resulting mixture was stirred at 50° C. overnight. After cooling to rt, the solid was filtered off. The organic solvent was removed under reduced pressure and the residue was purified by Pre-TLC (Petroleum ether/EtOAc=3/2) to obtain N-((4-chloro-2-methylphenyl)(phenyl)methyl)-2-(3-methyl-4-((2-methylpyridin-3-yl)methoxy)phenyl)acetamide (60 mg, yield: 54%) as white solid. LC-MS: 485 [M+H]⁺; Rt: 1.510 min. ¹H NMR (DMSO-d₆, 400 MHz): δ 8.91 (d, J=8.0 Hz, 1H), 8.41-8.40 (m, 1H), 7.80-7.78 (m, 1H), 7.35-6.98 (m, 12H), 6.18 (d, J=8.5 Hz, 1H), 5.11 (s, 2H), 3.42 (s, 2H), 2.51 (s, 3H), 2.19 (s, 3H), 2.15 (s, 3H).

### Example 40

### N-(1-(4-chlorophenyl)-2-methylpropyl)-2-(4-((2-methylpyridin-3-yl)methoxy)phenyl)acetamide

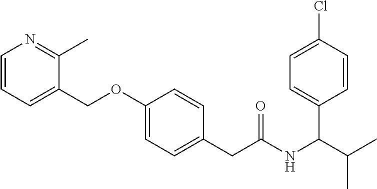 [SMILES: Cc1ncccc1COc1ccc(CC(=O)NC(c2ccc(Cl)cc2)C(C)C)cc1]

### (a) 1-(4-chlorophenyl)-2-methylpropan-1-amine

i-PrMgBr (2N, 3 mL, 6 mmol) was added drop-wise to a solution of 4-chlorobenzonitrile (680 mg, 5 mmol) in THF (dry, 5 mL) at r.t under argon atmosphere. After the addition, the mixture was stirred at rt overnight. Methanol (5 mL) was then added, followed by NaBH₄ (222 mg, 6 mmol). After stirring for additional 30 min, the mixture was quenched with water, and then extracted with EtOAc. The combined organic layers were extracted with HCl (1N). The pH value of aqueous phase was adjusted the pH to 9˜10 with NaOH (1N), then extracted with EtOAc again. The combined organic layers were washed with brine, and dried over Na₂SO₄. After removal of the solvent, the residue was solidified by using HCl/Et₂O. The solid was collected by filtration to give the titled compound as a yellow solid (200 mg, yield 18%). LC-MS: m/z=182, (m+H)⁺

### (b) N-(1-(4-chlorophenyl)-2-methylpropyl)-2-(4-((2-methylpyridin-3-yl)methoxy)phenyl)acetamide

This compound was synthesized from 2-(4-((2-methylpyridin-3-yl)methoxy)phenyl)acetic acid and 1-(4-chlorophenyl)-2-methylpropan-1-amine essentially as example 15 (e) (20 mg, yield 23%). LC-MS: 423[M+H]⁺. ¹H NMR (DMSO, 400 MHz): δ 8.41-8.39 (m, 2H), 7.77-7.74 (m, 1H), 7.35 (d, J=8.0 Hz, 2H), 7.28 (d, J=8.0 Hz, 2H), 7.23 (dd, J=4.8 Hz, 8.0 Hz, 1H), 7.16 (d, J=8.0 Hz, 2H), 6.95 (d, J=8.8 Hz, 2H), 5.09 (s, 2H), 4.51 (t, J=8.8 Hz, 1H), 3.44-3.34 (m, 2H), 1.95-1.87 (m, 1H), 0.86 (d, J=6.8 Hz, 3H), 0.69 (s, J=6.8 Hz, 3H).

### Example 41

### 2-(4-((2-methylpyridin-3-yl)methoxy)phenyl)-N-(4-(p-tolyl)tetrahydro-2H-pyran-4-yl)acetamide

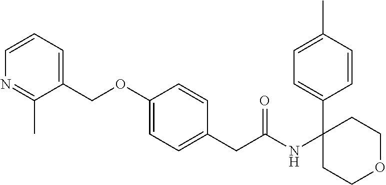 [SMILES: Cc1ccc(C2(NC(=O)Cc3ccc(OCc4cccnc4C)cc3)CCOCC2)cc1]

### (a) 2-(4-((2-methylpyridin-3-yl)methoxy)phenyl)-N-(4-(p-tolyl)tetrahydro-2H-pyran-4-yl)acetamide

This compound was synthesized from 2-(4-((2-methylpyridin-3-yl)methoxy)phenyl)acetic acid and 4-(4-chlorophenyl)tetrahydro-2H-pyran-4-amine essentially as described in example 2 (f) (41 mg, yield: 60%). LC-MS: 451 [M+H]⁺; RT=1.532 min. ¹H NMR (DMSO-d₆, 400 MHz): δ 8.40-8.38 (m, 1H), 8.25 (s, 1H), 7.77-7.75 (m, 1H), 7.34-7.29 (m, 4H), 7.24-7.18 (m, 3H), 6.97 (d, J=8.8 Hz, 2H), 5.09 (s, 2H), 3.69-3.66 (m, 2H), 3.53 (t, J=11.2 Hz, 2H), 3.41 (s, 2H), 2.48 (s, 3H), 2.23 (d, J=13.2 Hz, 2H), 1.87-1.81 (m, 2H).

### Example 42

### N-((2,4-dimethylphenyl)(thiazol-5-yl)methyl)-2-(4-((2-methylpyridin-3-yl)methoxy)phenyl)acetamide

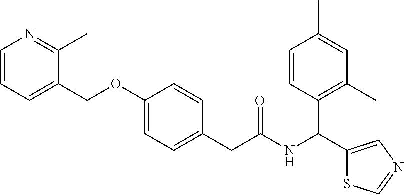 [SMILES: Cc1ccc(C(NC(=O)Cc2ccc(OCc3cccnc3C)cc2)c2cncs2)c(C)c1]

### (a) (2,4-dimethylphenyl)(thiazol-5-yl)methanamine

i-PrMgCl (3.6 mL, 7.2 mmol, 2N solution in THF) was added to a stirred solution of 5-bromothiazole (972 mg, 6 mmol) in THF (20 mL) at 0° C. under nitrogen atmosphere. After stirring at 0° C. for 30 min, 2,4-dimethylbenzonitrile (943 mg, 7.2 mmol) was added. The solution was further stirred for 1 h at 0° C., then NaBH₄ (456 mg, 12 mmol) was added, followed by addition of MeOH (20 mL). The resulting mixture was allowed to stir at rt overnight, and then NH₄Cl (sat.) was added. The resulting mixture was extracted with EtOAc, the combined extracts were washed with brine, dried over sodium sulfate and concentrated under reduced pressure. The crude product was purified by flash column (petroleum ether/EtOAc=1/1 to CH₂Cl₂/MeOH=50/1) to yield (2,4-dimethylphenyl)(thiazol-5-yl)methanamine (50 mg, yield: 5%). LC-MS (024): m/z=202.0, [M−NH2]⁺. Rt=1.17 min, purity 96.5%

### (b) N-((2,4-dimethylphenyl)(thiazol-5-yl)methyl)-2-(4-((2-methylpyridin-3-yl)methoxy)phenyl)acetamide

This compound was synthesized from 2-(4-((2-methylpyridin-3-yl)methoxy)phenyl)acetic acid and (2,4-dimethylphenyl)(thiazol-5-yl)methanamine essentially as described in example 2 (f) (12 mg, Yield: 26.3%). LC-MS: m/z=458.2, [M+H]⁺. Rt=1.89 min, purity 95.62%. ¹H NMR (MEOD, 400 MHz): δ 8.38 (m, 1H), 7.88 (m, 1H), 7.76 (m, 1H), 7.54 (d, J=3.6 Hz, 1H), 7.28 (m, 3H), 6.98 (m, 5H), 6.57 (s, 1H), 5.12 (s, 2H), 3.56 (s, 3H), 2.58 (s, 3H), 2.28 (m, 3H).

### Example 43

### N-((5-methylpyridin-2-yl)(phenyl)methyl)-2-(4-((2-methylpyridin-3-yl)methoxy)phenyl)acetamide

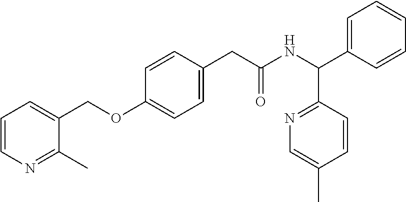 [SMILES: Cc1ccc(C(NC(=O)Cc2ccc(OCc3cccnc3C)cc2)c2ccccc2)nc1]

### (a) N-((5-methylpyridin-2-yl)(phenyl)methyl)-2-(4-((2-methylpyridin-3-yl)methoxy)phenyl)acetamide

This compound was synthesized from 2-(4-((2-methylpyridin-3-yl)methoxy)phenyl)acetic acid and (5-methylpyridin-2-yl)(phenyl)methanamine essentially as described in example 2 (f) (26 mg, Yield: 52.6%). LC-MS: m/z=438.2, [M+H]⁺. Rt=1.87 min. purity 91.95%. ¹H NMR (MEOD, 400 MHz): δ 8.37 (m, 2H), 7.88 (m, 1H), 7.58 (m, 1H), 7.21-7.31 (m, 9H), 6.99 (m, 2H), 6.13 (s, 1H), 5.50 (s, 2H), 3.56 (s, 2H), 3.58 (s, 2H), 2.58 (m, 3H), 2.34 (s, 3H).

### Example 44

### N-(1-(2,4-dimethylphenyl)-4-methylpentyl)-2-(4-(1-hydroxy-2-(2-methylpyridin-3-yl)ethyl)phenyl)acetamide

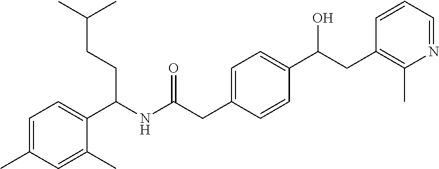 [SMILES: Cc1ccc(C(CCC(C)C)NC(=O)Cc2ccc(C(O)Cc3cccnc3C)cc2)c(C)c1]

### (a) N-(1-(2,4-dimethylphenyl)-4-methylpentyl)-2-(4-(2-(2-methylpyridin-3-yl)acetyl)phenyl)acetamide

This compound was synthesized from 2-(4-(2-(2-methylpyridin-3-yl)acetyl)phenyl)acetic acid and 1-(2,4-dimethylphenyl)-4-methylpentan-1-amine essentially as described in example 2 (f) (48 mg, Yield: 32%). LC-MS (A012): m/z=457.2, [M+H]⁺. Rt=1.76 min

### (b) N-(1-(2,4-dimethylphenyl)-4-methylpentyl)-2-(4-(1-hydroxy-2-(2-methylpyridin-3-yl)ethyl)phenyl)acetamide

This compound was synthesized from N-(1-(2,4-dimethylphenyl)-4-methylpentyl)-2-(4-(2-(2-methylpyridin-3-yl)acetyl)phenyl)acetamide essentially as described in example 5 (a) (5 mg, yield: 11%). LC-MS (A024): 459.3 [M+H]⁺; RT=1.44 min. ¹H NMR (MEOD, 400 MHz): δ 8.21 (d, J=4.8 Hz, 1.2 Hz, 1H), 7.49-7.46 (m, 1H), 7.24-7.19 (m, 4H), 7.16-7.10 (m, 2H), 6.98-6.94 (m, 2H), 5.01 (t, 1H), 4.87 (t, 1H), 3.48 (d, J=3.2 Hz, 2H), 3.07-2.98 (m, 2H), 2.41 (s, 3H), 2.30 (s, 3H), 2.26 (s, 3H), 1.72-1.69 (m, 2H), 1.56-1.53 (m, 1H), 1.30-1.10 (m, 2H), 0.87 (t, 6H).

### Example 45

### N-(1-(3,5-dimethylpyridin-2-yl)-4-methylpentyl)-2-(4-((2-methylpyridin-3-yl)methoxy)phenyl)acetamide

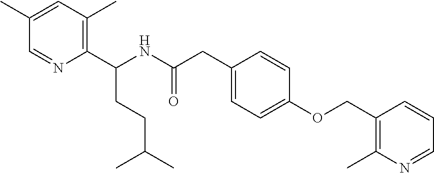 [SMILES: Cc1cnc(C(CCC(C)C)NC(=O)Cc2ccc(OCc3cccnc3C)cc2)c(C)c1]

### (a) 1-(3,5-dimethylpyridin-2-yl)-4-methylpentan-1-amine

To a solution of 3,5-dimethylpicolinonitrile (792 mg, 6 mmol) in 20 mL of THF under N₂ protection at 0° C. was dropped isopentylmagnesium bromide which was freshly prepared and used immediately (0.5N in THF, 21.6 mL). After the addition, the mixture was allowed to warm up to rt and stirred for 2 h. Then NaBH₄ (456 mg, 12 mmol) was added followed by addition of MeOH (15 mL). After stirring for 3 h, 15 mL of NH₄Cl (sat, 15 mL) aqueous solution was added to quench the reaction, and the mixture was extracted with EtOAc (20 mL×4). The combined EtOAc extracts were extracted with 1N HCl (15 mL×3). The pH value of the aqueous phase was adjusted to ˜11, and then EtOAc (20 mL×3) was used to extract the desired amine. The combined EtOAc extracts were dried over Na₂SO₄. Removal of solvent under reduced pressure yielded crude 1-(3,5-dimethylpyridin-2-yl)-4-methylpentan-1-amine (800 mg) as an amber oil, which was used directly with any further purification. LCMSA024: 207.2 [M+H]⁺: Rt=1.29 min; Purity=100% (254 nm).

### (b) N-(1-(3,5-dimethylpyridin-2-yl)-4-methylpentyl)-2-(4-((2-methylpyridin-3-yl)methoxy)phenyl)acetamide

This compound was synthesized from 1-(3,5-dimethylpyridin-2-yl)-4-methylpentan-1-amine and 12-(4-((2-methylpyridin-3-yl)methoxy)phenyl)acetic acid essentially as described in example 2 (f) (33.6 mg, Yield: 49.2%). LC-MS (024): 46.3[M+H]⁺; Rt: 1.23 min. ¹H NMR (DMSO, 400 MHz): δ 8.59 (br, 1H), 8.31-8.23 (m, 2H), 7.71-7.68 (m, 2H), 7.15 (d, J=8.4 Hz, 2H), 6.95 (d, J=8.4 Hz, 2H), 5.18 (s, 2H), 4.92 (t, J=7.2 Hz, 1H), 3.43 (d, J=13.6 Hz, 1H), 3.34 (d, J=14 Hz, 1H), 2.64 (s, 3H), 2.31 (s, 3H), 2.28 (s, 3H), 1.24 (m, 2H), 1.46-1.40 (m, 1H), 1.19-0.95 (m, 2H), 0.77 (d, J=6.8 Hz, 3H), 0.76 (d, J=7.2 Hz, 3H).

### Example 46

### 2-(1-(4-(2-(((2,4-dimethylphenyl)(phenyl)methyl)amino)-2-oxoethyl)phenyl)-2-(2-methylpyridin-3-yl)ethoxy)acetic acid

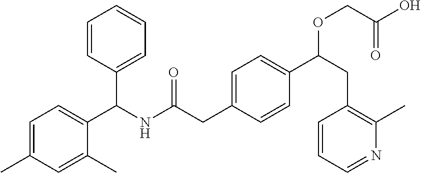 [SMILES: Cc1ccc(C(NC(=O)Cc2ccc(C(Cc3cccnc3C)OCC(=O)O)cc2)c2ccccc2)c(C)c1]

### (a) N-((2,4-dimethylphenyl)(phenyl)methyl)-2-(4-(2-(2-methylpyridin-3-yl)acetyl)phenyl)acetamide

This compound was synthesized from (2,4-dimethylphenyl)(phenyl)methanamine and 2-(4-(2-(2-methylpyridin-3-yl)acetyl)phenyl)acetic acid essentially as described in example 2 (f) (490 mg, Yield: 35%). LC-MS (A026): m/z=463.2, [M+H]⁺. Rt=1.98 min

### (b) N-((2,4-dimethylphenyl)(phenyl)methyl)-2-(4-(1-hydroxy-2-(2-methylpyridin-3-yl)ethyl)phenyl)acetamide

This compound was synthesized from N-((2,4-dimethylphenyl)(phenyl)methyl)-2-(4-(2-(2-methylpyridin-3-yl)acetyl)phenyl)acetamide essentially as described in example 5 (a) (500 mg, yield: 90%). LC-MS (A012): 465.2 [M+H]⁺; RT=1.60 min.

### (c) Ethyl 2-(1-(4-(2-(((2,4-dimethylphenyl)(phenyl)methyl)amino)-2-oxoethyl)phenyl)-2-(2-methylpyridin-3-yl)ethoxy)acetate

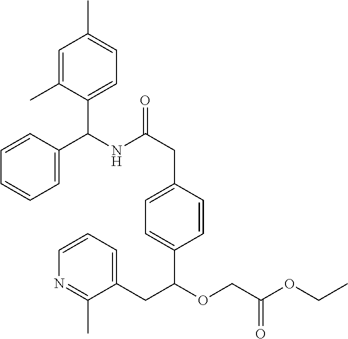 [SMILES: CCOC(=O)COC(Cc1cccnc1C)c1ccc(CC(=O)NC(c2ccccc2)c2ccc(C)cc2C)cc1]

Ethyl 2-bromoacetate (112 mg, 0.67 mmol), Cs₂CO₃ (241 mg, 0.74 mmol), and KI (1.2 mg, 0.007 mmol) were added to a solution of N-((2,4-dimethylphenyl)(phenyl)methyl)-2-(4-(1-hydroxy-2-(2-methylpyridin-3-yl)ethyl)phenyl)acetamide (170 mg, 0.37 mmol) in MeCN (20 mL). After the addition, the mixture was heated at 65° C. overnight. The reaction was diluted with water (30 mL), and extracted with EtOAc (50 mL×3), dried over Na₂SO₄, and concentrated under reduced pressure. The resulting residue was purified by Pre-TLC to obtain ethyl 2-(1-(4-(2-(((2.4-dimethylphenyl)(phenyl)methyl)amino)-2-oxoethyl)phenyl)-2-(2-methylpyridin-3-yl)ethoxy)acetate (168 mg, yield: 100%). LC-MS (A024): 551 [M+H]⁺; RT=1.43 min.

### (d) 2-(1-(4-(2-(((2,4-dimethylphenyl)(phenyl)methyl)amino)-2-oxoethyl)phenyl)-2-(2-methylpyridin-3-yl)ethoxy)acetic acid

This compound was synthesized from ethyl 2-(1-(4-(2-(((2,4-dimethylphenyl)(phenyl)methyl)amino)-2-oxoethyl)phenyl)-2-(2-methylpyridin-3-yl)ethoxy)acetate essentially as described in example 15 (d) (9 mg, yield: 8%). LC-MS (A026): 523.2 [M+H]⁺; RT=1.74 min. ¹H NMR (DMSO, 400 MHz): δ 8.87 (d, J=8.4 Hz, 1H), 8.67 (d, J=5.6 Hz, 1H), 8.17 (d, J=7.6 Hz, 1H), 7.76 (t, 1H), 7.33-7.16 (m, 9H), 7.00-6.98 (m, 3H), 6.18 (d, J=8.4 Hz, 1H), 5.48 (d, J=4.8 Hz, 1H), 4.85 (s, 2H), 3.50 (s, 2H), 3.10-3.07 (m, 2H) 2.53 (s, 3H), 2.22 (s, 3H), 2.16 (s, 3H).

### Example 47

### N-((4-hydroxy-2-methylphenyl)(phenyl)methyl)-2-(4-((2-methylpyridin-3-yl)methoxy)phenyl)acetamide

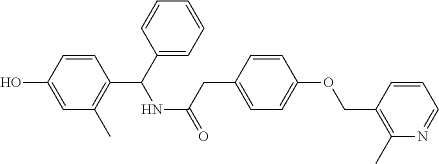 [SMILES: Cc1cc(O)ccc1C(NC(=O)Cc1ccc(OCc2cccnc2C)cc1)c1ccccc1]

### (a) 4-hydroxy-2-methylbenzonitrile

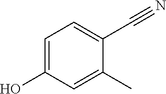

Decanethiol (261 mg, 1.5 mmol) and t-BuOK (168 mg, 1.5 mmol) were added to a solution of 4-methoxy-2-methylbenzonitrile (147 mg, 1 mmol) in DMF (5 mL). The reaction mixture was stirred at 110° C. for 3 h. The mixture was then diluted with water (30 mL) and extracted with EtOAc (10 mL×3). The extracts were washed with brine (10 mL×3), dried over Na₂SO₄, and concentrated under reduced pressure. The resulting residue was purified by silica gel column with (petroleum ether:EtOAc=10:1) to provide 4-hydroxy-2-methylbenzonitrile (70 mg, yield: 52.6%). LC-MS (011): 134.70 [M+H]⁺; Rt: 1.44 min, Purity: 80% (254 nm).

### (b) 4-(amino(phenyl)methyl)-3-methylphenol

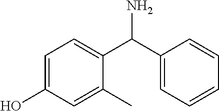 [SMILES: Cc1cc(O)ccc1C(N)c1ccccc1]

This compound was synthesized from 4-hydroxy-2-methylbenzonitrile and phenylmagnesium bromide essentially as described in example 1 (a) (150 mg, yield: 28.8%). LCMSA024: MS: 197 [M-NH₂]⁺; Rt=1.11 min.

### (c) (4-(benzyloxy)-2-methylphenyl)(phenyl)methanamine

This compound was synthesized from 4-(benzyloxy)-2-methylbenzonitrile and phenylmagnesium bromide essentially as described in example 1 (a) (400 mg, yield: 37%). LC-MS (010): 288.7 [M-NH₂]⁺; Rt=1.593 min, purity: 100% 214.

### (e) N-((4-hydroxy-2-methylphenyl)(phenyl)methyl)-2-(4-((2-methylpyridin-3-yl)methoxy)phenyl)acetamide

This compound was synthesized from 4-(amino(phenyl)methyl)-3-methylphenol and 2-(4-((2-methylpyridin-3-yl)methoxy)phenyl)acetic acid essentially as described in example 15 (e) (7 mg, yield: 8%). LC-MS039: 453.3[M+H]⁺; RT=1.566 min; Purity: 100% (214 nm). ¹H NMR (MeOD, 400 MHz): d 8.38 (d, J=4.0 Hz, 1H), 7.88 (d, J=7.6 Hz, 1H), 7.32-7.28 (m, 6H), 7.16 (d, J=7.2 Hz, 2H), 6.99 (d, J=8.4 Hz, 2H), 6.84 (d, J=8.4 Hz, 1H), 6.63 (d, J=2.0 Hz, 1H), 6.55 (dd, J=8.4 Hz, 2.4 Hz, 1H), 6.25 (s, 1H), 5.13 (s, 2H), 3.54 (s, 2H), 2.59 (s, 3H), 2.16 (s, 3H).

### Example 48

### 2-(4-((4-chlorobenzyl)oxy)phenyl)-N-((4-chlorophenyl)(phenyl)methyl)acetamide

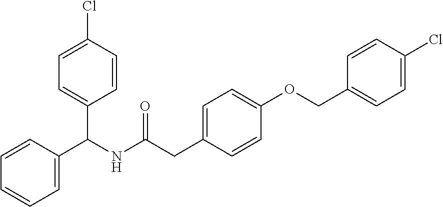 [SMILES: O=C(Cc1ccc(OCc2ccc(Cl)cc2)cc1)NC(c1ccccc1)c1ccc(Cl)cc1]

This compound was synthesized from N-((4-chlorophenyl)(phenyl)methyl)-2-(4-hydroxyphenyl)acetamide and 1-(bromomethyl)-4-chlorobenzene essentially as described in example 12 (c) (0.025 g, 1.85%) ¹H NMR (400 MHz, DMSO) δ 8.99-9.02 (d, 1H), 7.45-7.48 (m, 3H), 7.37-7.39 (d, 2H), 7.30-7.34 (m, 2H), 7.23-7.28 (m, 5H), 7.16-7.19 (d, 2H), 6.90-6.93 (d, 2H), 6.06-6.09 (d, 1H), 5.07 (s, 2H), 3.46 (s, 2H), MS (ESI+) m/z 474.1 (M−H); LCMS purity 95.01%.

### Biological Data

As stated above, the compounds according to Formula (I) are RORγ modulators, and are useful in the treatment of diseases mediated by RORγ. The biological activities of the compounds according to Formula (I) can be determined using any suitable assay for determining the activity of a candidate compound as a RORγ modulator, as well as tissue and in vivo models.

### Dual Fluorescence Energy Transfer (FRET) Assay

This assay is based on the knowledge that nuclear receptors interact with cofactors (transcription factors) in a ligand dependent manner. RORγ is a typical nuclear receptor in that it has an AF2 domain in the ligand binding domain (LBD) which interacts with co-activators. The sites of interaction have been mapped to the LXXLL motifs in the co-activator SRC1(2) sequences. Short peptide sequences containing the LXXLL motif mimic the behavior of full-length co-activator.

The assay measures ligand-mediated interaction of the co-activator peptide with the purified bacterial-expressed RORγ ligand binding domain (RORγ-LBD) to indirectly assess ligand binding. RORγ has a basal level of interaction with the co-activator SRC1(2) in the absence of ligand, thus it is possible to find ligands that inhibit or enhance the RORγ/SRC1(2) interaction.

### Materials

### Generation of RORγ-LBD Bacterial Expression Plasmid

Human RORγ Ligand Binding Domain (RORγ-LBD) was expressed in E. coli strain BL21(DE3) as an amino-terminal polyhistidine tagged fusion protein. DNA encoding this recombinant protein was sub-cloned into a modified pET21 a expression vector (Novagen). A modified polyhistidine tag (MKKHHHHHHLVPRGS) (SEQ ID No: 1) was fused in frame to residues 263-518 of the human RORγ sequence.

### Protein Purification

Approximately 50 g E. coli cell pellet was resuspended in 300 mL of lysis buffer (30 mM imidazole pH 7.0 and 150 mM NaCl). Cells were lysed by sonication and cell debris was removed by centrifugation for 30 minutes at 20,000 g at 4° C. The cleared supernatant was filtered through a 0.45 μM cellulose acetate membrane filter. The clarified lysate was loaded onto a column (XK-26) packed with ProBond Nickel Chelating resin (InVitrogen), pre-equilibrated with 30 mM imidazole pH 7.0 and 150 mM NaCl. After washing to baseline absorbance with the equilibration buffer, the column was developed with a gradient from 30 to 500 mM imidazole pH 7.0. Column fractions containing the RORγ-LBD protein were pooled and concentrated to a volume of 5 mL. The concentrated protein was loaded onto a Superdex 200 column pre-equilibrated with 20 mM Tris-Cl pH 7.2 and 200 mM NaCl. The fractions containing the desired RORγ-LBD protein were pooled together.

### Protein Biotinylation

Purified RORγ-LBD was buffer exchanged by exhaustive dialysis [3 changes of at least 20 volumes (>8000×)]against PBS [100 mM NaPhosphate, pH 8 and 150 mM NaCl]. The concentration of RORγ-LBD was approximately 30 μM in PBS. Five-fold molar excess of NHS-LC-Biotin (Pierce) was added in a minimal volume of PBS. This solution was incubated with occasional gentle mixing for 60 minutes at ambient rt. The modified RORγ-LBD was dialyzed against 2 buffer changes—TBS pH 8.0 containing 5 mM DTT, 2 mM EDTA and 2% sucrose—each at least 20 times of the volume. The modified protein was distributed into aliquots, frozen on dry ice and stored at −80° C. The biotinylated RORγ-LBD was subjected to mass spectrometric analysis to reveal the extent of modification by the biotinylation reagent. In general, approximately 95% of the protein had at least a single site of biotinylation and the overall extent of biotinylation followed a normal distribution of multiple sites ranged from one to five.

A biotinylated peptide corresponding to amino acid 676 to 700 (CPSSHSSLTERHKILHRLLQEGSPS) (SEQ ID No: 2) of the co-activator steroid receptor coactivator SRC1(2) was generated using similar method.

### Assay

Preparation of Europium labeled SRC1(2) peptide: biotinylated SRC1(2) solution was prepared by adding an appropriate amount of biotinylated SRC1(2) from the 100 μM stock solution to a buffer containing 10 mM of freshly added DTT from solid to give a final concentration of 40 nM. An appropriate amount of Europium labeled Streptavidin was then added to the biotinylated SRC1(2) solution in a tube to give a final concentration of 10 nM. The tube was inverted gently and incubated for 15 minutes at rt. Twenty-fold excess biotin from the 10 mM stock solution was added and the tube was inverted gently and incubated for 10 minutes at rt.

Preparation of APC labeled RORγ-LBD: biotinylated RORγ-LBD solution was prepared by adding an appropriate amount of biotinylated RORγ-LBD from the stock solution to a buffer containing 10 mM of freshly added DTT from solid to give a final concentration of 40 nM. An appropriate amount of APC labeled Streptavidin was then added to the biotinylated RORγ-LBD solution in a tube to give a final concentration of 20 nM. The tube was inverted gently and incubated for 15 minutes at rt. Twenty-fold excess biotin from the 10 mM stock solution was then added and the tube was inverted gently and incubated for 10 minutes at rt.

Equal volumes of the above-described Europium labeled SRC1(2) peptide and the APC labeled RORγ-LBD were gently mixed together to give 20 nM RORγ-LBD, 10 nM APC-Strepavidin, 20 nM SRC1(2) and 5 nM Europium-Streptavidin. The reaction mixtures were incubated for 5 minutes. Using a Thermo Combi Multidrop 384 stacker unit, 25 μL of the reaction mixtures per well was added to the 384-well assay plates containing 1 μL of test compound per well in 100% DMSO. The plates were incubated for 1 hour and then read on ViewLux in Lance mode for EU/APC.

### RESULTS

All exemplified compounds (Examples 1-48) were tested in the dual FRET assay described above and were found to have a pIC₅₀ between 5 and 9.

1. A compound according to Formula (I): 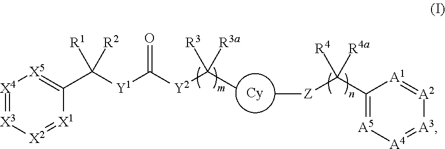
wherein:
m is 0, 1, or 2;
n is 0, 1, 2, or 3;
X¹, X², X³, X⁴, and X⁵ are each independently selected from N, N⁺—O⁻, CH, and CR⁵, wherein 0-3 of X¹, X², X³, X⁴, and X⁵ are N or N⁺—O⁻ and 1-3 of X¹, X², X³, X⁴, and X⁵ are CR⁵;
one of Y¹ and Y² is O or NR⁸ and the other is a bond;
or X¹ is CR⁵, Y¹ is NR⁸, Y² is a bond, and R⁵ and R⁸ taken together with the atoms to which they are attached form a five to seven membered ring, optionally containing an additional heteroatom selected from oxygen, nitrogen, and sulfur, which ring is optionally substituted by (C₁-C₄)alkyl;
Cy is (C₃-C₈)cycloalkyl, heterocycloalkyl, phenyl, or 5- or 6-membered heteroaryl, each of which is optionally substituted one, two, or three times, independently, by (C₁-C₆)alkyl, (C₁-C₆)haloalkyl, (C₃-C₆)cycloalkyl, halogen, oxo, cyano, hydroxyl, hydroxy(C₁-C₆)alkyl, (C₁-C₆)alkoxy, —((C₀-C₃)alkyl)NHCO₂R⁷, —((C₀-C₃)alkyl)N((C₁-C₄)alkyl)C₂R⁷, —((C₀-C₃)alkyl)NHC(O)R⁷, —((C₀-C₃)alkyl)N((C₁-C₄)alkyl)C(O)R⁷, —((C₀-C₃)alkyl)CO₂R⁷, —((C₀-C₃)alkyl)CONR⁷R⁸, —((C₀-C₃)alkyl)C(O)R⁷, (C₁-C₄)alkoxy(C₁-C₆)alkyl, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl(C₁-C₄)alkyl)amino, aryl, heteroaryl, aryl(C₁-C₆)alkyl, heteroaryl(C₁-C₆)alkyl, or heterocycloalkyl;
Z is O, S, SO₂, C═O, NR⁶, or a bond;
A¹, A², A³, A⁴, and A⁵ are each independently selected from N, N⁺—O⁻, CH, and CR¹⁰, wherein 0-3 of A¹, A², A³, A⁴, and A⁵ are N or N⁺—O⁻ and 0-3 of A¹, A², A³, A⁴, and A⁵ are CR¹⁰;
R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁵;
R² is hydrogen, (C₁-C₆)alkyl, or (C₁-C₆)haloalkyl;
or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁵;
R³ and R^{3a} are each independently hydrogen, hydroxyl, (C₁-C₆)alkyl, (C₁-C₆)haloalkyl, halogen, (C₁-C₆)alkoxy, amino, (C₁-C₄)alkylamino, or ((C₁-C₄)alkyl)((C₁-C₄)alkyl)amino;
each R⁴ is independently selected from hydrogen, halogen, (C₁-C₆)alkyl, (C₁-C₆)haloalkyl, —CO₂R⁷, —CONR⁷R⁸, —OR⁹, and —NR⁸R⁹, wherein said (C₁-C₆)alkyl or (C₁-C₆)haloalkyl is optionally substituted by hydroxyl, —OR⁹, —CO₂R⁷, —CONR⁷R⁸, or —NR⁸R⁹;
each R^{4a} is independently selected from hydrogen, halogen, hydroxyl, amino, and (C₁-C₆)alkyl;
or R⁴ and R^{4a} taken together with the carbon atom to which they are attached form a three to eight membered ring, optionally containing a heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂R⁷, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;
each R⁵ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;
R⁶ is hydrogen, (C₁-C₆)alkyl, (C₁-C₆)haloalkyl, (C₃-C₆)cycloalkyl, hydroxy(C₁-C₆)alkyl, (C₁-C₄)alkoxy(C₁-C₆)alkyl, —((C₀-C₃)alkyl)CO₂R⁷, —((C₀-C₃)alkyl)CONR⁷R⁸, aryl, heteroaryl, aryl(C₁-C₆)alkyl, heteroaryl(C₁-C₆)alkyl, or heterocycloalkyl;
R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;
R⁸ is hydrogen, (C₁-C₆)alkyl, or (C₁-C₆)haloalkyl;
or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;
R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by —CO₂R⁷, —CONH₂, —CONH(C₁-C₄)alkyl, —CON((C₁-C₄)alkyl)((C₁-C₄)alkyl), hydroxyl, (C₁-C₄)alkoxy, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;
or R⁸ and R⁹ taken together with the nitrogen atom to which they are attached form a four to eight membered ring, optionally containing an additional heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂H, —CO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷; and
R¹⁰ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, —((C₀-C₃)alkyl)CO₂R⁷, —((C₀-C₃)alkyl)CONR⁷R⁸, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;
or a salt thereof.

2. The compound or salt according to claim 1, wherein m is 1 and n is 1 or 2.

3. The compound or salt according to claim 1, wherein X¹ and X⁵ are each independently selected from N, N⁺—O⁻, CH, and CR⁵, and X², X³, and X⁴ are each independently selected from CH and CR⁵.

4. The compound or salt according to claim 1, wherein X¹ is a carbon atom substituted by halogen, (C₁-C₄)alkyl, (C₁-C₄)haloalkyl, cyano, (C₁-C₄)alkoxy, or ((C₁-C₄)alkyl)((C₁-C₄)alkyl)amino, and X², X³, X⁴, and X⁵ are each independently a carbon atom substituted by hydrogen, halogen, (C₁-C₄)alkyl, (C₁-C₄)haloalkyl, cyano, (C₁-C₄)alkoxy, or ((C₁-C₄)alkyl)((C₁-C₄)alkyl)amino, wherein 2-4 of X², X³, X⁴, and X⁵ are a carbon atom substituted by hydrogen.

5. The compound or salt according to claim 1, wherein Y¹ is NH or NCH₃ and Y² is a bond.

6. (canceled)

7. The compound or salt according to claim 1, wherein Cy is piperidinyl, piperazinyl, phenyl, pyridinyl, pyridazinyl, pyrazinyl, or pyrimidinyl, each of which is optionally substituted one or two times, independently, by (C₁-C₆)alkyl, (C₁-C₆)haloalkyl, halogen, cyano, (C₁-C₄)alkoxy, (C₁-C₄)alkyl)((C₁-C₄)alkyl)amino, —((C₀-C₃)alkyl)CO₂H, —((C₀-C₃)alkyl)CO₂(C₁-C₆)alkyl, or —((C₀-C₃)alkyl)CONH(C₁-C₆)alkyl.

8. The compound or salt according to claim 1, wherein Cy is phenyl, which is optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino.

9. The compound or salt according to claim 1, wherein Z is a bond, O, or NH.

10. The compound or salt according to claim 1, wherein R⁷ is (C₃-C₆)alkyl, (C₃-C₆)cycloalkyl, phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl, wherein said phenyl, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, or triazinyl is optionally substituted one or two times, independently, by halogen, (C₁-C₄)alkyl, (C₁-C₄)alkoxy, or ((C₁-C₄)alkyl)((C₁-C₄)alkyl)amino.

11. The compound or salt according to claim 1, wherein R¹ is phenyl or pyridinyl, each of which is optionally substituted one or two times, independently, by halogen, (C₁-C₄)alkyl, (C₁-C₄)alkoxy, or ((C₁-C₄)alkyl)((C₁-C₄)alkyl)amino.

12. The compound or salt according to claim 1, wherein R² is hydrogen or methyl.

13. The compound or salt according to claim 1, wherein R³ and R^{3a} are each independently hydrogen or methyl.

14. The compound or salt according to claim 1, wherein each R⁴ is independently selected from hydrogen, (C₁-C₄)alkyl, (C₁-C₄)haloalkyl, —OR⁹, and —NR⁸R⁹, wherein said (C₁-C₄)alkyl or (C₁-C₄)haloalkyl is optionally substituted by hydroxyl, —OR⁹, —CO₂R⁷, —CONR⁷R⁸, or —NR⁸R⁹.

15. The compound or salt according to claim 1, wherein each R⁴ is independently selected from hydrogen, (C₁-C₄)alkyl, (C₁-C₄)alkoxy, hydroxy(C₂-C₄)alkoxy, (C₁-C₄)alkoxy(C₂-C₄)alkoxy, amino(C₂-C₄)alkoxy, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₄)alkyl, and —O((C₁-C₃)alkyl)CON((C₁-C₄)alkyl)((C₁-C₄)alkyl).

16. The compound or salt according to claim 1, wherein each R^{4a} is independently selected from hydrogen and methyl.

17. (canceled)

18. The compound or salt according to claim 1, wherein A² and A⁴ are each independently selected from N, N⁺—O⁻, CH, and C((C₁-C₄)alkyl), and A¹, A³, and A⁵ are each independently selected from CH and C((C₁-C₄)alkyl), wherein at least one of A² and A⁴ is N⁺—O.

19. A compound according to Formula (Ia): 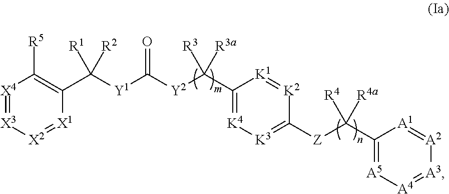
wherein:
m is 1;
n is 1 or 2;
X¹, X², X³, and X⁴ are each independently selected from N, N⁺—O⁻, CH, and CR⁵, wherein 0-2 of X¹, X², X³, and X⁴ are N or N⁺—O⁻ and 0-2 X¹, X², X³, and X⁴ are CR⁵;
Y¹ is NH or NCH₃ and Y² is a bond;
K¹, K², K³, and K⁴ are each independently selected from N, N⁺—O⁻, CH, and CR¹⁰, wherein 0-2 of K¹, K², K³, and K⁴ are N or N⁺—O⁻ and 0-2 of K¹, K², K³, and K⁴ are CR¹⁰;
Z is O, NR⁶, or a bond;
A¹, A², A³, A⁴, and A⁵ are each independently selected from N, N⁺—O⁻, CH, and CR¹⁰, wherein 0-3 of A¹, A², A³, A⁴, and A⁵ are N or N⁺—O⁻ and 0-3 of A¹, A², A³, A⁴, and A⁵ are CR¹⁰;
R¹ is (C₃-C₆)alkyl, (C₃-C₆)haloalkyl, (C₃-C₈)cycloalkyl, (C₃-C₆)alkoxy, (C₁-C₆)alkoxy(C₁-C₂)alkyl, aryl, heteroaryl, aryl(C₁-C₆)alkyl, heteroaryl(C₁-C₆)alkyl, or heterocycloalkyl, each of which is optionally substituted one, two, or three times, independently, by R⁵;
R² is hydrogen, (C₁-C₆)alkyl, or (C₁-C₆)haloalkyl;
or R¹ and R² taken together with the carbon atom to which they are attached form a three to eight membered ring, optionally containing a heteroatom selected from oxygen, nitrogen, and sulfur, which ring is optionally substituted one, two, or three times, independently, by R⁵;
R³ and R^{3a} are each independently hydrogen, hydroxyl, (C₁-C₄)alkyl, (C₁-C₄)haloalkyl, halogen, (C₁-C₄)alkoxy, amino, (C₁-C₄)alkylamino, or ((C₁-C₄)alkyl(C₁-C₄)alkyl)amino;
each R⁴ is independently selected from hydrogen, halogen, (C₁-C₄)alkyl, (C₁-C₄)haloalkyl, —OR⁹, and —NR⁸R⁹, wherein said (C₁-C₄)alkyl or (C₁-C₄)haloalkyl is optionally substituted by hydroxyl, —OR⁹, —CO₂R⁷, —CONR⁷R⁸, or —NR⁸R⁹;
each R^{4a} is independently selected from hydrogen, halogen, hydroxyl, amino, and (C₁-C₄)alkyl;
each R⁵ is independently selected from (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, and heterocycloalkyl;
R⁶ is hydrogen, (C₁-C₆)alkyl, (C₁-C₆)haloalkyl, (C₃-C₆)cycloalkyl, hydroxy(C₁-C₆)alkyl, (C₁-C₄)alkoxy(C₁-C₆)alkyl, —((C₀-C₃)alkyl)CO₂R⁷, —((C₀-C₃)alkyl)CONR⁷R⁸, aryl, heteroaryl, aryl(C₁-C₆)alkyl, heteroaryl(C₁-C₆)alkyl, or heterocycloalkyl;
R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl;
R⁸ is hydrogen, (C₁-C₆)alkyl, or (C₁-C₆)haloalkyl;
or R⁷ and R⁸ taken together with the nitrogen atom to which they are attached form a four to eight membered ring, optionally containing an additional heteroatom selected from oxygen, nitrogen, and sulfur, which ring is optionally substituted by (C₁-C₄)alkyl, (C₁-C₄)haloalkyl, (C₃-C₆)cycloalkyl, —CO₂H, —CO₂(C₁-C₄)alkyl, hydroxyl, hydroxy(C₁-C₆)alkyl, (C₁-C₄)alkoxy, (C₁-C₄)alkoxy(C₁-C₆)alkyl, amino, (C₁-C₄)alkylamino, or ((C₁-C₄)alkyl)((C₁-C₄)alkyl)amino;
R⁹ is —C(O)R⁷, —CO₂R⁷, —C(O)NR⁷R⁸, (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl, wherein said (C₁-C₆)alkyl, (C₁-C₆)haloalkyl, (C₃-C₆)cycloalkyl, aryl, heteroaryl, aryl(C₁-C₆)alkyl, heteroaryl(C₁-C₆)alkyl, or heterocycloalkyl is optionally substituted by —CO₂R⁷, —CONH₂, —CONH(C₁-C₄)alkyl, —CON((C₁-C₄)alkyl)((C₁-C₄)alkyl), hydroxyl, (C₁-C₄)alkoxy, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷;
or R⁸ and R⁹ taken together with the nitrogen atom to which they are attached form a four to eight membered ring, optionally containing an additional heteroatom selected from oxygen, nitrogen, and sulfur, which ring is optionally substituted by cyano, (C₁-C₄)alkyl, (C₁-C₄)haloalkyl, (C₃-C₆)cycloalkyl, —CO₂H, —CO₂(C₁-C₄)alkyl, —CONR⁷R⁸, hydroxyl, hydroxy(C₁-C₆)alkyl, (C₁-C₄)alkoxy, (C₁-C₄)alkoxy(C₁-C₆)alkyl, amino, (C₁-C₄)alkylamino, ((C₁-C₄)alkyl)((C₁-C₄)alkyl)amino, —NHCO₂R⁷, —N((C₁-C₄)alkyl)CO₂R⁷, —NHC(O)R⁷, or —N((C₁-C₄)alkyl)C(O)R⁷; and
R¹⁰ is (C₁-C₆)alkyl, (C₁-C₆)haloalkyl, (C₃-C₆)cycloalkyl, halogen, cyano, hydroxyl, hydroxy(C₁-C₆)alkyl, (C₁-C₆)alkoxy, (C₁-C₄)alkoxy(C₁-C₆)alkyl, —((C₀-C₃)alkyl)CO₂R⁷, —((C₀-C₃)alkyl)CONR⁷R⁸, amino(C₁-C₆)alkyl, ((C₁-C₄)alkyl)((C₁-C₄)alkyl)amino(C₁-C₆)alkyl, (C₁-C₄)alkylamino(C₁-C₆)alkyl, amino, (C₁-C₄)alkylamino, ((C₁-C₄)alkyl)((C₁-C₄)alkyl)amino, aryl, heteroaryl, aryl(C₁-C₆)alkyl, heteroaryl(C₁-C₆)alkyl, or heterocycloalkyl;
or a pharmaceutically acceptable salt thereof.

20. The compound or salt according to claim 19, wherein:
X¹, X², X³, and X⁴ are each independently a carbon atom substituted by hydrogen, halogen, cyano, (C₁-C₄)alkyl, (C₁-C₄)haloalkyl, (C₁-C₄)alkoxy, or ((C₁-C₄)alkyl)((C₁-C₄)alkyl)amino, wherein 2-4 of X¹, X², X³, and X⁴ are a carbon atom substituted by hydrogen;
K¹, K², K³, and K⁴ are each independently a carbon atom substituted by hydrogen, halogen, (C₁-C₄)alkyl, (C₁-C₄)alkoxy, or ((C₁-C₄)alkyl)((C₁-C₄)alkyl)amino, wherein 2-4 of K¹, K², K³, and K⁴ are a carbon atom substituted by hydrogen;
Z is O, NH, —N(C₁-C₄)alkyl, —N((C₀-C₃)alkyl)CO₂R⁷, —N((C₀-C₃)alkyl)CONR⁷R⁸, or a bond;
A² and A⁴ are each independently selected from N, N⁺—O⁻, CH, and C((C₁-C₄)alkyl), and A¹, A³, and A⁵ are each independently selected from CH and C((C₁-C₄)alkyl), wherein at least one of A² and A⁴ is N or N⁺—O⁻;
R² is hydrogen;
R³ and R^{3a} are each independently hydrogen or methyl;
each R⁴ is independently selected from hydrogen, (C₁-C₄)alkyl, (C₁-C₄)alkoxy, hydroxy(C₂-C₄)alkoxy, (C₁-C₄)alkylamino, ((C₁-C₄)alkyl)((C₁-C₄)alkyl)amino, (C₁-C₄)alkoxy(C₁-C₄)alkylamino, (C₁-C₄)alkoxy(C₂-C₄)alkoxy, amino(C₂-C₄)alkoxy, —O((C₁-C₃)alkyl)CO₂H, —O((C₁-C₃)alkyl)CO₂(C₁-C₄)alkyl, —O((C₁-C₃)alkyl)CONH₂, —O((C₁-C₃)alkyl)CONH(C₁-C₄)alkyl, and —O((C₁-C₃)alkyl)CON((C₁-C₄)alkyl)((C₁-C₄)alkyl);
each R^{4a} is independently selected from hydrogen, hydroxyl, amino, and (C₁-C₄)alkyl;
R⁷ is hydrogen, (C₁-C₆)alkyl, (C₁-C₆)haloalkyl, (C₃-C₆)cycloalkyl, (C₁-C₄)alkoxy(C₁-C₆)alkyl, aryl, heteroaryl, aryl(C₁-C₆)alkyl, heteroaryl(C₁-C₆)alkyl, or heterocycloalkyl; and
R⁸ is hydrogen, (C₁-C₆)alkyl, or (C₁-C₆)haloalkyl.

21. The compound or salt according to claim 20, wherein:
Z is O, NH, —N(C₁-C₄)alkyl, or a bond;
R¹ is phenyl optionally substituted one or two times, independently, by halogen, (C₁-C₄)alkyl, (C₁-C₄)haloalkyl, cyano, (C₁-C₄)alkoxy, or ((C₁-C₄)alkyl)((C₁-C₄)alkyl)amino; and
each R⁴ is independently selected from hydrogen, (C₁-C₄)alkyl, (C₁-C₄)alkylamino, ((C₁-C₄)alkyl(C₁-C₄)alkyl)amino, and (C₁-C₄)alkoxy.

22. (canceled)

23. (canceled)

24. A pharmaceutical composition comprising the compound, or pharmaceutically acceptable salt thereof, according to claim 19 and a pharmaceutically acceptable excipient.

25-35. (canceled)
